SEQUENCE LISTING (59 sequences)

SEQ ID NO: 1 - DNA, 21 bp, Artificial Sequence
  Synthetically generated oligonucleotide
cuuacgcugaguacuucgann

SEQ ID NO: 2 - DNA, 21 bp, Artificial Sequence
  Synthetically generated oligonucleotide
ucgaaguacucagcguaagnn

SEQ ID NO: 3 - DNA, 21 bp, Artificial Sequence
  Synthetically generated oligonucleotide
cuuacgcugaguacuucgann

SEQ ID NO: 4 - DNA, 21 bp, Artificial Sequence
  Synthetically generated oligonucleotide
ucgaaguacucagcguaagnn

SEQ ID NO: 5 - DNA, 21 bp, Artificial Sequence
  Synthetically generated oligonucleotide
cuuacgcnnnnnacuucgann

SEQ ID NO: 6 - DNA, 21 bp, Artificial Sequence
  Synthetically generated oligonucleotide
cuuacgcugaguacuucgann

SEQ ID NO: 7 - DNA, 21 bp, Artificial Sequence
  Synthetically generated oligonucleotide
ucgaaguacucagcguaagnn

SEQ ID NO: 8 - DNA, 21 bp, Artificial Sequence
  Synthetically generated oligonucleotide
cuuacgcugaguacuucgann

SEQ ID NO: 9 - DNA, 21 bp, Artificial Sequence
  Synthetically generated oligonucleotide
nuuacgcugaguacuucgann

SEQ ID NO: 10 - DNA, 21 bp, Artificial Sequence
  Synthetically generated oligonucleotide
ncgaaguacucagcguaagnn

SEQ ID NO: 11 - DNA, 21 bp, Artificial Sequence
  Synthetically generated oligonucleotide
ucgaaguacucagcguaagnn

SEQ ID NO: 12 - DNA, 21 bp, Artificial Sequence
  Synthetically generated oligonucleotide
nuuacgcugaguacuucgann

SEQ ID NO: 13 - DNA, 21 bp, Artificial Sequence
  Synthetically generated oligonucleotide
ncgaaguacucagcguaagnn

SEQ ID NO: 14 - DNA, 21 bp, Artificial Sequence
  Synthetically generated oligonucleotide
ncgaaguacucagcgnaagnn

SEQ ID NO: 15 - DNA, 21 bp, Artificial Sequence
  Synthetically generated oligonucleotide
ncgaaguacucagcgnaagnn

SEQ ID NO: 16 - DNA, 21 bp, Artificial Sequence
  Synthetically generated oligonucleotide
cunacgcugagnacuucgann

SEQ ID NO: 17 - DNA, 21 bp, Artificial Sequence
  Synthetically generated oligonucleotide
ucgaagnacucagcgnaagnn

SEQ ID NO: 18 - DNA, 21 bp, Artificial Sequence
  Synthetically generated oligonucleotide
cunacgcugagnacuucgann

SEQ ID NO: 19 - DNA, 21 bp, Artificial Sequence
  Synthetically generated oligonucleotide
ucgaagnacucagcgnaagnn

SEQ ID NO: 20 - DNA, 21 bp, Artificial Sequence
  Synthetically generated oligonucleotide
nunacgcugagnacuucgann

SEQ ID NO: 21 - DNA, 21 bp, Artificial Sequence
  Synthetically generated oligonucleotide
ncgaagnacucagcgnaagnn

SEQ ID NO: 22 - DNA, 21 bp, Artificial Sequence
  Synthetically generated oligonucleotide
nunacgcugagnacuucgann

SEQ ID NO: 23 - DNA, 21 bp, Artificial Sequence
  Synthetically generated oligonucleotide
ncgaagnacucagcgnaagnn

SEQ ID NO: 24 - protein, 16 aa, Artificial Sequence
  Exemplary Cell Permeation Peptide
RQIKIWFQNRRMKWKK

SEQ ID NO: 25 - protein, 14 aa, Artificial Sequence
  Exemplary Cell Permeation Peptide
GRKKRRQRRRPPQC

SEQ ID NO: 26 - protein, 27 aa, Artificial Sequence
  Exemplary Cell Permeation Peptide
GALFLGWLGAAGSTMGAWSQPKKKRKV

SEQ ID NO: 27 - protein, 18 aa, Artificial Sequence
  Exemplary Cell Permeation Peptide
LLIILRRRIRKQAHAHSK

SEQ ID NO: 28 - protein, 26 aa, Artificial Sequence
  Exemplary Cell Permeation Peptide
GWTLNSAGYLLKINLKALAALAKKIL

SEQ ID NO: 29 - protein, 18 aa, Artificial Sequence
  Amphiphilic model peptide
KLALKLALKALKAALKLA

SEQ ID NO: 30 - protein, 9 aa, Artificial Sequence
  Exemplary Cell Permeation Peptide
RRRRRRRRR

SEQ ID NO: 31 - protein, 10 aa, Artificial Sequence
  Exemplary Cell Permeation Peptide
KFFKFFKFFK

SEQ ID NO: 32 - protein, 37 aa, Artificial Sequence
  Exemplary Cell Permeation Peptides
LLGDFFRKSKEKIGKEFKRIVQRIKDFLRNLVPRTES

SEQ ID NO: 33 - protein, 31 aa, Artificial Sequence
  Exemplary Cell Permeation Peptides
SWLSKTAKKLENSAKKRISEGIAIAIQGGPR

SEQ ID NO: 34 - protein, 30 aa, Artificial Sequence
  Exemplary Cell Permeation Peptides
ACYCRIPACIAGERRYGTCIYQGRLWAFCC

SEQ ID NO: 35 - protein, 36 aa, Artificial Sequence
  Exemplary Cell Permeation Peptides
DHYNCVSSGGQCLYSACPIFTKIQGTCYRGKAKCCK

SEQ ID NO: 36 - protein, 12 aa, Artificial Sequence
  Exemplary Cell Permeation Peptides
RKCRIVVIRVCR

SEQ ID NO: 37 - protein, 42 aa, Artificial Sequence
  Exemplary Cell Permeation Peptides
RRRPRPPYLPRPRPPPFFPPRLPPRIPPGFPPRFPPRFPGKR

SEQ ID NO: 38 - protein, 13 aa, Artificial Sequence
  Exemplary Cell Permeation Peptides
ILPWKWPWWPWRR

SEQ ID NO: 39 - protein, 16 aa, Artificial Sequence
  Synthetically generated peptide
AAVALLPAVLLALLAP

SEQ ID NO: 40 - protein, 11 aa, Artificial Sequence
  Synthetically generated peptide
AALLPVLLAAP

SEQ ID NO: 41 - protein, 13 aa, Human immunodeficiency virus
GRKKRRQRRRPPQ

SEQ ID NO: 42 - protein, 16 aa, Drosophila Antennapedia
RQIKIWFQNRRMKWKK

SEQ ID NO: 43 - DNA, 21 bp, Artificial Sequence
  Syntheticaly generated oligonucletide
cuuacgcugnnnacuucgann

SEQ ID NO: 44 - DNA, 21 bp, Artificial Sequence
  Syntheticaly generated oligonucletide
cuuacgcugaguacnnnnann

SEQ ID NO: 45 - DNA, 21 bp, Artificial Sequence
  Syntheticaly generated oligonucletide
cuuacgcugaguacuucnnnn

SEQ ID NO: 46 - DNA, 21 bp, Artificial Sequence
  Syntheticaly generated oligonucletide
cuunnnnngaguacuucgann

SEQ ID NO: 47 - DNA, 21 bp, Artificial Sequence
  Syntheticaly generated oligonucletide
cuuacgcugaguacuunnnnn

SEQ ID NO: 48 - DNA, 21 bp, Artificial Sequence
  Syntheticaly generated oligonucletide
cnnncgcugaguacuucgann

SEQ ID NO: 49 - DNA, 21 bp, Artificial Sequence
  Syntheticaly generated oligonucletide
cuuacgcugagnnnnucgann

SEQ ID NO: 50 - DNA, 21 bp, Artificial Sequence
  Syntheticaly generated oligonucletide
cuuacgcugaguacuucgann

SEQ ID NO: 51 - DNA, 21 bp, Artificial Sequence
  Syntheticaly generated oligonucletide
nnuacgcugaguacutcgann

SEQ ID NO: 52 - DNA, 21 bp, Artificial Sequence
  Synthetically generated oligonucleotide
nnnncgcugaguacuucgann

SEQ ID NO: 53 - DNA, 21 bp, Artificial Sequence
  Synthetically generated oligonucleotide
nngaaguacucagcguaagnn

SEQ ID NO: 54 - DNA, 21 bp, Artificial Sequence
  Synthetically generated oligonucleotide
unnnaguacucagcguaagnn

SEQ ID NO: 55 - DNA, 21 bp, Artificial Sequence
  Synthetically generated oligonucleotide
ucgaaguacucagcgunnnnn

SEQ ID NO: 56 - DNA, 21 bp, Artificial Sequence
  Synthetically generated oligonucleotide
ucgaaguacucagcgunnnnn

SEQ ID NO: 57 - DNA, 21 bp, Artificial Sequence
  Synthetically generated oligonucleotide
nnnnaguacucagcguaagnn

SEQ ID NO: 58 - DNA, 21 bp, Artificial Sequence
  Synthetically generated oligonucleotide
ucnnnguacucagcnnaagnn

SEQ ID NO: 59 - DNA, 21 bp, Artificial Sequence
  Synthetically generated oligonucleotide
ucgaaguacucagcgnnagnn

(19) United States
(12) Patent Application Publication
(10) Pub. No.: US 2005/0233342 A1
(43) Pub. Date: Oct. 20, 2005

(54) Methods of preventing off-target gene silencing

(76) Inventors: Muthiah Manoharan, Weston, MA (US); Kallanthottathil G. Rajeev, Cambridge, MA (US)

Correspondence Address: FISH & RICHARDSON PC, P.O. BOX 1022, MINNEAPOLIS, MN, 55440-1022, US

(21) Appl. No.: 10/899,912

(22) Filed: Jul. 26, 2004

Related U.S. Application Data
(63) Continuation-in-part of application No. PCT/US04/11225, filed on Apr. 9, 2004.
(63) Continuation-in-part of application No. PCT/US04/07070, filed on Mar. 8, 2004.
(63) Continuation-in-part of application No. PCT/US04/10586, filed on Apr. 5, 2004.
(60) Provisional application No. 60/462,097, filed on Apr. 9, 2003.
(60) Provisional application No. 60/461,915, filed on Apr. 10, 2003.
(60) Provisional application No. 60/463,772, filed on Apr. 17, 2003.
(60) Provisional application No. 60/465,802, filed on Apr. 25, 2003.
(60) Provisional application No. 60/493,986, filed on Aug. 8, 2003.
(60) Provisional application No. 60/494,597, filed on Aug. 11, 2003.
(60) Provisional application No. 60/506,341, filed on Sep. 26, 2003.
(60) Provisional application No. 60/518,453, filed on Nov. 7, 2003.
(60) Provisional application No. 60/469,612, filed on May 9, 2003.
(60) Provisional application No. 60/510,246, filed on Oct. 9, 2003.
(60) Provisional application No. 60/510,318, filed on Oct. 10, 2003.
(60) Provisional application No. 60/465,665, filed on Apr. 25, 2003.
(60) Provisional application No. 60/462,894, filed on Apr. 14, 2003.
(60) Provisional application No. 60/452,682, filed on Mar. 7, 2003.
(60) Provisional application No. 60/454,265, filed on Mar. 12, 2003.
(60) Provisional application No. 60/454,962, filed on Mar. 13, 2003.
(60) Provisional application No. 60/469,612, filed on May 9, 2003.
(60) Provisional application No. 60/463,772, filed on Apr. 17, 2003.
(60) Provisional application No. 60/462,894, filed on Apr. 14, 2003.
(60) Provisional application No. 60/455,050, filed on Mar. 13, 2003.
(60) Provisional application No. 60/465,802, filed on Apr. 25, 2003.
(60) Provisional application No. 60/465,665, filed on Apr. 25, 2003.
(60) Provisional application No. 60/493,986, filed on Aug. 8, 2003.
(60) Provisional application No. 60/510,246, filed on Oct. 9, 2003.
(60) Provisional application No. 60/510,318, filed on Oct. 10, 2003.
(60) Provisional application No. 60/494,597, filed on Aug. 11, 2003.
(60) Provisional application No. 60/506,341, filed on Sep. 26, 2003.
(60) Provisional application No. 60/518,453, filed on Nov. 7, 2003.
(60) Provisional application No. 60/460,783, filed on Apr. 3, 2003.
(60) Provisional application No. 60/463,772, filed on Apr. 17, 2003.
(60) Provisional application No. 60/465,802, filed on Apr. 25, 2003.
(60) Provisional application No. 60/469,612, filed on May 9, 2003.
(60) Provisional application No. 60/493,986, filed on Aug. 8, 2003.
(60) Provisional application No. 60/462,894, filed on Apr. 14, 2003.
(60) Provisional application No. 60/465,665, filed on Apr. 25, 2003.
(60) Provisional application No. 60/503,414, filed on Sep. 15, 2003.
(60) Provisional application No. 60/506,341, filed on Sep. 26, 2003.
(60) Provisional application No. 60/510,246, filed on Oct. 9, 2003.
(60) Provisional application No. 60/510,318, filed on Oct. 10, 2003.
(60) Provisional application No. 60/518,453, filed on Nov. 7, 2003.
(60) Provisional application No. 60/494,597, filed on Aug. 11, 2003.

(51) Int. Cl. C12Q 1/68; C12N 15/82

(52) U.S. Cl. 435/6; 435/468

(57) ABSTRACT

Aspects featured in the invention relate to compositions and methods for inhibiting off-target gene silencing by iRNA agents.

### RELATED APPLICATIONS

This application is a continuation in part of International Application No.: PCT/US04/011225, filed Apr. 9, 2004, which claims the benefit of U.S. Provisional Application No. 60/462,097, filed Apr. 9, 2003; U.S. Provisional Application No. 60/461,915, filed Apr. 10, 2003; U.S. Provisional Application No. 60/463,772, filed Apr. 17, 2003; U.S. Provisional Application No. 60/465,802, filed Apr. 25, 2003; U.S. Provisional Application No. 60/493,986, filed Aug. 8, 2003; U.S. Provisional Application No. 60/494,597, filed Aug. 11, 2003; U.S. Provisional Application No. 60/506,341, filed Sep. 26, 2003; U.S. Provisional Application No. 60/518,453, filed Nov. 7, 2003; U.S. Provisional Application No. 60/469,612, filed May 9, 2003; U.S. Provisional Application No. 60/510,246, filed Oct. 9, 2003; U.S. Provisional Application No. 60/510,318, filed Oct. 10, 2003; U.S. Provisional Application No. 60/465,665, filed Apr. 25, 2003; U.S. Provisional Application No. 60/462,894, filed Apr. 14, 2003; International Application No. PCT/US04/07070, filed Mar. 8, 2004; and International Application No. PCT/US04/10586, filed Apr. 5, 2004. International Application No. PCT/US04/07070 claims the benefit of U.S. Provisional Application No. 60/452,682, filed Mar. 7, 2003; U.S. Provisional Application No. 60/462,894, filed Apr. 14, 2003; U.S. Provisional Application No. 60/465,665, filed Apr. 25, 2003; U.S. Provisional Application No. 60/463,772, filed Apr. 17, 2003; U.S. Provisional Application No. 60/465,802, filed Apr. 25, 2003; U.S. Provisional Application No. 60/493,986, filed Aug. 8, 2003; U.S. Provisional Application No. 60/494,597, filed Aug. 11, 2003; U.S. Provisional Application No. 60/506,341, filed Sep. 26, 2003; U.S. Provisional Application No. 60/518,453, filed Nov. 7, 2003; U.S. Provisional Application No. 60/454,265, filed Mar. 12, 2003; U.S. Provisional Application No. 60/454,962, filed Mar. 13, 2003; U.S. Provisional Application No. 60/455,050, filed Mar. 13, 2003; U.S. Provisional Application No. 60/469,612, filed May 9, 2003; U.S. Provisional Application No. 60/510,246, filed Oct. 9, 2003; and U.S. Provisional Application No. 60/510,318, filed Oct. 10, 2003. International Application No. PCT/US04/10586 claims the benefit of U.S. Provisional Application No. 60/460,783, filed Apr. 3, 2003; U.S. Provisional Application No. 60/503,414, filed Sep. 15, 2003; U.S. Provisional Application No. 60/462,894, filed Apr. 14, 2003; U.S. Provisional Application No. 60/465,665, filed Apr. 25, 2003; U.S. Provisional Application No. 60/463,772, filed Apr. 17, 2003; U.S. Provisional Application No. 60/465,802, filed Apr. 25, 2003; U.S. Provisional Application No. 60/493,986, filed Aug. 8, 2003; U.S. Provisional Application No. 60/494,597, filed Aug. 11, 2003; U.S. Provisional Application No. 60/506,341, filed Sep. 26, 2003; U.S. Provisional Application No. 60/518,453, filed Nov. 7, 2003; U.S. Provisional Application No. 60/469,612, filed May 9, 2003; U.S. Provisional Application No. 60/510,246, filed Oct. 9, 2003; U.S. Provisional Application No. 60/510,318, filed Oct. 10, 2003; and International Application No. PCT/US04/07070, filed Mar. 8, 2004. The contents of all of these prior applications are hereby incorporated by reference in their entireties.

### TECHNICAL FIELD

This invention relates to methods and compositions for preventing off-target gene silencing by iRNA agents. More particularly, the invention relates to modification of the sense strand of an iRNA agent for the inhibition of off-target gene silencing.

### BACKGROUND

RNA interference or “RNAi” is a term initially coined by Fire and co-workers to describe the observation that double-stranded RNA (dsRNA) can block gene expression when it is introduced into worms (Fire et al., Nature 391:806-811, 1998). Short dsRNA directs gene-specific, post-transcriptional silencing in many organisms, including vertebrates, and has provided a new tool for studying gene function.

### SUMMARY

The invention features methods and compositions for silencing genes with minimal off-target gene silencing. Off-target silencing can be mediated by the sense strand, such as by RNAi, or other, e.g., an antisense mechanism. To minimize the effect of an iRNA agent on off-target silencing, the sense strand of the iRNA agent can be modified, such as at the 5′ or 3′ ends or at an internal site in the sense strand. Modifications at one, two, or all three of these sense strand locations can be useful for inhibiting off-target silencing.

One aspect of the invention features a method of preventing off-target gene silencing in a cell, which includes contacting a duplex RNA with the cell. The duplex RNA includes a modification on the sense strand, and (a) the sense strand of duplex RNA has a region of at least 70% complementarity for at least 10 nucleotides of a preselected gene; or (b) the modified or unmodified sense strand has been tested for an ability to silence the off-target gene. In one embodiment, the off-target gene is expressed in said cell, and in another embodiment, the off-target gene is expressed in a different cell type. The off-target gene can be, e.g., a housekeeping gene. The off-target gene can be a gene involved in respiration or cell-cycle regulation. The off-target gene can be a gene for which silencing or down-regulation is undesirable.

In another embodiment, the 5′ terminus of the sense strand of a duplex iRNA agent includes one or more chemical modifications. In one embodiment one or more L-nucleosides are present on the 5′ end, in which the nucleoside has a constituent L-sugar instead of a D-sugar (i.e., the sugar is related configurationally to L-glyceraldehyde instead of L-glyceraldehyde). In another aspect, one or more alpha-nucleosides are present on the 5′ end. In another embodiment one or more nucleotides at the 5′ terminus are joined by 2′-5′-linkages, instead of 3′-5′ linkages.

In another embodiment a conjugate, e.g., a conjugate described herein, is present on the 5′ terminus of the sense strand. The conjugate can be attached to the 5′ hydroxyl, and, preferably, a phosphate group (PO₄) does not link the conjugate and the sugar, unless, for example, it is modified to be more resistant to release of the conjugate. In certain embodiments, one or more modifications can be introduced that render the modified phosphate group more resistant to enzymatic degradation, e.g., by nucleases, relative to an unmodified phosphate group. For example, one or both nonlinking oxygen atoms of the phosphate group can be replaced by another atom or group of atoms, e.g., S, Se, BH₃⁻, H, alkoxy, aryloxy, a mono- or di-substituted amino group, alkyl, cycloalkyl, aralkyl, aryl, or heteroaryl group. Preferably, the modified phophate group is a phosphorothioate group. In certain embodiments, the conjugate can be linked to the sugar by a phosphonate moiety instead of a phosphate group, in which one or both of the linking oxygen atoms of the phosphate group can either be absent or replaced with, e.g. a substituted or unsubstituted alkylene, alkenylene, or alkynylene group having 1-20 (e.g., 1, 2, 3, 4, 5, 6, 7, 8, 9, 10, 11, 12, 13, 14, 15, 16, 17, 18, 19, or 20) carbon atoms in the case of alkylene and 2-20 (e.g., 2, 3, 4, 5, 6, 7, 8, 9, 10, 11, 12, 13, 14, 15, 16, 17, 18, 19, or 20) carbon atoms in the case of alkenylene or alkynylene. When the linking oxygen atom between the phosphate group and the sugar is absent, the sugar C-5 methylene group can be substituted with 1 or 2 halo (preferably, fluoro). In certain embodiments, the phosphate group can be replaced by a substituted or unsubstituted alkylene, alkenylene, or alkynylene group having 1-20 (e.g., 1, 2, 3, 4, 5, 6, 7, 8, 9, 10, 11, 12, 13, 14, 15, 16, 17, 18, 19, or 20) carbon atoms in the case of alkylene and 2-20 (e.g., 2, 3, 4, 5, 6, 7, 8, 9, 10, 11, 12, 13, 14, 15, 16, 17, 18, 19, or 20) carbon atoms in the case of alkenylene or alkynylene and at least 1 (e.g., at least 2, at least 3, at least 4, at least 5, at least 6, at least 6) heteroatom selected from nitrogen, oxygen, or sulfur. Heteroatoms can be at terminal and/or internal positions of carbon chain. Heteroatoms or heteroatom containing groups can be introduced, e.g., by displacement of a leaving group (mesylate, tosylate, triflate, halide) on a carbon chain with nitrogen, sulfur, or oxygen containing nucleophiles (e.g., NH₃, H₂S, H₂O, amine, thiol or alcohol (or synthetic equivalents or conjugate bases thereof)). Preferably, the phosphate group is replaced with NH₂(CH₂)ₓ—SH(CH₂)ₓ—, or OH(CH₂)ₓ—, in which x is 1, 2, or 3; or PEG

In another embodiment, the sense strand 5′ hydroxyl of an iRNA duplex includes a phosphonate linkage, wherein the 5′-OH-sugar is replaced by 5′-(PO₄)—X-sugar. “X” can be CH₂, CF₂, or CFH.

In another embodiment, one or more nucleotide bases are modified at the 5′ terminus. For example, a nucleotide base can be an N2-purine, N7-purine, or C5-pyrimidine.

In another embodiment, the terminal 5′ nucleotides are joined by 3′-5′ linkages, and one or more 2′ hydroxyls are replaced by OR, SR, NR2 or F. In one embodiment, the terminal nucleotides are joined by 2′-5′ linkages, and one or more 3′ hydroxyls are replaced by OR, SR, NR2 or F.

In another embodiment, the 5′ hydroxyl are replaced by ((SO₄)—CₙHₙ—O—) or (R₂N—CₙHₙ—O—).

In one aspect, the 3′ terminus of the sense strand of a duplex iRNA agent includes one or more chemical modifications. In one embodiment, a steroidal molecule, e.g., cholesterol, is attached to the 3′ terminus of the sense strand. The steroidal molecule can be attached by a cyclic or acyclic linker, e.g., a cationic linker that includes 3-12 (e.g., 2, 3, 4, 5, 6, 7, 8, 9, 10, 11, 12, 13, 14, 15, 16, 17, 18, 19, or 20) carbon atoms and nitrogen and oxygen containing functional groups (e.g., a primary hydroxyl group, a secondary hydroxyl group, and a primary amino group or secondary amino group), which can serve as direct or indirect (e.g., via a tether) attachment points for the steroidal molecule and sense strand. In certain embodiments, the linker can be, e.g., a pyrrolidine, pyrroline, piperidine, piperazine, decalin, indane, or a serinol-based linker. A steroidal molecule is a fat-soluble organic compound having as a basis 17 carbon atoms in four fused ring sytem. In one embodiment, the iRNA agent facilitates entry of the iRNA agent into a cell, e.g., by binding to a particular cell receptor, or by facilitating movement of the iRNA agent through the cell membrane. In another embodiment, the modification targets an iRNA agent to a particular tissue. For example, a cholesterol moiety conjugated to the 3′ terminus of the sense strand of an iRNA agent can direct the iRNA agent to the liver.

In one embodiment, the iRNA agent is at least 21 nucleotides long and includes a sense RNA strand and an antisense RNA strand, wherein the antisense RNA strand is 25 or fewer nucleotides in length, and the duplex region of the iRNA agent is 18-25 nucleotides in length. The iRNA agent may further include a nucleotide overhang having 1 to 4 unpaired nucleotides, and the unpaired nucleotides may have at least one phosphorothioate dinucleotide linkage. The nucleotide overhang can be, e.g., at the 3′ end of the antisense strand of the iRNA agent.

In one aspect, the sense strand of a duplex iRNA agent includes a modification in an internal region of the sequence. A nucleotide in an internal region is any nucleotide that is not on the 3′ or 5′ terminus of the sense strand. In one embodiment, the modification is a DNA modification, e.g., a deoxynucleotide replaces a ribonucleotide. For example, a deoxythymidine can replace uridine. In another embodiment, a ribonucleotide is modified. For example, a uridine can be replaced with 2′-arabino-fluorodeoxyuridine, or methylated 2′-arabino-fluorodeoxyuridine. Preferably, the internal nucloetide modification is one or more nucleotides away from the terminal nucleotide. More preferably, the nucleotide modification is two or more nucleotides away from the terminal nucleotide. Even more preferably, the nucleotide modification is three or more nucleotides away from the terminal nucleotide. In one embodiment, the nucleotide modification occurs in the same sense strand as one or more a phosphorothioate linkage.

In one embodiment, the iRNA agent is at least 21 nucleotides long and includes a sense RNA strand and an antisense RNA strand, wherein the antisense RNA strand is 25 or fewer nucleotides in length, and the duplex region of the iRNA agent is 18-25 nucleotides in length. The iRNA agent may further include a nucleotide overhang having 1 to 4 unpaired nucleotides, and the unpaired nucleotides may have at least one phosphorothioate dinucleotide linkage. The nucleotide overhang can be, e.g., at the 3′ end of the antisense strand of the iRNA agent.

In one aspect, the invention features a method of evaluating an agent, e.g., an agent of a type described herein, such as a double stranded iRNA agent that includes a sense strand modification.

In a preferred embodiment the method includes evaluating the agent in a first test system; and, if the agent demonstrates a desirable inhibition of target gene expresson and a low level of off-target silencing, evaluating the candidate in a second, preferably different, test system. In a particularly preferred embodiment the second test system includes administering the candidate agent to an animal and evaluating the effect of the candidate agent on target and off-target expression in the animal.

A test system can include: contacting the candidate agent with a target molecule, e.g., a target RNA or DNA, preferably in vitro, and determining if there is an interaction, e.g., binding of the candidate agent to the target, or modifying the target, e.g., by making or breaking a covalent bond in the target. Modification is correlated with the ability to modulate target gene expression while maintaining a low level of off-target gene expression. The test system can include contacting the candidate agent with a cell and evaluating modulation of target gene expression.

In one embodiment, target and off-target gene expression can be evaluated by a method to examine RNA levels (e.g., Northern blot analysis, RT-PCR, or RNAse protection assay) or protein levels (e.g., Western blot).

In one embodiment, e.g., as a second test, the agent is administered to an animal, e.g., a mammal, such as a mouse, rat, rabbit, human, or non-human primate, and the animal is monitored for an effect of the agent. For example, a tissue of the animal, e.g., a brain tissue or ocular tissue, is examined for an effect of the agent on target expression. The tissue can be examined for the presence of target RNA and/or protein, for example. In one embodiment, the animal is observed to monitor an improvement or stabilization of a symptom while having minimal unwanted side effects, e.g., toxicity, irritation or allergic response, which may be caused or exacerbated by off-target gene silencing. The agent can be administered to the animal by any method, e.g., orally, or by intrathecal or parenchymal injection, such as by stereoscopic injection into the brain.

In one embodiment, the invention features a method of evaluating a modification for an ability to inhibit off-target silencing by an iRNA agent, e.g., an iRNA agent described herein. The modification can be applied to the 5′, the 3′ end, or an internal nucleotide of the antisense strand of an iRNA agent duplex. The iRNA agent is then evaluated for its effect on target gene silencing. An antisense strand modification that decreases the silencing effect of an iRNA agent, can be applied to the sense strand of an iRNA agent to inhibit off-target silencing.

In one embodiment, the invention features a method of evaluating an iRNA agent, e.g., an iRNA agent described herein, such as an iRNA agent carrying a modification on the sense strand of an iRNA duplex. The method includes providing an iRNA agent; contacting the iRNA agent with a cell containing, and capable of expressing, a target gene; and evaluating the effect of the iRNA agent on target gene expression, e.g., by comparing target gene expression with a control, such as a control RNA in the cell. The method also includes monitoring off-target gene expression, e.g., by genomic (e.g., microarray) analysis to examine global RNA levels after administration of a candidate unmodified versus sense strand modified iRNA agent to identify off-target RNAs that are silenced by the unmodified agent, but not by the modified agent. In one embodiment, RNAs having sequence complementarity (e.g., 40%, 50%, 60%, 70%, 80%, 90%, or higher complementarity) to the sense strand of the iRNA agent are predicted to be subject to off-target silencing, and these RNA species can be monitored (e.g., by Northern blot, RT-PCR, or RNAse protection) for differences in expression levels following administration of an unmodified versus sense strand modified iRNA agent.

In another aspect, the invention features a method of evaluating a modification of a sense strand of a duplex RNA for the ability to inhibit silencing of an off-target gene. The method includes: (a) modifying the sense strand of the duplex RNA, in which the sense strand of the duplex RNA has a region of at least 70% complementarity to at least 10 nucleotides of the off-target gene; (b) contacting the modified sense strand to a cell expressing the off-target gene; and (c) comparing expression of the off-target gene to expression of the off-target gene following contact with an unmodified sense strand of the duplex RNA.

A “substantially identical” sequence includes a region of sufficient homology to a target gene, and is of sufficient length in terms of nucleotides, that the iRNA agent, or a fragment thereof, can mediate down regulation of the target gene. Thus, the iRNA agent, e.g., the antisense strand of an iRNA agent is or includes a region which is at least partially, and in some embodiments fully, complementary to a target RNA transcript. Likewise, an iRNA agent can include a region, e.g. a region on the sense strand, which is at least partially, and in some embodiments fully, complementary to an off-target RNA transcript. It is not necessary that there be perfect complementarity between the iRNA agent and the target (or off-target), but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more but preferably 6, 5, 4, 3, 2, or fewer mismatches (with respect to the target RNA). The mismatches, particularly in the antisense strand, are most tolerated in the terminal regions and if present are preferably in a terminal region or regions, e.g., within 6, 5, 4, or 3 nucleotides of the 5′ and/or 3′ terminus. The sense strand need only be sufficiently complementary with the antisense strand to maintain the overall double strand character of the molecule.

An “RNA agent” as used herein, is an unmodified RNA, modified RNA, or nucleoside surrogate, all of which are described herein. While numerous modified RNAs and nucleoside surrogates are described, preferred examples include those which have greater resistance to nuclease degradation than do unmodified RNAs. Preferred examples include those that have a 2′ sugar modification, a modification in a single strand overhang, preferably a 3′ single strand overhang, or, particularly if single stranded, a 5′ modification which includes one or more phosphate groups or one or more analogs of a phosphate group. Preferably a duplex region of an RNA agent includes a modification, e.g., a modification described herein, on the sense strand.

An “iRNA agent” (“interfering RNA agent”) as used herein, is an RNA agent, which can downregulate the expression of a target gene, preferably an endogenous or pathogen target RNA. While not wishing to be bound by theory, an iRNA agent may act by one or more of a number of mechanisms, including post-transcriptional cleavage of a target mRNA sometimes referred to in the art as RNAi, or pre-transcriptional or pre-translational mechanisms. An iRNA agent can include a single strand or can include more than one strands, e.g., it can be a double stranded iRNA agent. If the iRNA agent is a single strand it is particularly preferred that it include a 5′ modification which includes one or more phosphate groups or one or more analogs of a phosphate group. Preferably a duplex region of an RNA agent includes a modification, e.g., a modification described herein, on the sense strand of a duplex. An iRNA agent is also referred to herein as a short interfering RNA (siRNA) or a dsRNA.

The details of one or more embodiments of the invention are set forth in the accompanying drawings and the description below. Other features, objects, and advantages of the invention will be apparent from this description, and from the claims. This application incorporates all cited references, patents, and patent applications by references in their entirety for all purposes.

### BRIEF DESCRIPTION OF DRAWINGS

FIG. 1 is a schematic illustrating the conjugation of a cholesterol moiety to the 3′ end of a dsRNA via pyrrolidine linker. The sphere represents a solid support synthesis reagent.

FIG. 2 is a graph depicting the effect of unmodified (diamonds) versus modified (squares) dsRNAs on firefly luciferase gene expression. The modified siRNA contains a cholesterol moiety on the 3′ end of the sense strand.

FIG. 3 is a graph depicting the effect of unmodified versus modified dsRNAs on firefly luciferase gene expression. 1S-1S dsRNA (gray circles) is unmodified; 11S-11AS dsRNA (triangles) carries a cholesterol moiety at the 3′ end of both the sense and antisense dsRNA strands; 1S-11AS dsRNA (squares) has an unmodified sense strand, and the antisense strand carries a cholesterol moiety on the 3′ end; and 11S-1AS dsRNA (diamonds) has an unmodified antisense strand, and the sense strand carries a cholesterol moiety on the 3′ end. The sequences of 11S and 11AS dsRNA strands are shown. The sequences are the same for 1S and 1AS strands, but these latter sequences do not include the cholesterol moiety.

FIG. 4 is a graph depicting the effect of cholesterol on firefly luciferase gene silencing when the cholesterol is conjugated to the 5′ terminus of the sense strand.

FIG. 5 is a graph depicting the effect of the linker used to conjugate cholesterol to the dsRNAs of FIG. 3. GL3 dsRNA (X's) is unmodified; *S-*AS dsRNAs (triangles) carry the linker on both the sense and antisense strands; *S-AS dsRNAs (squares) only carry the linker on the sense strand; S-*AS dsRNAs (diamonds) only carry the linker on the antisense strand.

FIG. 6 is a schematic illustrating the conjugation of a naproxen moiety to the 3′ end of a dsRNA.

FIG. 7 is a graph depicting the effect of 3′ sense strand-conjugated naproxen (“Nap”). 1S-1AS dsRNAs (X's) are unmodified; 13S-13AS dsRNAs (triangles) carry naproxen on both the sense and antisense strands; 1S-13AS (squares) only carries naproxen on the antisense strand; 13S-1AS (diamonds) only carries naproxen on the sense strand.

FIG. 8 is a graph comparing the effect of 3′ sense strand-conjugated naproxen (“Nap”) and cholesterol on cellular uptake.

FIG. 9 is a graph depicting the effect of a specific linker used to conjugate cholesterol to the 3′ antisense strand of dsRNAs. GL3 dsRNAs (X's) are unmodified; Chol-(pyrr) dsRNAs (triangles) carry cholesterol attached by a pyrrolidine linker; Ibu-(ser) dsRNAs (squares) carry ibuprofen attached by a serinol linker; Chol-(ser) dsRNAs (diamonds) carry cholesterol attached by a pyrrolidine linker.

FIG. 10 is a graph depicting the effect of an L-sugar placed at the 5′ terminus of the sense versus the antisense strand of a dsRNA. 1000/2077 dsRNAs (black X's) have an L-sugar on the 5′ terminus of the antisense strand; 1000/1001 dsRNAs (gray X's) are unmodified; 2076/1001 dsRNAs (squares) have an L-sugar on the 5′ terminus of the sense strand.

FIG. 11 is a graph depicting the effect of a 2′-5′ linkage (“#*” at the 5′ terminus of the sense versus the antisense strand of a dsRNA. 1000/1001 dsRNAs (gray X's) are unmodified; 1000/2075 dsRNAs (triangles) have a 2′-5′ linkage on the 5′ terminus of the antisense strand; 2074/1001 dsRNAs (diamonds) have a 2′-5′ linkage on the 5′ terminus of the sense strand.

FIG. 12 is a graph depicting the effect of a DNA modification in the internal region of the sense versus the antisense strand of a dsRNA. 1000/1001 dsRNAs (triangles) are unmodified; 1000/2365 dsRNAs (squares) have a phosphorothioate linkage (“*”) in the internal region of the antisense strand; and 1000/2366 dsRNAs (diamonds) have a DNA modification (“dT”) in the internal region of the antisense strand.

FIG. 13 is a graph depicting the effect of modifications in the internal region of the sense versus the antisense strand of a dsRNA. 1000/1001 dsRNAs (X's) are unmodified; two uridines in the sense and antisense sequences of 2484/2485 dsRNAs (triangles) have been replaced by 2′-arabino-fluorodeoxyuridine (aUf) nucleotides; two uridines in the sense sequence of 2484/1001 dsRNAs (squares) have been replaced by 2′-arabino-fluorodeoxyuridine nucleotides; and two uridines in the antisense sequences of 1000/2485 dsRNAs (diamonds) have been replaced by 2′-arabino-fluorodeoxyuridine nucleotides.

FIG. 14 is a graph depicting the effect of modifications in the internal region of the sense versus the antisense strand of a dsRNA. 1000/1001 dsRNAs (black X's) are unmodified; two uridines in the sense and antisense sequences of 2484PS/2485PS dsRNAs (gray X's) have been replaced by 2′-arabino-fluorodeoxyuridine (aUf) nucleotides, and phosphorothioate linkages (*) are incorporated into the 5′ and 3′ terminal regions; two uridines in the sense and antisense sequences of 2482PS/2483PS dsRNAs (triangles) have been replaced by methylated 2′-arabino-fluorodeoxyuridine (^{5Me}aUF) nucleotides and phosphorothioate linkages are incorporated into the 5′ and 3′ terminal regions; two uridines in the sense and antisense sequences of 2484/2485 dsRNAs (squares) have been replaced by 2′-arabino-fluorodeoxyuridine (aUf) nucleotides; two uridines in the sense and antisense sequences of 2482/2483 dsRNAs (diamonds) have been replaced by methylated 2′-arabino-fluorodeoxyuridine nucleotides.

### DETAILED DESCRIPTION

Double-stranded (dsRNA) directs the sequence-specific silencing of mRNA through a process known as RNA interference (RNAi). The process occurs in a wide variety of organisms, including mammals and other vertebrates.

It has been demonstrated that 21-23 nt fragments of dsRNA are sequence-specific mediators of RNA silencing, e.g., by causing RNA degradation. While not wishing to be bound by theory, it may be that a molecular signal, which may be merely the specific length of the fragments, present in these 21-23 nt fragments, recruits cellular factors that mediate RNAi. Described herein are methods for preparing and administering these 21-23 nt fragments, and other iRNA agents, and their use for specifically inactivating gene function, and the function of the SNCA gene in particular. The use of iRNA agents (or recombinantly produced or chemically synthesized oligonucleotides of the same or similar nature) enables the targeting of specific mRNAs for silencing in mammalian cells. In addition, longer dsRNA agent fragments can also be used, e.g., as described below.

Although, in mammalian cells, long dsRNAs can induce the interferon response which is frequently deleterious, short dsRNAs (sRNAs) do not trigger the interferon response, at least not to an extent that is deleterious to the cell and host. In particular, the length of the iRNA agent strands in an sRNA agent can be less than 31, 30, 28, 25, or 23 nt, e.g., sufficiently short to avoid inducing a deleterious interferon response. Thus, the administration of a composition of sRNA agent (e.g., formulated as described herein) to a mammalian cell can be used to silence expression of a target gene while circumventing the interferon response. Further, use of a discrete species of iRNA agent can be used to selectively target one allele of a target gene, e.g., in a subject heterozygous for the allele.

Moreover, in one embodiment, a mammalian cell is treated with an iRNA agent that disrupts a component of the interferon response, e.g., dsRNA-activated protein kinase PKR. Such a cell can be treated with a second iRNA agent that includes a sequence complementary to a target RNA and that has a length that might otherwise trigger the interferon response.

In a typical embodiment, the subject is a mammal such as a cow, horse, mouse, rat, dog, pig, goat, or a primate. In a much preferred embodiment, the subject is a human, e.g., a normal individual or an individual that has, is diagnosed with, or is predicted to have a disease or disorder.

Because iRNA agent mediated silencing can persist for several days after administering the iRNA agent composition, in many instances, it is possible to administer the composition with a frequency of less than once per day, or, for some instances, only once for the entire therapeutic regimen.

Inhibition of Off-Target Silencing

The sense strand of an iRNA agent can facilitate off-target silencing by hybridizing to a sequence in the genome that belongs to a transcript other than the one desired to be silenced (and therefore different than the transcript bound by the antisense strand of the iRNA agent. To prevent off-target silencing, the sense strand of an iRNA agent can be modified, such as by the addition of a modification (e.g., a chemical or structural modification) to the 5′ or 3′ terminus of the sense strand or to an internal site. Modifications at one or more of these sense strand locations can be useful for inhibiting off-target gene silencing.

In some embodiments, an oligonucleotide or nucleic acid (referred to as “NA” in formulae OT-I through OT-IV below, e.g., RNA, DNA, chimeric RNA-DNA, DNA-RNA, RNA-DNA-RNA, or DNA-RNA-DNA) can be chemically modified by conjugating a moiety that includes a ligand having one or more chemical linkages for attachment of the ligand (L) to the oligonucleotide or nucleic acid. The chemical linkages can include a tether; a chemical linkage between the ligand and the tether (X); a chemical linkage between the tether or ligand and the linker (Y); and/or a chemical linkage between linker, tether or ligand and the oligonucleotide or nucleic acid (Z).

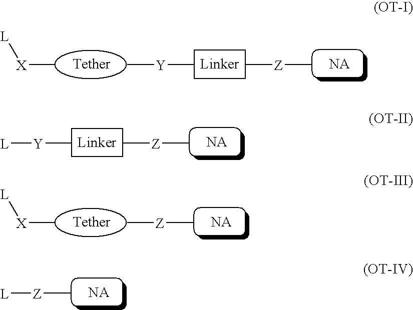

In certain embodiments, an oligonucleotide or nucleic acid can be chemically modified by conjugating one or more moieties having formula OT-I. As shown in Table 1, the moiety can be conjugated to the 3′ or 5′ terminus or an internal position.

TABLE 1

| | |
|---|---|
| 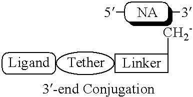 | 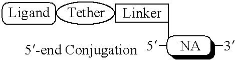 |
| 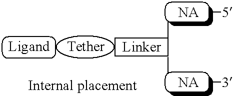 | 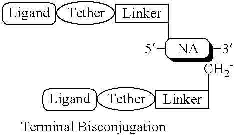 |
| 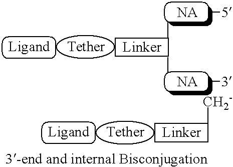 | 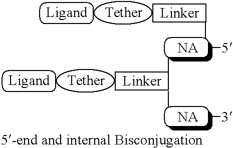 |
| 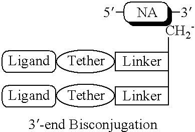 | 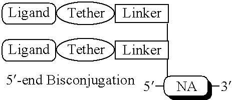 |
| 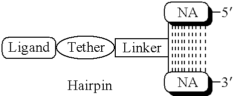 | 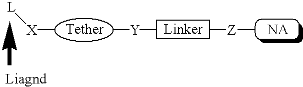 |

In certain embodiments, L can have any one of the values delineated in Table 2.

TABLE 2

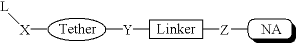

L =
Cholesterol
Thiocholesterol
5β-Cholanic Acid
Cholic acid
Lithocholic acid
Biotin
Vitamin E
Naproxen
Ibuprofen
Amines (mono, di, tri, tetraalkyl or aryl)
Folate
Sugar (N-Acetylgalactosamine, galactosamine, galgactose, Mannose)

—(CH₂)ₙNQ₁Q₂, where n=0-40, Q₁, Q₂=H, Me or Et; Q₁=H, Q₂=H, Me, Et or aryl
—(CH₂)ₚCH═CH(CH₂)_{q}NQ₁Q₂, where p and/or q=0-40, Q₁, Q₂=H, Me or Et; Q₁=H, Q₂=H, Me, Et or aryl with E and/or Z configuration
—(CH₂)ₚCH≡CH(CH₂)_{q}NQ₁Q₂, where p and/or q=0-40, Q₁, Q₂=H, Me or Et; Q₁=H, Q₂=H, Me, Et or aryl
—(CH₂)ₚCH═CH(CH₂)_{q}CH═CH(CH₂)ᵣNQ₁Q₂, where p, q and/or r=0-40, Q₁, Q₂=H, Me or Et; Q₁=H, Q₂=H, Me, Et or aryl with E and/or Z configuration
—O(CH₂)ₘ(OCH₂CH₂)ₙ—OR, where m, n=0-40 and R═H, Me, NQ₁Q₂, —C(O)NR′R″—C(S)NR′R″
—NH(CH₂)ₘ(OCH₂CH₂)ₙ—OR, where m, n=0-40 and R═H, Me, NQ₁Q₂, —C(O)NR′R″—C(S)NR′R″
—O(CH₂)ₘ(NHCH₂CH₂)ₙ—R, where m, n=0-40 and R═H, OH, Me, NQ₁Q₂, —C(O)NR′R″—C(S)NR′R″
—NH(CH₂)ₘ(NHCH₂CH₂)ₙ—R, where m, n=0-40 and R═H, OH, Me, NQ₁Q₂, —C(O)NR′R″—C(S)NR′R″
Dialkylglycerol (sn3, sn1, sn2 and racemic) with number of methylene varies from 0-40
Diacylglycerol (sn3, sn1, sn2 and racemic) with number of methylene varies from 0-40
Dialkylglycerol (sn3, sn1, sn2 and racemic) with number of methylene varies from 0-40 and the alkyl chian contains one or more double bonds with E and/or Z isomers
Diacylglycerol (sn3, sn1, sn2 and racemic) with number of methylene varies from 0-40 and the alkyl chian contains one or more double bonds with E and/or Z isomers
Lipids

In certain embodiments, each of X, Y, and Z can be, independently of one another, any one of the linkages delineated in Table 3.

TABLE 3

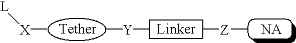

| | | | | | |
|---|---|---|---|---|---|
| X = | —NHC(O)— | Y = | —NHC(O)— | Z = | —NHC(O)— |
| | —C(O)NH— | | —C(O)NH— | | —C(O)NH— |
| | —OC(O)NH— | | —OC(O)NH— | | —OC(O)NH— |
| | —NHC(O)O— | | —NHC(O)O— | | —NHC(O)O— |
| | —O— | | —O— | | —O— |
| | —S— | | —S— | | —S— |
| | —SS— | | —SS— | | —SS— |
| | —S(O)— | | —S(O)— | | —S(O)— |
| | —S(O₂)— | | —S(O₂)— | | —S(O₂)— |
| | —NHC(O)NH— | | —NHC(O)NH— | | —NHC(O)NH— |
| | —NHC(S)NH— | | —NHC(S)NH— | | —NHC(S)NH— |
| | —C(O)O— | | —C(O)O— | | —C(O)O— |
| | —OC(O)— | | —OC(O)— | | —OC(O)— |
| | —NHC(S)— | | —NHC(S)— | | —NHC(S)— |
| | —NHC(S)O— | | —NHC(S)O— | | —NHC(S)O— |
| | —C(S)NH— | | —C(S)NH— | | —C(S)NH— |
| | —OC(S)NH— | | —OC(S)NH— | | —OC(S)NH— |
| | —NHC(S)O— | | —NHC(S)O— | | —NHC(S)O— |
| | —CH₂— | | —CH₂— | | —CH₂— |
| | —CH₂CH═CH— | | —CH₂CH═CH— | | —CH₂CH═CH— |
| | —C(O)CH═CH— | | —C(O)CH═CH— | | —C(O)CH═CH— |
| | —NH—CH₂CH═CH— | | —NH—CH₂CH═CH— | | —NH—CH₂CH═CH— |
| | —O—P(O)(OH)—O— | | —O—P(O)(OH)—O— | | —O—P(O)(OH)—O— |
| | —O—P(S)(OH)—O— | | —O—P(S)(OH)—O— | | —O—P(S)(OH)—O— |
| | —O—P(S)(SH)—O— | | —O—P(S)(SH)—O— | | —O—P(S)(SH)—O— |
| | —S—P(O)(OH)—O— | | —S—P(O)(OH)—O— | | —S—P(O)(OH)—O— |
| | —O—P(O)(OH)—S— | | —O—P(O)(OH)—S— | | —O—P(O)(OH)—S— |
| | —S—P(O)(OH)—S— | | —S—P(O)(OH)—S— | | —S—P(O)(OH)—S— |
| | —O—P(S)(OH)—S— | | —O—P(S)(OH)—S— | | —O—P(S)(OH)—S— |
| | —S—P(S)(OH)—O— | | —S—P(S)(OH)—O— | | —S—P(S)(OH)—O— |
| | —O—P(O)(R)—O— | | —O—P(O)(R)—O— | | —O—P(O)(R)—O— |
| | —O—P(S)(R)—O— | | —O—P(S)(R)—O— | | —O—P(S)(R)—O— |
| | —S—P(O)(R)—O— | | —S—P(O)(R)—O— | | —S—P(O)(R)—O— |
| | —S—P(S)(R)—O— | | —S—P(S)(R)—O— | | —S—P(S)(R)—O— |
| | —S—P(O)(R)—S— | | —S—P(O)(R)—S— | | —S—P(O)(R)—S— |
| | —O—P(S)(R)—S— | | —O—P(S)(R)—S— | | —O—P(S)(R)—S— |

R = Alkyl, fluroalkyl, aryl or aralkyl

In certain embodiments, the tether can have any one of the values delineated in Table 4. Off-target gene silencing can be inhibited by a variety of mechanisms. For example,

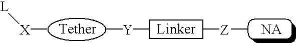

| | Linker = | | |
|---|---|---|---|
| Tether: | 3′-end | 5′-end | interior |
| —(CH₂)ₙ—, where n = 1-40 —(CH₂—CH₂O)ₙ—, where n = 1-20 —O(CH₂—CH₂O)ₙ—, where n = 1-20 —(CH₂—CH₂NH)ₙ—, where n = 1-20 —NH(CH₂—CH₂NH)ₙ—, where n = 1-20 | 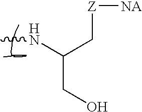 | 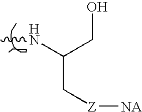 | 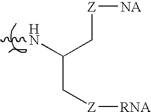 |
| —(CH₂)ₗ[(CH═CH)ₘ(CH₂)ₙ]ₚ(CH═CH)_{q}(CH₂)ᵣ—, where l, m, n, p, q and/or r = 0-20 —(CH₂)ₗ[(C≡C)ₘ(CH₂)ₙ]ₚ(C≡C)_{q}(CH₂)ᵣ—, where | | | |
| #l, m, n, p, q and/or r = 0-20 | 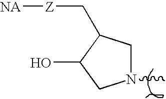 | 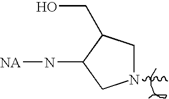 | 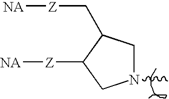 |
| | 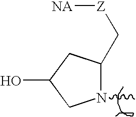 | 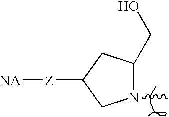 | 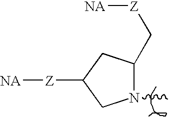 |
| | 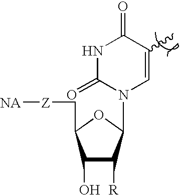 | 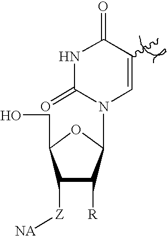 | 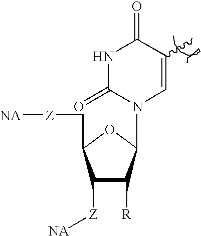 |
| | 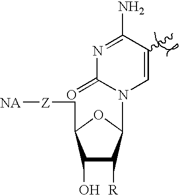 | 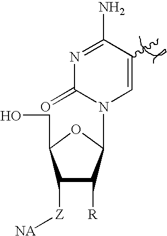 | 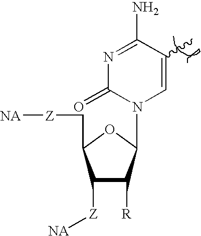 |
| | | 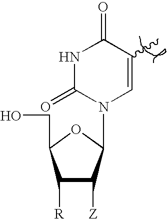 | 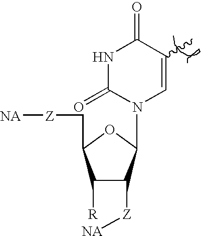 |
| | | 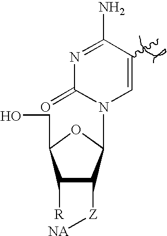 | 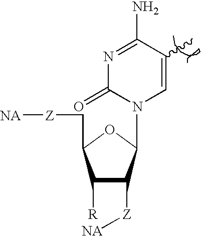 |

hydrolysis of the 5′-phosphate group of the sense strand of an iRNA agent may facilitate the assembly of the sense strand into the RISC complex, and a subsequent silencing activity of an off-target transcript. Modifications that block this hydrolysis of the 5′-phosphate group may prevent this assembly step, thereby preventing silencing activity by the sense strand. For example, placement of one or more L-nucleosides, alpha-nucleosides, or 2′-5′ linkages onto the 5′ end of the sense strand may prevent hydrolysis. In one alternative hypothesis, modifications that sufficiently alter the shape or size or charge of the 5′ terminus may prevent entry into the RISC complex, or may prevent off-target gene silencing by an as yet undiscovered mechanism.

In certain embodiments, off target gene silencing can be inhibited by 5′-end capping of a sense strand, e.g., with D-nucleosides, L-nucleosides, α-nucleosides, or L-α-nucleosides (see Tables 5, 6, 7, and 8).

TABLE 5

| | |
|---|---|
| 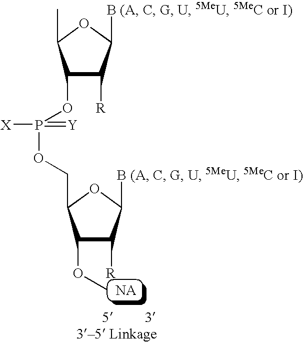 | 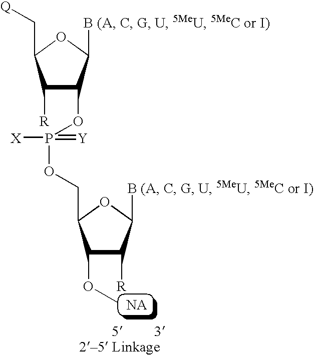 |

R = H, OH, F, O(CH₂)ₙMe, O(CH₂)ₙOMe, O(CH₂)ₙNMe₂, O(CH₂)ₙCONH₂, O(CH₂)ₙONH₂, O(CH₂)ₙNH₂, NHMe,
NMe₂, NH₂, NHAc, N(Me)Ac, O(CH₂)ₙCONHMe, O(CH₂)ₙONHMe, O[(CH₂)ₙO]ₘMe, O[(CH₂)ₙO]ₘNH₂,
O[(CH₂)ₙO]ₘNHAc, O(CH₂)ₙNMeAc, O[(CH₂)ₙO]ₘ(CH₂)ₗNH₂, O(CH₂)ₙONMeAc, O(CH₂)ₙONMe₂, O(CH₂)ₙSMe,
O(CH₂)ₙS(O)Me, O(CH₂)ₙS(O₂)Me, where, l, m, n, = 0-40
X = OH, SH, Me, Et, ispropyl, t-butyl, CH₂F, CHF₂, CF₃, phenyl, benzyl and Y = O or S

Q=
NH2, NHMe, NMe2, (CH2)nH, (CH2)nQ′R′, where Q′=O or S R′ is H, (CH2)nH, aryl or aralkyl
(CH2)nS(O)R′, (CH2)nS(O2)R″where R′ is H, (CH2)nH, aryl or aralkyl
Q′(CH2)nS(O)R″, Q′(CH2)nS(O2)R″, where Q′=O, S, S—S, S(O), S(O2), CH2 or NR′ and R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl and/or alkynyl
Q′(CH2)nNR′R″, where X═O, S, S—S, S(O), S(O2), CH2 or NR′ and R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl, acetyl and and/or acyl
Q′(CH2)nQ″R″, where Q′=O, S, S—S, S(O), S(O2), CH2 or NR′; Q″ is O, S, S(O), S(O2), CH2 and R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl, acetyl and/or acyl
Q′(CH2CH2O)nNR′R″, where Q′=O, S, S—S, S(O), S(O2), CH2 or NR′ and R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl, acetyl and/or acyl
Q′(CH2CH2O)nQ″R″, where Q′=O, S, S—S, S(O), S(O2), CH2 or NR′; Q″ is O, S, S(O), S(O2), CH2, NR′ or H and R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl, acetyl and/or acyl
Q′(CH2CH2O)n(CH2)mQ″R″, where Q′=O, S, S—S, S(O), S(O2), CH2 or NR′; Q″ is O, S, S(O), S(O2), CH2, NR′ or H and R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl, acetyl and/or acyl
Q′(CH2CH2NH)n(CH2)mQ″R″, where Q′=O, S, S—S, S(O), S(O2), CH2 or NR′; Q″ is O, S, S(O), S(O2), CH2, NR′ or H and R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl, acetyl and/or
OC(Q′)NR′R″, where Q is O, S or NH; R′ are R″, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl, acetyl and/or acyl
C(Q′)NR′R″, where Q′ is O, S or NH; R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl and/or acyl
NR′C(Q′)NR′R″, where Q′ is O, S or NH; R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl and/or acyl
NR′C(Q′)R″, where Q is O, S or NH; R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl and/or acyl
NR′C(Q′)OR″, where Q′ is O or A; R′ and R″ are H, alkyl, aryl, aminoalkyl, alkylaminoalkyl, dialkylaminoalkyl, hydroxyalkyl, carboxyalkyl, mercaptoalkyl, aralkyl, alicyclic, alkenyl, alkynyl and/or acyl
   l, n, m=0-40

TABLE 6
5′-end capping of sense strand with L-nucleosides

| | |
|---|---|
| 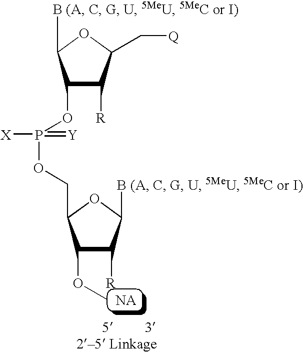 | 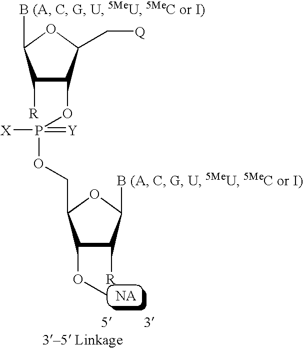 |

R = H, OH, F, O(CH₂)ₙMe, O(CH₂)ₙOMe, O(CH₂)ₙNMe₂, O(CH₂)ₙCONH₂, O(CH₂)ₙONH₂, O(CH₂)ₙNH₂, NHMe,
NMe₂, NH₂, NHAc, N(Me)Ac, O(CH₂)ₙCONHMe, O(CH₂)ₙONHMe, O[(CH₂)ₙO]ₘMe, O[(CH₂)ₙO]ₘNH₂,
O[(CH₂)ₙO]ₘNHAc, O(CH₂)ₙNMeAc, O[(CH₂)ₙO]ₘ(CH₂)ₗNH₂, O(CH₂)ₙONMeAc, O(CH₂)ₙONMe₂, O(CH₂)ₙSMe,
O(CH₂)ₙS(O)Me, O(CH₂)ₙS(O₂)Me, where, l, m, n = 0-40
X = OH, SH, Me, Et, isopropyl, t-butyl, CH₂F, CHF₂, CF₃, phenyl, benzyl and Y = O or S
Q =
NH₂, NHMe, NMe₂, (CH₂)ₙH, (CH₂)ₙQ′R′, where Q′ = O or S R′ is H, (CH₂)ₙH, aryl or aralkyl
(CH₂)ₙS(O)R′, (CH₂)ₙS(O₂)R′, where R′ is H, (CH₂)ₙH, aryl or aralkyl
Q′(CH₂)ₙS(O)R″, Q′(CH₂)ₙS(O₂)R″, where Q′ = O, S, S—S, S(O), S(O₂), CH₂ or NR′ and R′ and R″ are H,
alkyl, aryl, aminoalkyl, alkylaminoalkyl, dialkylaminoalkyl, hydroxyalkyl, carboxyalkyl, mercaptoalkyl,
aralkyl, alicyclic, alkenyl and/or alkynyl
Q′(CH₂)ₙNR′R″, where X = O, S, S—S, S(O), S(O₂), CH₂ or NR′ and R′ and R″ are H, alkyl, aryl,
aminoalkyl, alkylaminoalkyl, dialkylaminoalkyl, hydroxyalkyl, carboxyalkyl, mercaptoalkyl, aralkyl, alicyclic,
alkenyl, alkynyl, acetyl and and/or acyl
Q′(CH₂)ₙQ″R″, where Q′ = O, S, S—S, S(O), S(O₂), CH₂ or NR′; Q″ is O, S, S(O), S(O₂), CH₂ and R′ and
R″ are H, alkyl, aryl, aminoalkyl, alkylaminoalkyl, dialkylaminoalkyl, hydroxyalkyl, carboxyalkyl,
mercaptoalkyl, aralkyl, alicyclic, alkenyl, alkynyl, acetyl and/or acyl
Q′(CH₂CH₂O)ₙNR′R″, where Q′ = O, S, S—S, S(O), S(O₂), CH₂ or NR′ and R′ and R″ are H, alkyl, aryl,
aminoalky, alkylaminoalky, dialkylaminoalky, hydroxyalkyl, carboxyalkyl, mercaptoalkyl, aralkyl, alicyclic,
alkenyl, alkynyl, acetyl and/or acyl
Q′(CH₂CH₂O)ₙQ″R″, where Q′ = O, S, S—S, S(O), S(O₂), CH₂ or NR′; Q″ is O, S, S(O), S(O₂), CH₂, NR′
or H and R′ and R″ are H, alky, aryl, aminoalkyl, alkylaminoalkyl, dialkylaminoalkyl, hydroxyalkyl,
carboxylalkyl, mercaptoalkyl, aralkyl, alicyclic, alkenyl, alkynyl, acetyl and/or acyl
Q′(CH₂CH₂O)ₙ(CH₂)ₘQ″R″, where Q′ = O, S, S—S, S(O), S(O₂), CH₂ or NR′; Q″ is O, S, S(O), S(O₂),
CH₂, NR′ or H and R′ and R″ are H, alkyl, aryl, aminoalkyl, alkylaminoalkyl, dialkylaminoalkyl,
hydroxyalkyl, carboxyalkyl, mercaptoalkyl, aralkyl, alicyclic, alkenyl, alkynyl, acetyl and/or acyl
Q′(CH₂CH₂NH)ₙ(CH₂)ₘQ″R″, where Q′ = O, S, S—S, S(O), S(O₂), CH₂ or NR′; Q″ is O, S, S(O), S(O₂),
CH₂, NR′ or H and R′ and R″ are H, alkyl, aryl, aminoalkyl, alkylaminoalkyl, dialkylaminoalkyl,
hydroxyalkyl, carboxylalkyl, mercaptoalkyl, aralkyl, alicyclic, alkenyl, alkynyl, acetyl and/or
OC(Q′)NR′R″, where Q is O, S or NH; R′ are R″, alkyl, aryl, aminoalkyl, alkylaminoalkyl, dialkylaminoalkyl,
hydroxyalkyl, carboxyalkyl, mercaptoalkyl, aralkyl, alicyclic, alkenyl, alkynyl, acetyl and/or acyl
C(Q′)NR′R″, where Q′ is O, S or NH; R′ and R″ are H, alkyl aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
NR′C(Q′)NR′R″, where Q′ is O, S or NH; R′ and R″ are H, alkyl, aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
NR′C(Q′)R″, where Q is O, S or NH; R′ and R″ are H, alkyl, aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
NR′C(Q′)OR″, where Q′ is O or A; R′ and R″ are H, alkyl, aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
l, n, m = 0-40

TABLE 7
Preferred sugar modification in Sense and in Antisense strands

| Sense strand | Antisense strand |
|---|---|
| 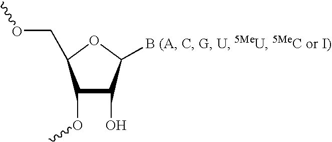 | 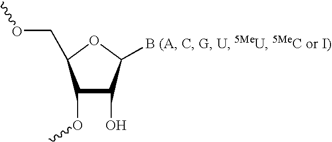 |
| 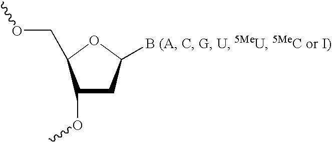 | 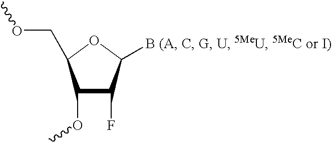 |
| 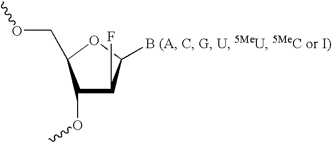 | 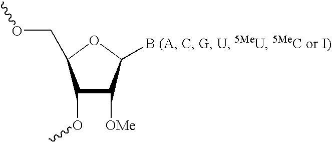 |
| 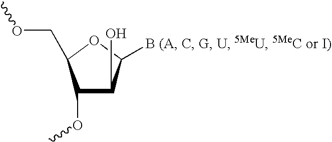 | 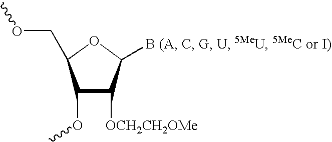 |
| 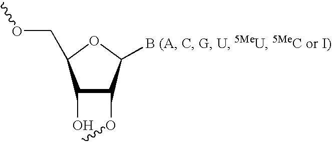 | 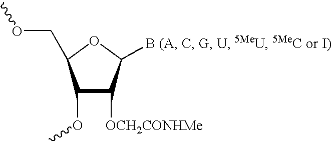 |
| 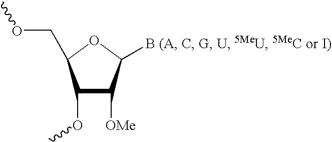 | |
| 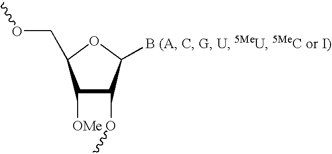 | |
| 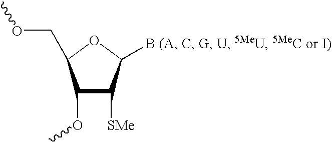 | |
| 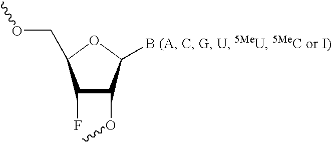 | |
| 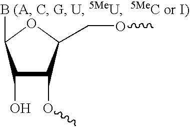 | |
| 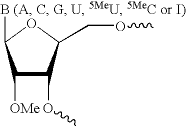 | |
| 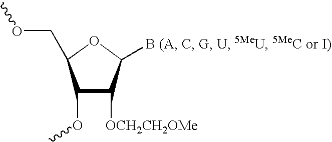 | |
| 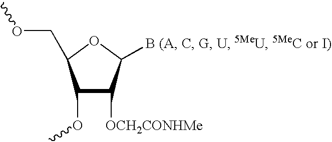 | |

TABLE 8
5′-End capping of sense strand with α-nucleosides

| | |
|---|---|
| 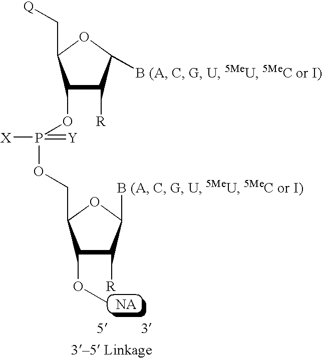 | 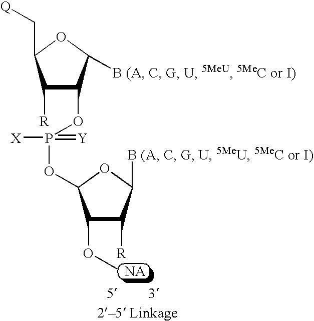 |

R = H, OH, F, O(CH₂)ₙMe, O(CH₂)ₙOMe, O(CH₂)ₙNMe₂, O(CH₂)ₙCONH₂, O(CH₂)ₙONH₂, O(CH₂)ₙNH₂, NHMe,
NMe₂, NH₂, NHAc, N(Me)Ac, O(CH₂)ₙCONHMe, O(CH₂)ₙONHMe, O[(CH₂)ₙO]ₘMe, O[(CH₂)ₙO]ₘNH₂,
O[(CH₂)ₙO]ₘNHAc, O(CH₂)ₙNMeAc, O[(CH₂)ₙO]ₘ(CH₂)ₗNH₂, O(CH₂)ₙONMeAc, O(CH₂)ₙONMe₂, O(CH₂)ₙSMe,
O(CH₂)ₙS(O)Me, O(CH₂)ₙS(O₂)Me, where, l, m, n = 0-40
X = OH, SH, Me, Et, isopropyl, t-butyl, CH₂F, CHF₂, CF₃, phenyl, benzyl and Y = O or S
Q =
NH₂, NHMe, NMe₂, (CH₂)ₙH, (CH₂)ₙQ′R′, where Q′ = O or S R′ is H, (CH₂)ₙH, aryl or aralkyl
(CH₂)ₙS(O)R′, (CH₂)ₙS(O₂)R′, where R′ is H, (CH₂)ₙH, aryl or aralkyl
Q′(CH₂)ₙS(O)R″, Q′(CH₂)ₙS(O₂)R″, where Q′ = O, S, S—S, S(O), S(O₂), CH₂ or NR′ and R′ and R″ are H,
alkyl, aryl, aminoalkyl, alkylaminoalkyl, dialkylaminoalkyl, hydroxyalkyl, carboxyalkyl, mercaptoalkyl,
aralkyl, alicyclic, alkenyl and/or alkynyl
Q′(CH₂)ₙNR′R″, where X = O, S, S—S, S(O), S(O₂), CH₂ or NR′ and R′ and R″ are H, alkyl, aryl,
aminoalkyl, alkylaminoalkyl, dialkylaminoalkyl, hydroxyalkyl, carboxyalkyl, mercaptoalkyl, aralkyl, alicyclic,
alkenyl, alkynyl, acetyl and and/or acyl
Q′(CH₂)ₙQ″R″, where Q′ = O, S, S—S, S(O), S(O₂), CH₂ or NR′; Q″ is O, S, S(O), S(O₂), CH₂ and R′ and
R″ are H, alkyl, aryl, aminoalkyl, alkylaminoalkyl, dialkylaminoalkyl, hydroxyalkyl, carboxyalkyl,
mercaptoalkyl, aralkyl, alicyclic, alkenyl, alkynyl, acetyl and/or acyl
Q′(CH₂CH₂O)ₙNR′R″, where Q′ = O, S, S—S, S(O), S(O₂), CH₂ or NR′ and R′ and R″ are H, alkyl, aryl,
aminoalkyl, alkylaminoalkyl, dialkylaminoalkyl, hydroxyalkyl, carboxyalkyl, mercaptoalkyl, aralkyl, alicyclic,
alkenyl, alkynyl, acetyl and/or acyl
Q′(CH₂CH₂O)ₙQ″R″, where Q′ = O, S, S—S, S(O), S(O₂), CH₂ or NR′; Q″ is O, S, S(O), S(O₂), CH₂, NR′
or H and R′ and R″ are H, alkyl, aryl, aminoalkyl, alkylaminoalkyl, dialkylaminoalkyl, hydroxyalkyl,
carboxyalkyl, mercaptoalkyl, aralky, alicyclic, alkenyl, alkynyl, acetyl and/or acyl
Q′(CH₂CH₂O)ₙ(CH₂)ₘQ″R″, where Q′ = O, S, S—S, S(O), S(O₂), CH₂ or NR′; Q″ is O, S, S(O), S(O)₂,
CH₂, NR′ or H and R′ and R″ are H, alkyl, aryl, aminoalkyl, alkylaminoalkyl, dialkylaminoalkyl,
hydroxyalkyl, carboxyalkyl, mercaptoalkyl, aralkyl, alicyclic, alkenyl, alkynyl, acetyl and/or
Q′(CH₂CH₂NH)ₙ(CH₂)ₘQ″R″, where Q′ = O, S, S—S, S(O), S(O₂), CH₂ OR NR′; Q″ is O, S, S(O), S(O)₂,
CH₂, NR′ or H and R′ and R″ are H, alkyl, aryl, aminoalkyl, alkylaminoalkyl, dialkylaminoalkyl,
hydroxyalkyl, carboxyalkyl, mercaptoalkyl, aralkyl, alicyclic, alkenyl, alkynyl, acetyl and/or
OC(Q′)NR′R″, where Q is O, S or NH; R′ are R″, alkyl, aryl, aminoalkyl, alkylaminoalkyl, dialkylaminoalkyl,
hydroxyalkyl, carboxyalkyl, mercaptoalkyl, aralkyl, alicyclic, alkenyl, alkynyl, acetyl and/or acyl
C(Q′)NR′R″, where Q′ is O, S or NH; R′ and R″ are H, alkyl, aryl, aminoalkyl, alkylaminoalkyl,
dialkylaminoalkyl, hydroxyalky, carboxyalkyl, mercaptoalkyl, aralkyl, alicyclic, alkenyl, alkynyl and/or acyl
NR′C(Q′)NR′R″, where Q′ is O, S or NH; R′ and R″ are H, alkyl, aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
NR′C(Q′)R″, where Q is O, S or NH; R′ and R″ are H, alkyl, aryl, aminoalkyl, alkylaminoalkyl,
dialkylaminoalkyl, hydroxyalkyl, carboxylalkyl, mercaptoalkyl, aralkyl, alicyclic, alkenyl, alkynyl and/or acyl
NR′C(Q′)OR″, where Q′ is O or A; R′ and R″ are H, alkyl, aryl, aminoalkyl, alkylaminoalkyl,
dialkylaminoalkyl, hydroxyalkyl, carboxylalkyl, mercaptoalkyl, aralkyl, alicyclic, alkenyl, alkynyl and/or acyl
l, n, m = 0-40

TABLE 9
5′-end capping of sense strand with L-α-nucleosides

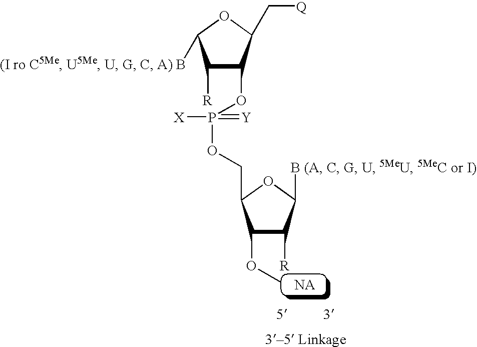

R = H, OH, F, O(CH₂)ₙMe, O(CH₂)ₙOMe, O(CH₂)ₙNMe₂, O(CH₂)ₙCONH₂, O(CH₂)ₙONH₂, O(CH₂)ₙNH₂, NHMe,
NMe₂, NH₂, NHAc, N(Me)Ac, O(CH₂)ₙCONHMe, O(CH₂)ₙONHMe, O[(CH₂)ₙO]ₘMe, O[(CH₂)ₙO]ₘNH₂,
O[(CH₂)ₙO]ₘNHAc, O(CH₂)ₙNMeAc, O[(CH₂)ₙO]ₘ(CH₂)ₗNH₂, O(CH₂)ₙONMeAc, O(CH₂)ₙONMe₂, O(CH₂)ₙSMe
O(CH₂)ₙS(O)Me, O(CH₂)ₙS(O₂)Me, where l, m, n = 0-40
X = OH, SH, Me, Et, isopropyl, t-butyl, CH₂F, CHF₂, CF₃, phenyl, benzyl and Y = O or S
Q =
NH₂, NHMe, NMe₂, (CH₂)ₙH, (CH₂)ₙQ′R′, where Q′ = O or S R′ is H, (CH₂)ₙH, aryl or aralkyl
(CH₂)ₙS(O)R′, (CH₂)ₙS(O₂)R′, where R′ is H, (CH₂)ₙH, aryl or aralkyl
Q′(CH₂)ₙS(O)R″, Q′(CH₂)ₙS(O₂)R″, where Q′ = O, S, S—S, S(O), S(O₂), CH₂ or NR′ and R′ and R″ are H,
alkyl, aryl, aminoalkyl, alkylaminoalkyl, dialkylaminoalkyl, hydroxyalkyl, carboxyalkyl, mercaptoalkyl,
aralkyl, alicyclic, alkenyl and/or alkynyl
Q′(CH₂)ₙNR′R″, where X = O, S, S—S, S(O), S(O₂), CH₂ or NR′ and R′ and R″ are H, alkyl, aryl,
aminoalkyl, alkylaminoalkyl, dialkylaminoalkyl, hydroxyalkyl, carboxyalkyl, mercaptoalkyl, aralkyl, alicyclic,
alkenyl, alkynyl, acetyl and and/or a acyl
Q′(CH₂)ₙQ″R″, where Q′ = O, S, S—S, S(O), S(O₂), CH₂ or NR′; Q″ is O, S, S(O), S(O₂), CH₂ and R′ and
R″ are H, alkyl, aryl, aminoalkyl, alkylaminoalkyl, dialkylaminoalkyl, hydroxyalkyl, carboxyalkyl,
mercaptoalkyl, aralkyl, alicyclic, alkenyl, alkynyl, acetyl and/or acyl
Q′(CH₂CH₂O)ₙNR′R″, where Q′ = O, S, S—S, S(O), S(O₂), CH₂ or NR′ and R′ and R″ are H, alkyl, aryl,
aminoalkyl, alkylaminoalkyl, dialkylaminoalkyl, hydroxyalkyl, carboxyalkyl, mercaptoalkyl, aralkyl, alicyclic,
alkenyl, alkynyl, acetyl and/or acyl
Q′(CH₂CH₂O)ₙQ″R″, where Q′ = O, S, S—S, S(O), S(O₂), CH₂ or NR′; Q″ is O, S, S(O), S(O₂), CH₂, NR′
or H and R′ and R″ are H, alkyl, aryl, aminoalkyl, alkylaminoalkyl, dialkylaminoalkyl, hydroxyalkyl,
carboxyalkyl, mercaptoalkyl, aralky, alicyclic, alkenyl, alkynyl, acetyl and/or acyl
Q′(CH₂CH₂O)ₙ(CH₂)ₘQ″R″, where Q′ = O, S, S—S, S(O), S(O₂), CH₂ or NR′; Q″ is O, S, S(O), S(O₂),
CH₂, NR′ or H and R′ and R″ are H, alkyl, aryl, aminoalkyl, alkylaminoalkyl, dialkylaminoalkyl,
hydroxyalkyl, carboxyalkyl, mercaptoalkyl, aralkyl, alicyclic, alkenyl, alkynyl, acetyl and/or acyl
Q′(CH₂CH₂NH)ₙ(CH₂)ₘQ″R″, where Q′ = O, S, S—S, S(O), S(O₂), CH₂ or NR′; Q″ is O, S, S(O), S(O₂),
CH₂, NR′ or H and R′ and R″ are H, alkyl, aryl, aminoalkyl, alkyaminoalkyl, dialkylaminoalkyl,
hydroxyalkyl, carboxyalkyl, mercaptoalkyl, aralky, alicyclic, alkenyl, alkynyl, acetyl and/or
OC(Q′)NR′R″, where Q is O, S or NH; R′ are R″, alkyl, aryl, aminoalkyl, alkylaminoalkyl, dialkylaminoalkyl,
hydroxyalkyl, carboxyalkyl, mercaptoalkyl, aralkyl, alicyclic, alkenyl, alkynyl, acetyl and/or acyl
C(Q′)NR′R″, where Q′ is O, S or NH; R′ and R″ are H, alkyl, aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
NR′C(Q′)NR′R″, where Q′ is O, S or NH; R′ and R″ are H, alkyl, aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
NR′C(Q′)R″, where Q IS O, S or NH; R′ and R″ are H, alkyl, aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
NR′C(Q′)OR″, where Q′ is O or A; R′ and R″ are H, alkyl, aryl, aminoalkyl, alkylaminoalkyl,
dialkylaminoalkyl, hydroxyalkyl, carboxyalkyl, mercaptoalkyl, aralkyl, alicyclic, alkenyl, alkynyl and/or acyl
l, n, m = 0-40

Compounds described herein can be prepared by methods described herein or by conventional methods from commercially available reagents and starting materials.

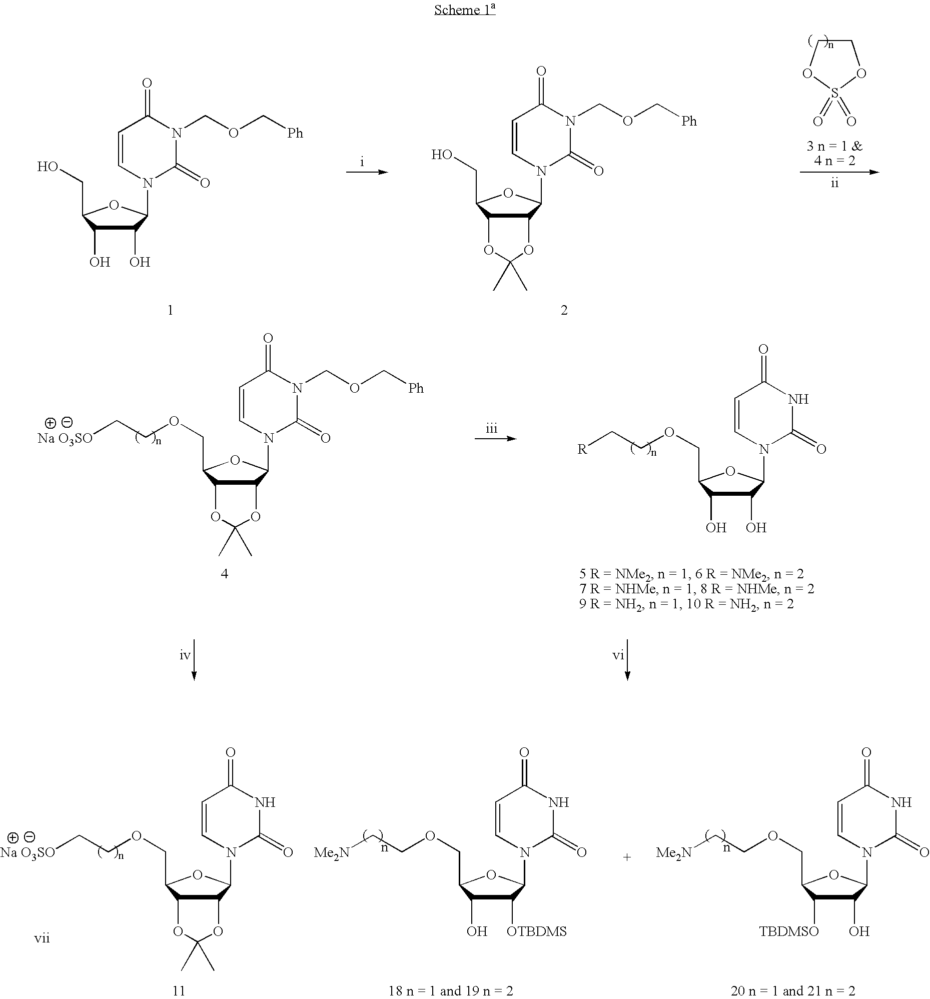

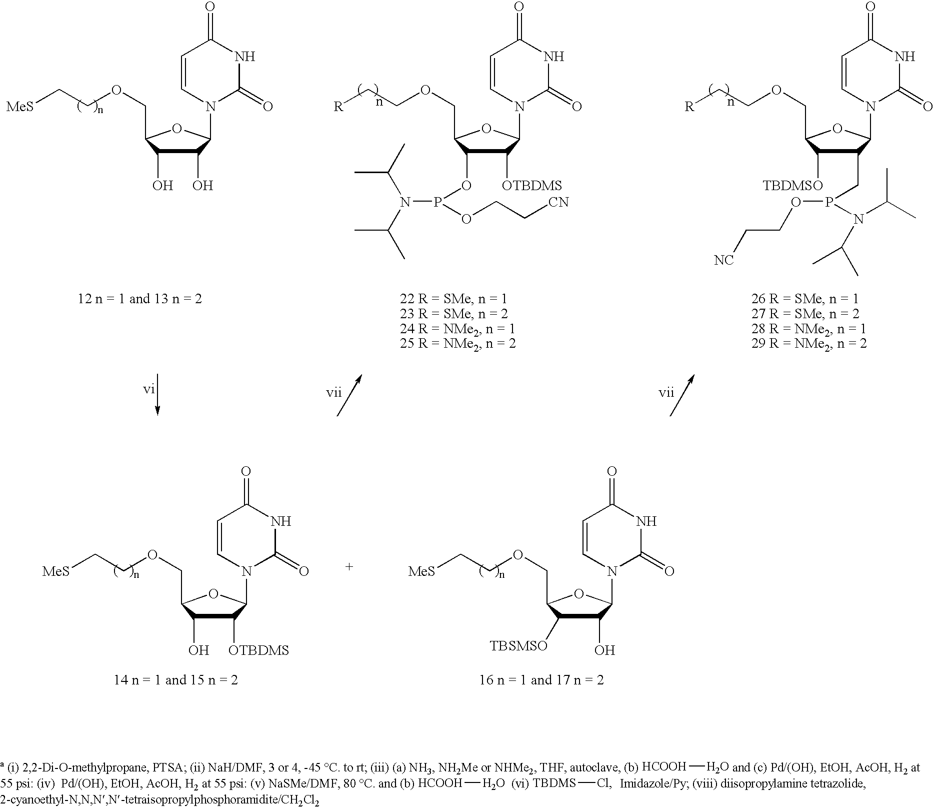

Compound 1 is prepared as reported by Fraser et al. (Tetrahedron Lett., 2000, 41, 1523). Steps (ii), (iii) (a), (iii) (c), (iv), (v) and (vii) are performed according to literature procedure (Fraser et al., Tetrahedron Lett., 2000, 41, 1523). Step (iii) (b) and (v) (b) are performed as reported in the literature (Bioorg. Med. Chem. Lett., 2003, 13, 1713). Step (iv) is performed as reported in the literature (Corey and Venkateswarlu, J. Am. Chem. Soc., 1972, 94, 6190).

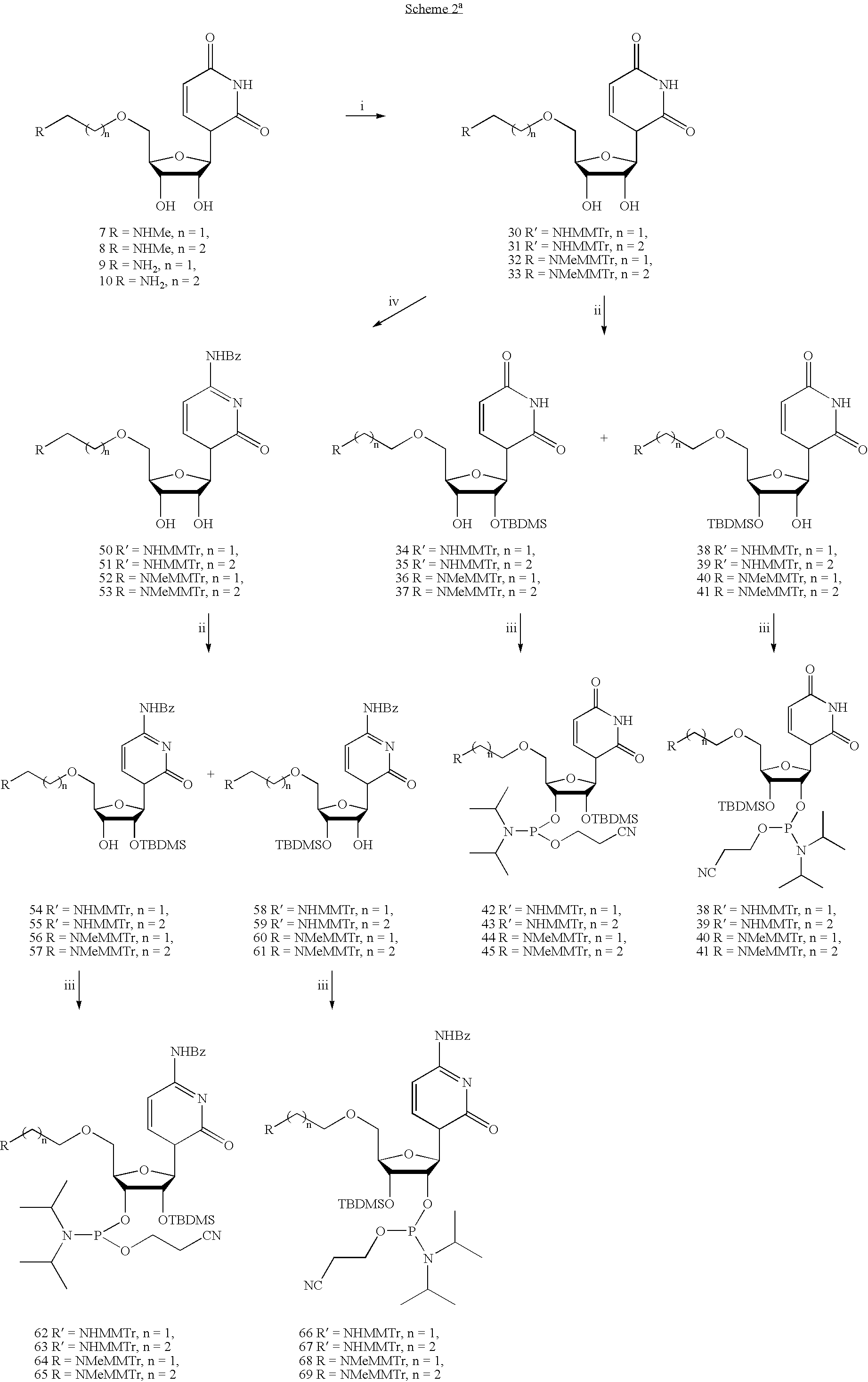

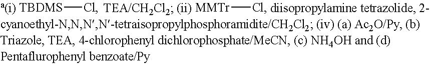

Step (i) is performed as reported in the literature (Dubowchik and Radia, Tetrahedron Lett., 1997, 38, 5257); step (ii) is performed as reported in the literature (Corey and Venkateswarlu, J. Am. Chem. Soc., 1972, 94, 6190); step (iii) is performed as reported by Fraser et al. (Tetrahedron Lett., 2000, 41, 1523) and step (iv) is performed by reported procedures (Miller et al., Current Protocol in Nucleic Acids Chemistry, 2000, 2.5.1-2.5.36, (John Wiley and Sons, Inc.).

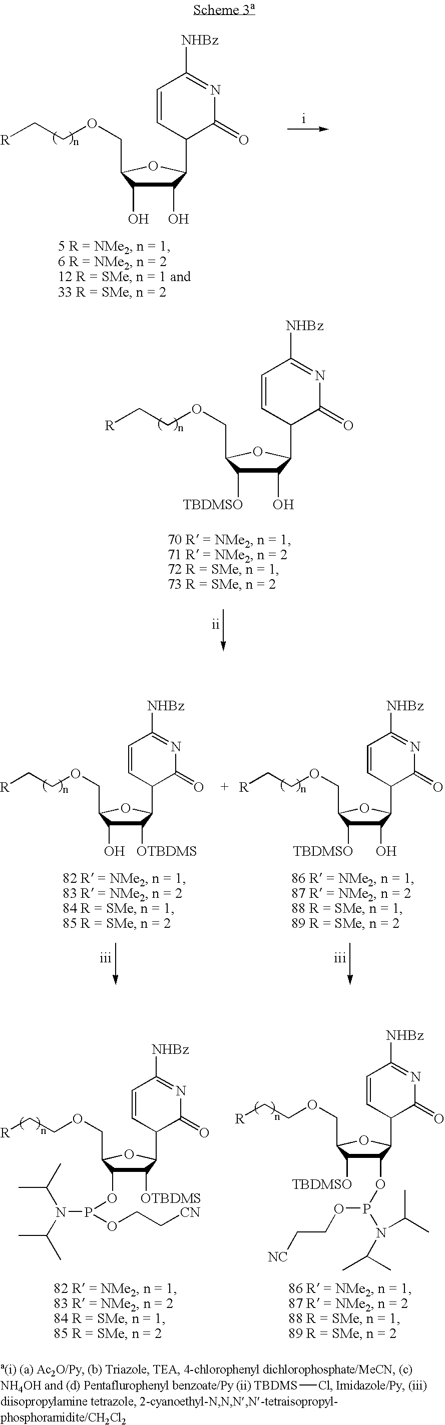

Step (i) is performed by reported procedures (Miller et al., Current Protocol in Nucleic Acids Chemistry, 2000, 2.5.1-2.5.36, (John Wiley and Sons, Inc.); step 2 is performed as reported in the literature (Corey and Venkateswarlu, J. Am. Chem. Soc., 1972, 94, 6190) and step (iii) is performed as reported by Fraser et al. (Tetrahedron Lett., 2000, 41, 1523).

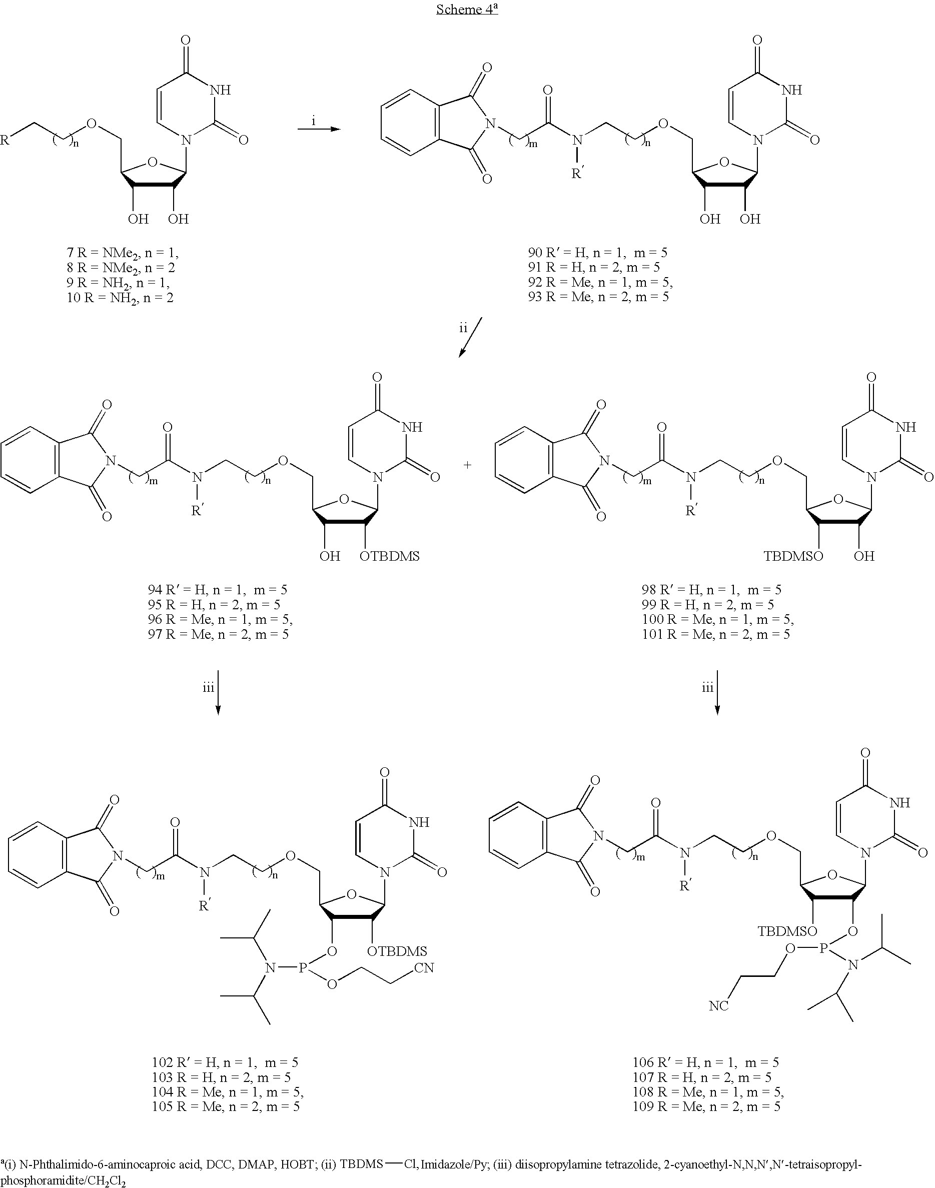

Step 2 is performed as reported in the literature (Corey and Venkateswarlu, J. Am. Chem. Soc., 1972, 94, 6190) and step (iii) is performed as reported by Fraser et al. (Tetrahedron Lett., 2000, 41, 1523).

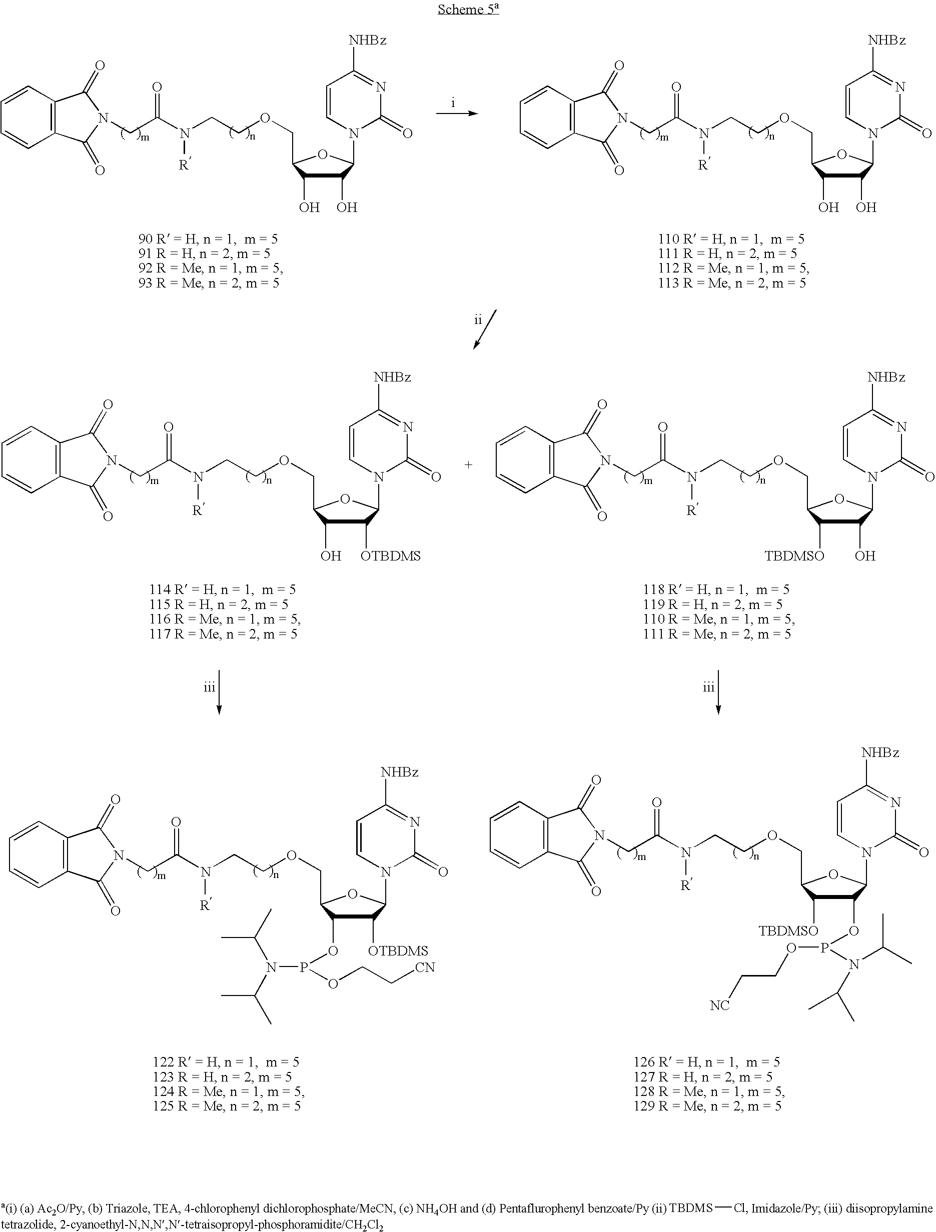

Step (i) is performed by reported procedures (Miller et al., Current Protocol in Nucleic Acids Chemistry, 2000, 2.5.1-2.5.36, (John Wiley and Sons, Inc.); step (ii) is performed as reported in the literature (Corey and Venkateswarlu, J. Am. Chem. Soc., 1972, 94, 6190) and step (iii) is performed as reported by Fraser et al. (Tetrahedron Lett., 2000, 41, 1523).

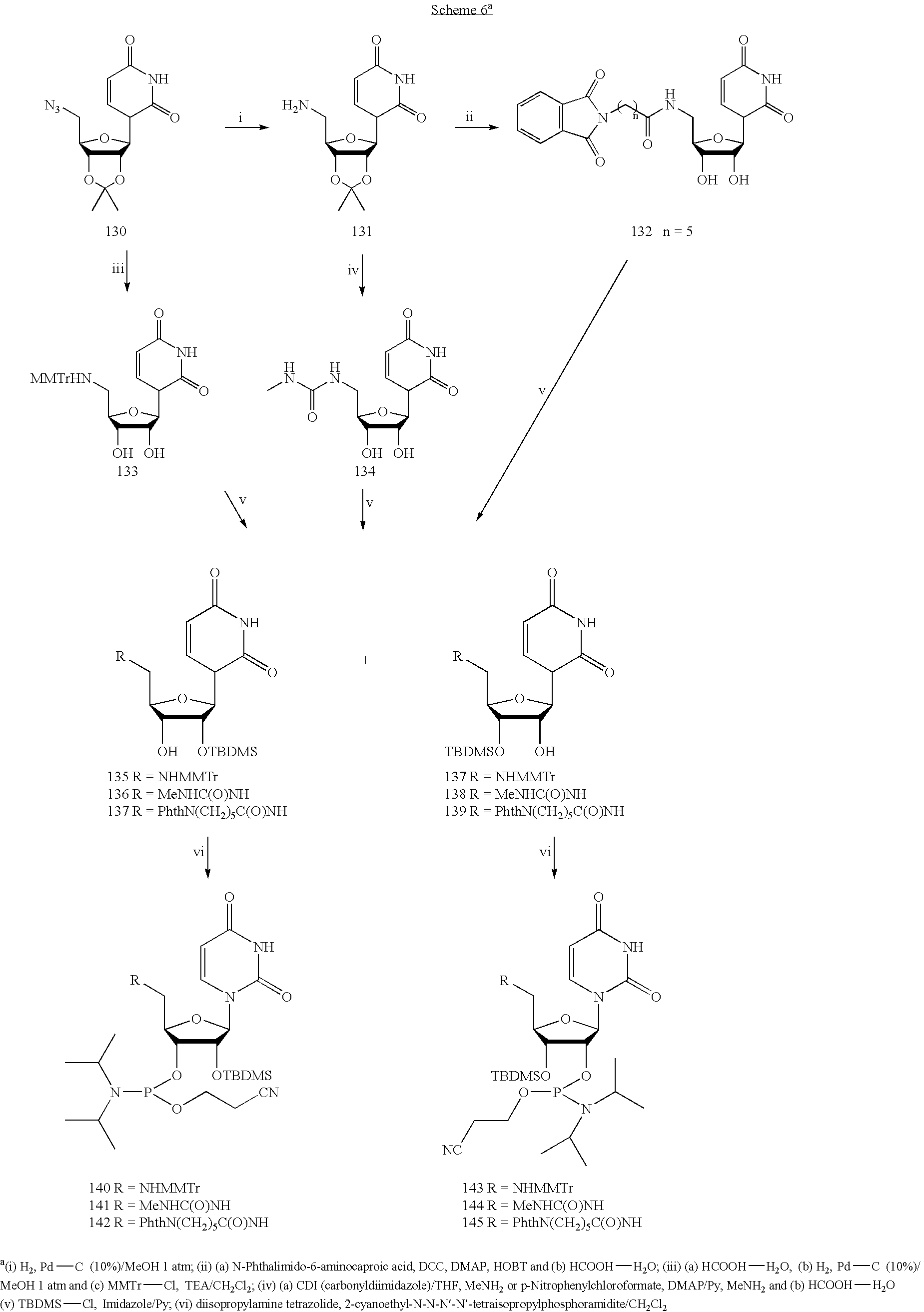

Compound 130 is obtained as reported in the literature (Liu and Austin, J. Org. Chem., 2001, 66, 8643). Step (i) and (iii) (b) are performed as reported in the literature (Chem. Rev., 1954, 54, 1); step (ii) (a) is performed according to literature procedures (J. Org. Chem., 1993, 58, 2334); step (ii) (b), (iii) (a) and (iv) (b) are performed as reported in the literature (Bioorg. Med. Chem. Lett., 2003, 13, 1713); step (iii) (c) is performed as reported in the literature (Dubowchik and Radia, Tetrahedron Lett., 1997, 38, 5257); step (iv) (a) is performed as reported in the literature (Organic Lett., 2001, 3, 1809); step (v) is performed as reported in the literature (Corey and Venkateswarlu, J. Am. Chem. Soc., 1972, 94, 6190) and step (vi) is performed as reported by Fraser et al. (Tetrahedron Lett., 2000, 41, 1523).

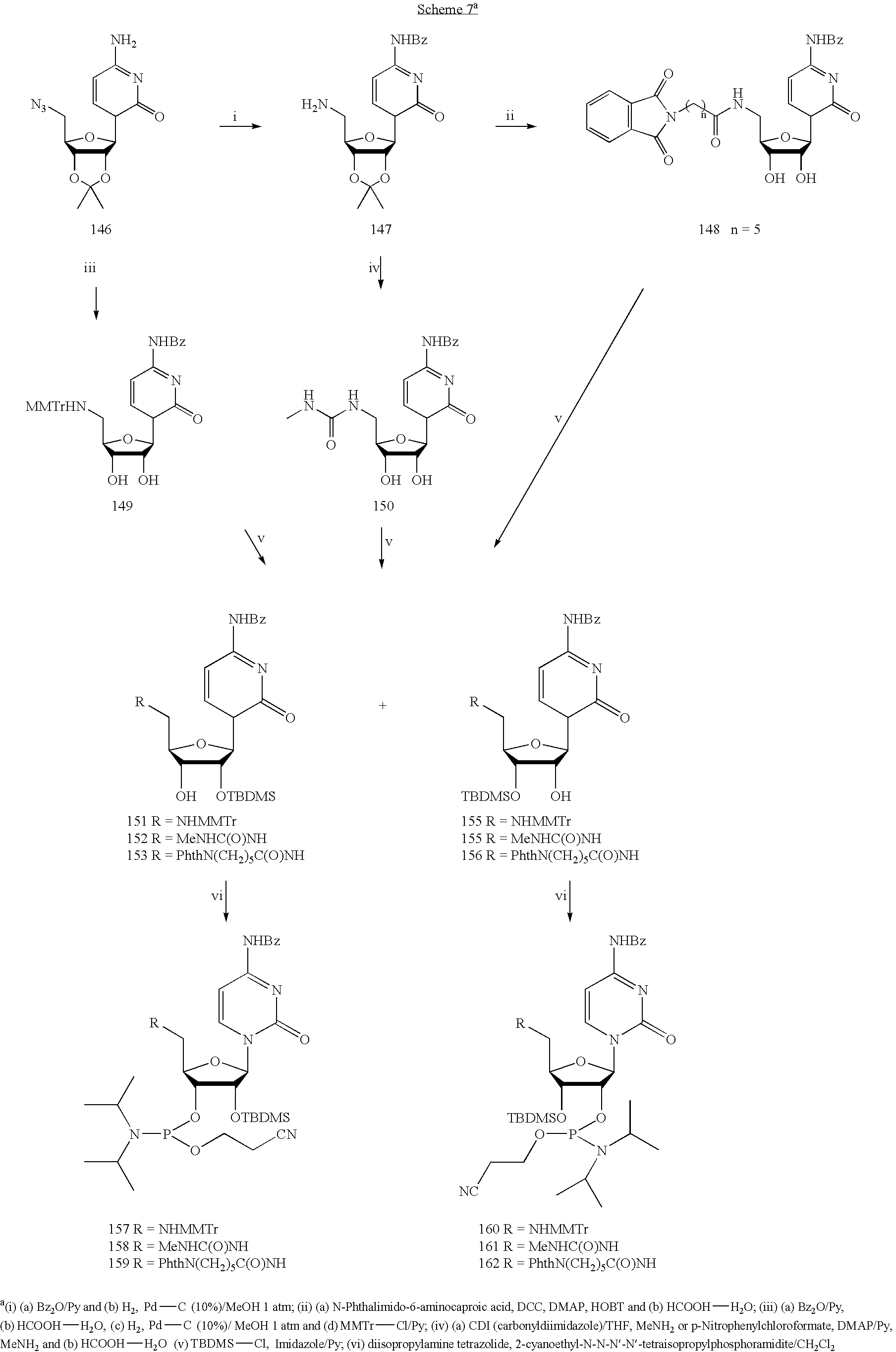

Compound 146 is obtained as reported in the literature (Liu and Austin, J. Org. Chem., 2001, 66, 8643). Step (i) (b) and (iii) (c) are performed as reported in the literature (Chem. Rev., 1954, 54, 1); step (ii) (a) is performed according to literature procedures (J. Org. Chem., 1993, 58, 2334); step (ii) (b), (iii) (b) and (iv) (b) are performed as reported in the literature (Bioorg. Med. Chem. Lett., 2003, 13, 1713); step (iii) (d) is performed as reported in the literature (Dubowchik and Radia, Tetrahedron Lett., 1997, 38, 5257); step (iv) (a) is performed as reported in the literature (Organic Lett., 2001, 3, 1809); step (v) is performed as reported in the literature (Corey and Venkateswarlu, J. Am. Chem. Soc., 1972, 94, 6190) and step (vi) is performed as reported by Fraser et al. (Tetrahedron Lett., 2000, 41, 1523)

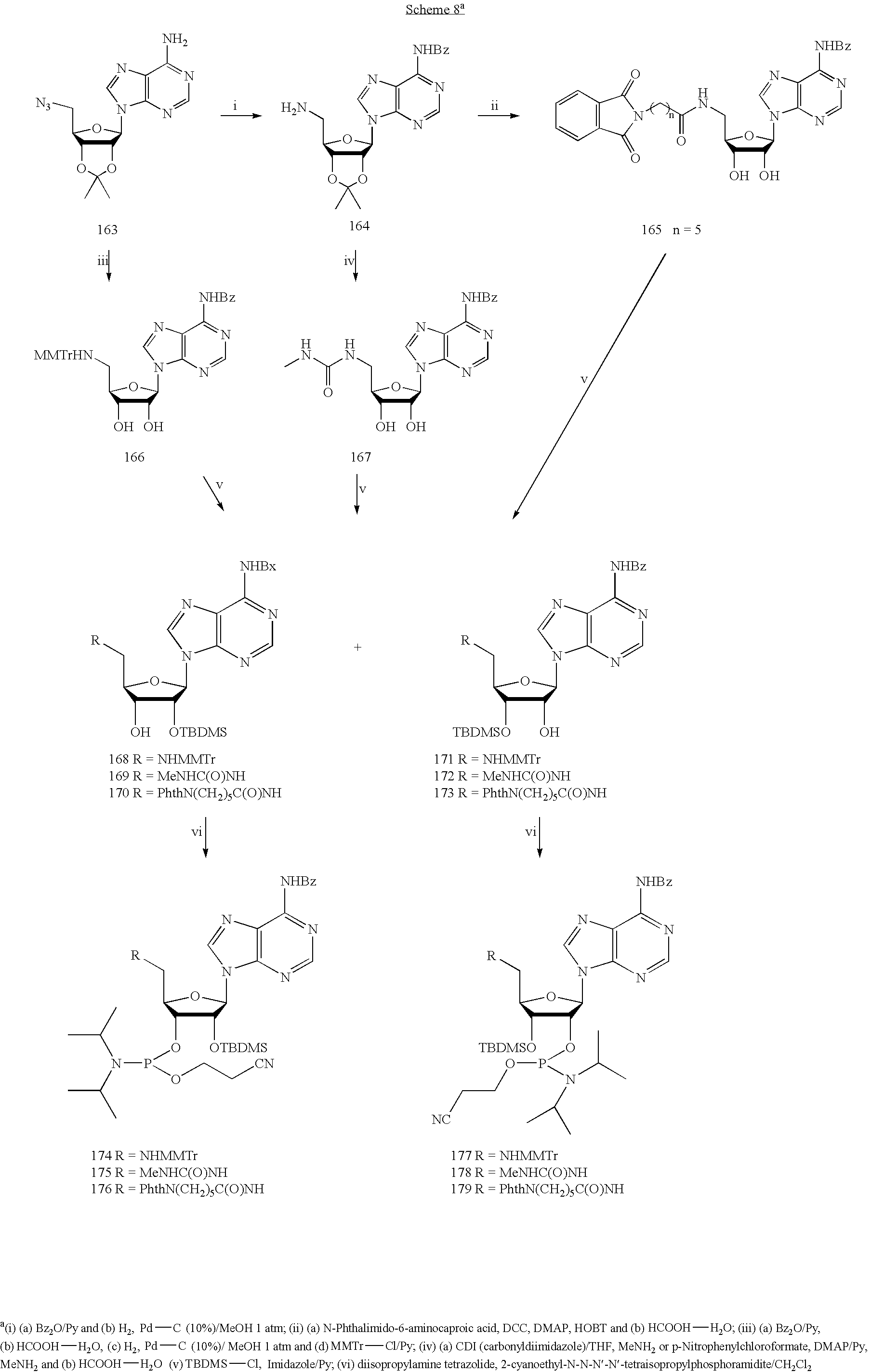

Compound 163 is obtained as reported in the literature (Liu and Austin, J. Org. Chem., 2001, 66, 8643).

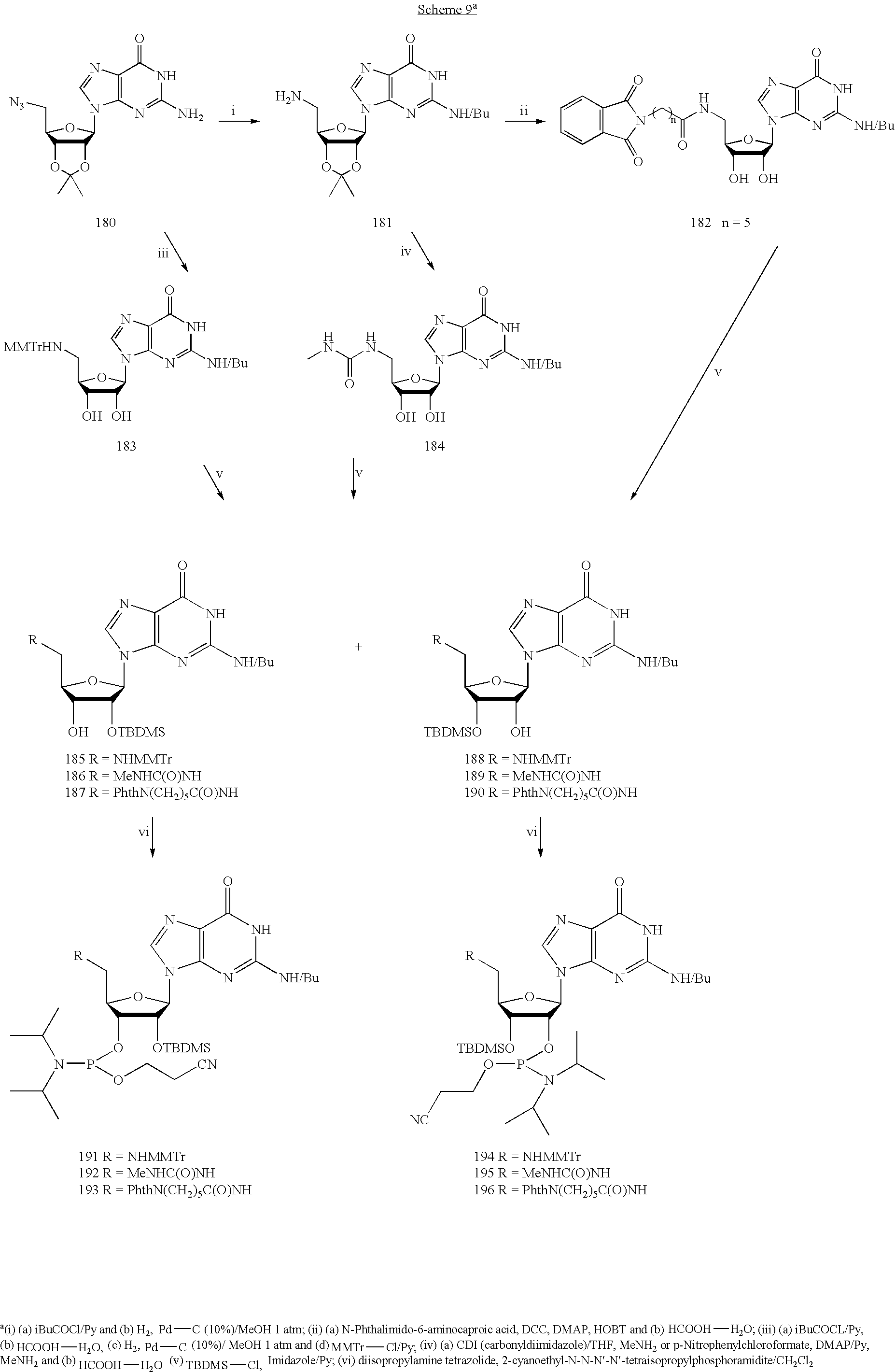

Compound 180 is obtained as reported in the literature (Liu and Austin, J. Org. Chem., 2001, 66, 8643).

Conjugation of a steroidal compound, such as choesterol, to the 3′ terminus of an antisense strand of a duplex RNA was observed to inhibit silencing. Thus placement of a steroidal compound on the 3′ terminus of the sense strand will inhibit off-target silencing. The steroidal compound can be attached to the nucleic acid strand by a linker such as a cationic linker.

Modifications to internal nucleotides (i.e., nucleotides that are not on the 5′ or 3′ terminus) were shown to inhibit silencing. Such modifications may inhibit silencing by causing the RNA sequence to resemble a DNA strand. This alteration of the sequence strand conformation may interfere with the ability of the strand to have a silencing effect. Thus, inhibition of off-target silencing can be facilitated by replacing a ribonucleotide with a deoxynucleotide. Alternatively, an internal ribonucleotide can be modified to adopt an 2′-arabino conformation, which resembles DNA in shape. For example, an internal uridine nucleotide can be replaced with an 2′-arabino-fluorodeoxyuridine.

A modification on the 5′ or 3′ terminus or on an internal nucleotide of a sense strand may have other desirable effects. For example, a modification may facilitate uptake of the iRNA agent into a cell, or may facilitate tissue-targeting. Cholesterol, for example, increases cellular uptake of iRNA agents in vitro, and can increase uptake of iRNA agents into the liver in vivo.

Any of the modifications to the 5′ or 3′ terminus or to an internal nucleotide of a sense strand may be used in combination, and may be used in combination with other modifications described herein.

In some embodiments, the off-target sequence has at least 70% complementarity to at least 10 nucleotides of the sense strand.

Design and Selection of iRNA Agents

Candidate iRNA agents can be designed by performing, for example, a gene walk analysis. Overlapping, adjacent, or closely spaced candidate agents corresponding to all or some of the transcribed region of a target gene can be generated and tested. Each of the iRNA agents can be tested and evaluated for the ability to down regulate target gene expression (see below, “Evaluation of Candidate iRNA agents”).

An iRNA agent can be rationally designed based on sequence information and desired characteristics. For example, an iRNA agent can be designed according to the relative melting temperature of the candidate duplex. Generally, the duplex will have a lower melting temperature at the 5′ end of the antisense strand than at the 3′ end of the antisense strand. This and other elements of rational design are discussed in greater detail below (see, e.g., sections labeled “Asymmetry” and “Differential Modification of Terminal Duplex Stability” and “Other-than-Watson-Crick Pairing.”

Evaluation of Candidate iRNA Agents and Candidate Sense Strand Modifications

A candidate iRNA agent can be evaluated for its ability to downregulate target gene expression and to minimize off target gene silencing. For example, a candidate iRNA agent can be provided, and contacted with a cell that expresses the target gene. The level of target gene expression prior to and following contact with the candidate iRNA agent can be compared. The target gene can be an endogenous or exogenous gene within the cell. If it is determined that the amount of RNA or protein expressed from the gene is lower following contact with the iRNA agent, then it can be concluded that the iRNA agent downregulates target gene expression. The level of target RNA or protein in the cell can be determined by any method desired. For example, the level of target RNA can be determined by Northern blot analysis, reverse transcription coupled with polymerase chain reaction (RT-PCR), or RNAse protection assay. The level of protein can be determined by Western blot analysis.

Modifications appropriate for use in inhibiting off target silencing can be evaluated. For example, a candidate modification can be applied to the 5′ or 3′ end of the antisense strand of an iRNA agent duplex having a known target RNA, or a nucleotide of the internal sequence can be modified. Levels of target RNA can be measured directly such as by Northern blot or RT-PCR, or indirectly. For example, the target RNA can encode a reporter gene, such as luciferase, or GFP, and target RNA levels can be measured by degree of reporter gene expression. A modification that is found to decrease the silencing effect of an iRNA agent can be applied to the sense strand of an iRNA agent to inhibit off-target silencing. Different modifications can be evaluated separately for unique iRNA agents, as some modifications may be more effective in combination with particular sequences, in combination with other, e.g., internal, modifications, such as those that promote nuclease resistance. Some modifications may also display differential effects on the efficacy of a therapeutic iRNA agent.

The iRNA agent can be tested in an in vitro or/and in an in vivo system. For example, the target gene or a fragment thereof can be fused to a reporter gene on a plasmid. The plasmid can be transfected into a cell with a candidate iRNA agent. The efficacy of the iRNA agent can be evaluated by monitoring expression of the reporter gene. The reporter gene can be monitored in vivo, such as by fluorescence or in situ hybridization. Exemplary fluorescent reporter genes include but are not limited to green fluorescent protein and luciferase. Expression of the reporter gene can also be monitored by Northern blot, RT-PCR, RNAse-protection assay, or Western blot analysis as described above.

Efficacy of an iRNA agent can be tested in a cell line, e.g., a mammalian cell line, such as a human cell line.

Controls include: (1) testing the efficacy and specificity of an iRNA by assaying for a decrease in expression of the target gene by, for example, comparison to expression of an endogenous or exogenous off-target RNA or protein; and (2) testing specificity of the effect on target gene expression by administering a “nonfunctional” iRNA agent.

Nonfunctional control iRNA agents can (a) target a gene not expressed in the cell; (b) be of nonsensical sequence (e.g., a scrambled version of the test iRNA); or (c) have a sequence complementary to the target gene, but be known by previous experiments to lack an ability to silence gene expression.

Assays include time course experiments to monitor stability and duration of silencing effect by an iRNA agent and monitoring in dividing versus nondividing cells. Presumably in dividing cells, the dsRNA is diluted out over time, thus decreasing the duration of the silencing effect. The implication is that dosage will have to be adjusted in vivo, and/or an iRNA agent will have to be administered more frequently to maintain the silencing effect. To monitor nondividing cells, cells can be arrested by serum withdrawal.

A candidate iRNA agent can also be evaluated for cross-species reactivity. For example, cell lines derived from different species (e.g., mouse vs. human) or in biological samples (e.g., serum or tissue extracts) isolated from different species can be transfected with a target iRNA agent and a candidate iRNA agent. The efficacy of the iRNA agent can be determined for the cell from the different species.

In Vivo Testing

An iRNA agent identified as being capable of inhibiting target gene expression can be tested for functionality in vivo in an animal model (e.g., in a mammal, such as in mouse or rat). For example, the iRNA agent can be administered to an animal, and the iRNA agent evaluated with respect to its biodistribution, stability, and its ability to inhibit target gene expression.

The iRNA agent can be administered directly to the target tissue, such as by injection, or the iRNA agent can be administered to the animal model in the same manner that it would be administered to a human. For example, the iRNA agent can be injected directly into a target region of the brain (e.g., into the cortex, the substantia nigra, the globus pallidus, or the hippocampus), and after a period of time, the brain can be harvested and tissue slices examined for distribution of the agent.

The iRNA agent can also be evaluated for its intracellular distribution. The evaluation can include determining whether the iRNA agent was taken up into the cell. The evaluation can also include determining the stability (e.g., the half-life) of the iRNA agent. Evaluation of an iRNA agent in vivo can be facilitated by use of an iRNA agent conjugated to a traceable marker (e.g., a fluorescent marker such as fluorescein; a radioactive label, such as ³²P, ³³P, or ³H; gold particles; or antigen particles for immunohistochemistry).

An iRNA agent useful for monitoring biodistribution can lack gene silencing activity in vivo. For example, the iRNA agent can target a gene not present in the animal (e.g., an iRNA agent injected into mouse can target luciferase), or an iRNA agent can have a non-sense sequence, which does not target any gene, e.g., any endogenous gene). Localization/biodistribution of the iRNA can be monitored by a traceable label attached to the iRNA agent, such as a traceable agent described above

The iRNA agent can be evaluated with respect to its ability to down regulate target gene expression. Levels of target gene expression in vivo can be measured, for example, by in situ hybridization, or by the isolation of RNA from tissue prior to and following exposure to the iRNA agent. target RNA can be detected by any desired method, including but not limited to RT-PCR, Northern blot, or RNAase protection assay. Alternatively, or additionally, target gene expression can be monitored by performing Western blot analysis on tissue extracts treated with the iRNA agent.

iRNA Chemistry

Described herein are isolated iRNA agents, e.g., RNA molecules, (double-stranded; single-stranded) that mediate RNAi. The iRNA agents preferably mediate RNAi with respect to an endogenous target gene gene of a subject

The iRNA agent should include a region of sufficient homology to the target gene, and be of sufficient length in terms of nucleotides, such that the iRNA agent, or a fragment thereof, can mediate down regulation of the target gene. (For ease of exposition the term nucleotide or ribonucleotide is sometimes used herein in reference to one or more monomeric subunits of an RNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA.

Complementarity, or degree of homology with the target strand, is most critical in the antisense strand. While perfect complementarity, particularly in the antisense strand, is often desired some embodiments can include, particularly in the antisense strand, one or more but preferably 6, 5, 4, 3, 2, or fewer mismatches (with respect to the target RNA). The mismatches, particularly in the antisense strand, are most tolerated in the terminal regions and if present are preferably in a terminal region or regions, e.g., within 6, 5, 4, or 3 nucleotides of the 5′ and/or 3′ terminus. The sense strand need only be sufficiently complementary with the antisense strand to maintain the over all double strand character of the molecule.

Single stranded regions of an iRNA agent will often be modified or include nucleoside surrogates, e.g., the unpaired region or regions of a hairpin structure, e.g., a region which links two complementary regions, can have modifications or nucleoside surrogates. Modifications to stabilize one or both of the 3′- or 5′-terminus of an iRNA agent, e.g., against exonucleases, or to favor the antisense sRNA agent to enter into RISC are also favored. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis. As discussed elsewhere herein, an iRNA agent will often be modified or include a ribose replacement monomer subunit (RRMS) in addition to the nucleotide surrogate. An RRMS replaces a ribose sugar on a ribonucleotide with another moiety, e.g., a non-carbohydrate (preferably cyclic) carrier. RRMS′ are described in greater detail below.

iRNA agents include molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed sRNA agents or shorter iRNA agents herein. “sRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60 but preferably less than 50, 40, or 30 nucleotide pairs. The sRNA agent, or a cleavage product thereof, can down regulate a target gene, e.g., by inducing RNAi with respect to a target RNA, preferably an endogenous or pathogen target RNA.

Each strand of an sRNA agent can be equal to or less than 30, 25, 24, 23, 22, 21, or 20 nucleotides in length. The strand is preferably at least 19 nucleotides in length. For example, each strand can be between 21 and 25 nucleotides in length. Preferred sRNA agents have a duplex region of 17, 18, 19, 29, 21, 22, 23, 24, or 25 nucleotide pairs, and one or more overhangs, preferably one or two 3′ overhangs, of 2-3 nucleotides.

In addition to homology to target RNA and the ability to down regulate a target gene, an iRNA agent will preferably have one or more of the following properties:
(1) it will be of the Formula 1, 2, 3, or 4 set out in the RNA Agent section below;
(2) if single stranded it will have a 5′ modification which includes one or more phosphate groups or one or more analogs of a phosphate group;
(3) it will, despite modifications, even to a very large number, or all of the nucleosides, have an antisense strand that can present bases (or modified bases) in the proper three dimensional framework so as to be able to form correct base pairing and form a duplex structure with a homologous target RNA which is sufficient to allow down regulation of the target, e.g., by cleavage of the target RNA;
(4) it will, despite modifications, even to a very large number, or all of the nucleosides, still have “RNA-like” properties, i.e., it will possess the overall structural, chemical and physical properties of an RNA molecule, even though not exclusively, or even partly, of ribonucleotide-based content. For example, an iRNA agent can contain, e.g., a sense and/or an antisense strand in which all of the nucleotide sugars contain e.g., 2′ fluoro in place of 2′ hydroxyl. This deoxyribonucleotide-containing agent can still be expected to exhibit RNA-like properties. While not wishing to be bound by theory, the electronegative fluorine prefers an axial orientation when attached to the C2′ position of ribose. This spatial preference of fluorine can, in turn, force the sugars to adopt a C_{3′}-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. (Generally, it is preferred that a modified moiety at the 2′ sugar position will be able to enter into H-bonding which is more characteristic of the OH moiety of a ribonucleotide than the H moiety of a deoxyribonucleotide. A preferred iRNA agent will: exhibit a C_{3′}-endo pucker in all, or at least 50, 75, 80, 85, 90, or 95% of its sugars; exhibit a C_{3′}-endo pucker in a sufficient amount of its sugars that it can give rise to a the RNA-characteristic A-family-type helix; will have no more than 20, 10, 5, 4, 3, 2, or 1 sugar which is not a C_{3′}-endo pucker structure. These limitations are particularly preferably in the antisense strand;
(5) regardless of the nature of the modification, and even though the RNA agent can contain deoxynucleotides or modified deoxynucleotides, particularly in overhang or other single strand regions, it is preferred that DNA molecules, or any molecule in which more than 50, 60, or 70% of the nucleotides in the molecule, or more than 50, 60, or 70% of the nucleotides in a duplexed region are deoxyribonucleotides, or modified deoxyribonucleotides which are deoxy at the 2′ position, are excluded from the definition of RNA agent.

A “single strand iRNA agent” as used herein, is an iRNA agent which is made up of a single molecule. It may include a duplexed region, formed by intra-strand pairing, e.g., it may be, or include, a hairpin or panhandle structure. Single strand iRNA agents are preferably antisense with regard to the target molecule. In preferred embodiments single strand iRNA agents are 5′ phosphorylated or include a phosphoryl analog at the 5′ prime terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2—), 5′-alkyletherphosphonates (R=alkylether=methoxymethyl (MeOCH2—), ethoxymethyl, etc., e.g. RP(OH)(O)—O-5′-). (These modifications can also be used with the antisense strand of a double stranded iRNA.)

A single strand iRNA agent should be sufficiently long that it can enter the RISC and participate in RISC mediated cleavage of a target mRNA. A single strand iRNA agent is at least 14, and more preferably at least 15, 20, 25, 29, 35, 40, or 50 nucleotides in length. It is preferably less than 200, 100, or 60 nucleotides in length.

Hairpin iRNA agents will have a duplex region equal to or at least 17, 18, 19, 29, 21, 22, 23, 24, or 25 nucleotide pairs. The duplex region will preferably be equal to or less than 200, 100, or 50, in length. Preferred ranges for the duplex region are 15-30, 17 to 23, 19 to 23, and 19 to 21 nucleotides pairs in length. The hairpin will preferably have a single strand overhang or terminal unpaired region, preferably the 3′, and preferably of the antisense side of the hairpin. Preferred overhangs are 2-3 nucleotides in length.

A “double stranded (ds) iRNA agent” as used herein, is an iRNA agent which includes more than one, and preferably two, strands in which interchain hybridization can form a region of duplex structure.

The antisense strand of a double stranded iRNA agent should be equal to or at least, 14, 15, 16 17, 18, 19, 25, 29, 40, or 60 nucleotides in length. It should be equal to or less than 200, 100, or 50, nucleotides in length. Preferred ranges are 17 to 25, 19 to 23, and 19 to 21 nucleotides in length.

The sense strand of a double stranded iRNA agent should be equal to or at least 14, 15, 16 17, 18, 19, 25, 29, 40, or 60 nucleotides in length. It should be equal to or less than 200, 100, or 50, nucleotides in length. Preferred ranges are 17 to 25, 19 to 23, and 19 to 21 nucleotides in length.

The double strand portion of a double stranded iRNA agent should be equal to or at least, 14, 15, 16 17, 18, 19, 20, 21, 22, 23, 24, 25, 29, 40, or 60 nucleotide pairs in length. It should be equal to or less than 200, 100, or 50, nucleotides pairs in length. Preferred ranges are 15-30, 17 to 23, 19 to 23, and 19 to 21 nucleotides pairs in length.

In many embodiments, the ds iRNA agent is sufficiently large that it can be cleaved by an endogenous molecule, e.g., by Dicer, to produce smaller ds iRNA agents, e.g., sRNAs agents

It may be desirable to modify one or both of the antisense and sense strands of a double strand iRNA agent. In some cases they will have the same modification or the same class of modification but in other cases the sense and antisense strand will have different modifications, e.g., in some cases it is desirable to modify only the sense strand. It may be desirable to modify only the sense strand, e.g., to inactivate it, e.g., the sense strand can be modified in order to inactivate the sense strand and prevent formation of an active sRNA/protein or RISC. This can be accomplished by a modification which prevents 5′-phosphorylation of the sense strand, e.g., by modification with a 5′-O-methyl ribonucleotide (see Nykänen et al., (2001) ATP requirements and small interfering RNA structure in the RNA interference pathway. Cell 107, 309-321.) Other modifications which prevent phosphorylation can also be used, e.g., simply substituting the 5′-OH by H rather than O-Me. Alternatively, a large bulky group may be added to the 5′-phosphate turning it into a phosphodiester linkage, though this may be less desirable as phosphodiesterases can cleave such a linkage and release a functional sRNA 5′-end. Antisense strand modifications include 5′ phosphorylation as well as any of the other 5′ modifications discussed herein, particularly the 5′ modifications discussed above in the section on single stranded iRNA molecules.

It is preferred that the sense and antisense strands be chosen such that the ds iRNA agent includes a single strand or unpaired region at one or both ends of the molecule. Thus, a ds iRNA agent contains sense and antisense strands, preferably paired to contain an overhang, e.g., one or two 5′ or 3′ overhangs but preferably a 3′ overhang of 2-3 nucleotides. Most embodiments will have a 3′ overhang. Preferred sRNA agents will have single-stranded overhangs, preferably 3′ overhangs, of 1 or preferably 2 or 3 nucleotides in length at each end. The overhangs can be the result of one strand being longer than the other, or the result of two strands of the same length being staggered. 5′ ends are preferably phosphorylated.

Preferred lengths for the duplexed region is between 15 and 30, most preferably 18, 19, 20, 21, 22, and 23 nucleotides in length, e.g., in the sRNA agent range discussed above. sRNA agents can resemble in length and structure the natural Dicer processed products from long dsRNAs. Embodiments in which the two strands of the sRNA agent are linked, e.g., covalently linked are also included. Hairpin, or other single strand structures which provide the required double stranded region, and preferably a 3′ overhang are also within the invention.

The isolated iRNA agents described herein, including ds iRNA agents and sRNA agents can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, e.g., the target gene gene.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an sRNA agent of 21 to 23 nucleotides.

As used herein, “specifically hybridizable” and “complementary” are terms which are used to indicate a sufficient degree of complementarity such that stable and specific binding occurs between a compound of the invention and a target RNA molecule. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In one embodiment, an iRNA agent is “sufficiently complementary” to a target RNA, e.g., a target mRNA (e.g., a target SCNA mRNA) such that the iRNA agent silences production of a protein encoded by the target mRNA. In another embodiment, the iRNA agent is “exactly complementary” (excluding the RRMS containing subunit(s) to the target RNA, e.g., the target RNA and the iRNA agent anneal, preferably to form a hybrid made exclusively of Watson-Crick basepairs in the region of exact complementarity. A “sufficiently complementary” target RNA can include an internal region (e.g., of at least 10 nucleotides) that is exactly complementary to a target RNA. Moreover, in some embodiments, the iRNA agent specifically discriminates a single-nucleotide difference. In this case, the iRNA agent only mediates RNAi if exact complementary is found in the region (e.g., within 7 nucleotides of) the single-nucleotide difference.

As used herein, the term “oligonucleotide” refers to a nucleic acid molecule (RNA or DNA) preferably of length less than 100, 200, 300, or 400 nucleotides.

RNA agents discussed herein include otherwise unmodified RNA as well as RNA which have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al., (1994) Nucleic Acids Res. 22: 2183-2196. Such rare or unusual RNAs, often termed modified RNAs (apparently because the are typically the result of a post transcriptionally modification) are within the term unmodified RNA, as used herein. Modified RNA as used herein refers to a molecule in which one or more of the components of the nucleic acid, namely sugars, bases, and phosphate moieties, are different from that which occur in nature, preferably different from that which occurs in the human body. While they are referred to as modified “RNAs,” they will of course, because of the modification, include molecules which are not RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone. Examples of all of the above are discussed herein.

Much of the discussion below refers to single strand molecules. In many embodiments of the invention a double stranded iRNA agent, e.g., a partially double stranded iRNA agent, is required or preferred. Thus, it is understood that that double stranded structures (e.g. where two separate molecules are contacted to form the double stranded region or where the double stranded region is formed by intramolecular pairing (e.g., a hairpin structure)) made of the single stranded structures described below are within the invention. Preferred lengths are described elsewhere herein.

As nucleic acids are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid but in many, and in fact in most, cases it will not. By way of example, a modification may only occur at a 3′ or 5′ terminal position, may only occur in a terminal region, e.g. at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. The 5′ end or ends can be phosphorylated.

In some embodiments it is particularly preferred, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

Modifications and nucleotide surrogates are discussed below.

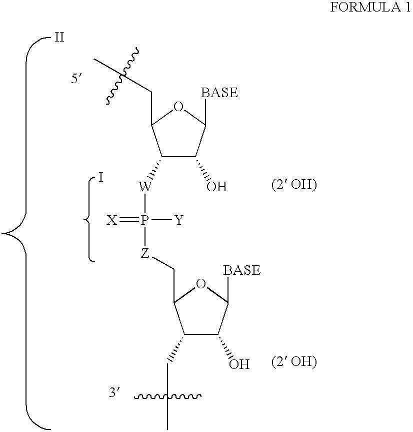

The scaffold presented above in Formula 1 represents a portion of a ribonucleic acid. The basic components are the ribose sugar, the base, the terminal phosphates, and phosphate internucleotide linkers. Where the bases are naturally occurring bases, e.g., adenine, uracil, guanine or cytosine, the sugars are the unmodified 2′ hydroxyl ribose sugar (as depicted) and W, X, Y, and Z are all O, Formula 1 represents a naturally occurring unmodified oligoribonucleotide.

Unmodified oligoribonucleotides may be less than optimal in some applications, e.g., unmodified oligoribonucleotides can be prone to degradation by e.g., cellular nucleases. Nucleases can hydrolyze nucleic acid phosphodiester bonds. However, chemical modifications to one or more of the above RNA components can confer improved properties, and, e.g., can render oligoribonucleotides more stable to nucleases. Unmodified oligoribonucleotides may also be less than optimal in terms of offering tethering points for attaching ligands or other moieties to an iRNA agent.

Modified nucleic acids and nucleotide surrogates can include one or more of:
(i) alteration, e.g., replacement, of one or both of the non-linking (X and Y)phosphate oxygens and/or of one or more of the linking (W and Z)phosphate oxygens (When the phosphate is in the terminal position, one of the positions W or Z will not link the phosphate to an additional element in a naturally occurring ribonucleic acid. However, for simplicity of terminology, except where otherwise noted, the W position at the 5′ end of a nucleic acid and the terminal Z position at the 3′ end of a nucleic acid, are within the term “linking phosphate oxygens” as used herein.);
(ii) alteration, e.g., replacement, of a constituent of the ribose sugar, e.g., of the 2′ hydroxyl on the ribose sugar, or wholesale replacement of the ribose sugar with a structure other than ribose, e.g., as described herein;
(iii) wholesale replacement of the phosphate moiety (bracket I) with “dephospho” linkers;
(iv) modification or replacement of a naturally occurring base;
(v) replacement or modification of the ribose-phosphate backbone (bracket II);
(vi) modification of the 3′ end or 5′ end of the RNA, e.g., removal, modification or replacement of a terminal phosphate group or conjugation of a moiety, e.g. a fluorescently labeled moiety, to either the 3′ or 5′ end of RNA.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species. For example, any phosphoroamidate (replacement of a nonlinking oxygen with nitrogen) would be represented by X═O and Y═N in the above figure.

The Phosphate Group

The phosphate group is a negatively charged species. The charge is distributed equally over the two non-linking oxygen atoms (i.e., X and Y in Formula 1 above). However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. Unlike the situation where only one of X or Y is altered, the phosphorus center in the phosphorodithioates is achiral which precludes the formation of oligoribonucleotides diastereomers. Diastereomer formation can result in a preparation in which the individual diastereomers exhibit varying resistance to nucleases. Further, the hybridization affinity of RNA containing chiral phosphate groups can be lower relative to the corresponding unmodified RNA species. Thus, while not wishing to be bound by theory, modifications to both X and Y which eliminate the chiral center, e.g. phosphorodithioate formation, may be desirable in that they cannot produce diastereomer mixtures. Thus, X can be any one of S, Se, B, C, H, N, or OR (R is alkyl or aryl). Thus Y can be any one of S, Se, B, C, H, N, or OR (R is alkyl or aryl). Replacement of X and/or Y with sulfur is preferred.

The phosphate linker can also be modified by replacement of a linking oxygen (i.e., W or Z in Formula 1) with nitrogen (bridged phosphoroamidates), sulfur (bridged phosphorothioates) and carbon (bridged methylenephosphonates). The replacement can occur at a terminal oxygen (position W (3′) or position Z (5′). Replacement of W with carbon or Z with nitrogen is preferred.

Candidate agents can be evaluated for suitability as described below.

The Sugar Group

A modified RNA can include modification of all or some of the sugar groups of the ribonucleic acid. E.g., the 2′ or 3′ hydroxyl group (OH) can be modified or replaced with a number of different “oxy” or “deoxy” substituents. While not being bound by theory, enhanced stability is expected since the hydroxyl can no longer be deprotonated to form a 2′ alkoxide ion. The 2′ alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ or 3′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Preferred substitutents are 2′-methoxyethyl, 2′-OCH3, 2′—O-allyl, 2′-C-allyl, and 2′-fluoro (and the corresponding 3′ substituents)

The sugar group can also contain one or more carbons that possess the opposite stereochemical configuration than that of the corresponding carbon in ribose. Thus, a modified RNA can include nucleotides containing e.g., arabinose, as the sugar.

Modified RNAs can also include “abasic” sugars, which lack a nucleobase at C-1′. These abasic sugars can also be further contain modifications at one or more of the constituent sugar atoms.

To maximize nuclease resistance, the 2′ or 3′ modifications can be used in combination with one or more phosphate linker modifications (e.g., phosphorothioate). The so-called “chimeric” oligonucleotides are those that contain two or more different modifications.

The modificaton can also entail the wholesale replacement of a ribose structure with another entity at one or more sites in the iRNA agent. These modifications are described in section entitled Ribose Replacements for RRMSs.

Candidate modifications can be evaluated as described below.

Replacement of the Phosphate Group

The phosphate group can be replaced by non-phosphorus containing connectors (cf. Bracket I in Formula 1 above). While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

Candidate modifications can be evaluated as described below.

Replacement of Ribophosphate Backbone

Oligonucleotide-mimicking scaffolds can also be constructed wherein the phosphate linker and ribose sugar are replaced by nuclease resistant nucleoside or nucleotide surrogates (see Bracket II of Formula 1 above). While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone.

Examples include the mophilino, cyclobutyl, pyrrolidine and peptide nucleic acid (PNA) nucleoside surrogates. A preferred surrogate is a PNA surrogate.

Candidate modifications can be evaluated as described below.

Terminal Modifications

The 3′ and 5′ ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a spacer. The terminal atom of the spacer can connect to or replace the linking atom of the phosphate group or the C-3′ or C-5′ O, N, S or C group of the sugar. Alternatively, the spacer can connect to or replace the terminal atom of a nucleotide surrogate (e.g., PNAs). These spacers or linkers can include e.g., —(CH₂)ₙ—, —(CH₂)ₙN—, —(CH₂)ₙO—, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents. When a spacer/phosphate-functional molecular entity-spacer/phosphate array is interposed between two strands of iRNA agents, this array can substitute for a hairpin RNA loop in a hairpin-type RNA agent. The 3′ end can be an —OH group. While not wishing to be bound by theory, it is believed that conjugation of certain moieties can improve transport, hybridization, and specificity properties. Again, while not wishing to be bound by theory, it may be desirable to introduce terminal alterations that improve nuclease resistance. Other examples of terminal modifications include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can be added for a number of reasons, including as discussed elsewhere herein to modulate activity or to modulate resistance to degradation. Terminal modifications useful for modulating activity include modification of the 5′ end with phosphate or phosphate analogs. E.g., in preferred embodiments iRNA agents, especially antisense strands, are 5′ phosphorylated or include a phosphoryl analog at the 5′ prime terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxgen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl(MeOCH2-), ethoxymethyl, etc., e.g. RP(OH)(O)—O-5′-).

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include cholesterol. Terminal modifications can also be useful for cross-linking an RNA agent to another moiety; modifications useful for this include mitomycin C.

Evaluation of iRNA Agents

One can evaluate a candidate iRNA agent, e.g., a modified iRNA agent. In general, one can test for a selected property by exposing the agent or modified molecule and a control molecule to the appropriate conditions and evaluating for the presence of the selected property. For example, resistance to a degradent can be evaluated as follows. A candidate modified RNA (and preferably a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled, preferably prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified siRNA homologous to the GFP mRNA can be assayed for the ability to inhibit GFP expression by monitoring for a decrease in cell fluorescence, as compared to a control cell, in which the transfection did not include the candidate siRNA, e.g., controls with no agent added and/or controls with a non-modified RNA added. Efficacy of the candidate agent on gene expression can be assessed by comparing cell fluorescence in the presence of the modified and unmodified iRNA agents.

The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

Preferred iRNA Agents

Preferred RNA agents have the following structure (see Formula 2 below):

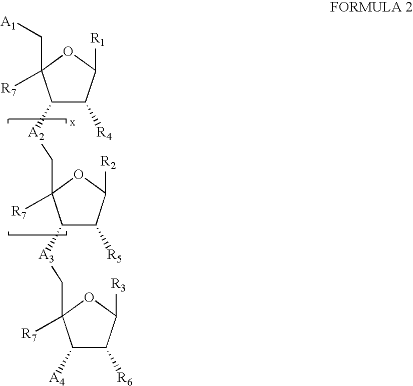

Referring to Formula 2 above, R¹, R², and R³ are each, independently, H, (i.e. abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6,N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N-6-isopentenyladenine, N-methylguanines, or O-alkylated bases.

R⁴, R⁵, and R⁶ are each, independently, OR⁸, O(CH₂CH₂O)ₘCH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂NHR⁹; NHC(O)R⁸; cyano; mercapto, SR⁸; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, or ureido; or R⁴, R⁵, or R⁶ together combine with R⁷ to form an [—O—CH₂—] covalently bound bridge between the sugar 2′ and 4′ carbons.

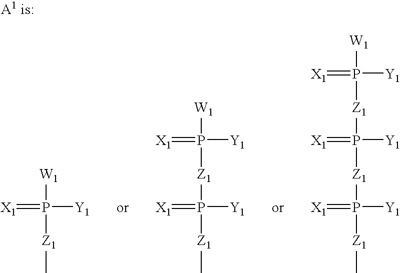

; H; OH; OCH₃; W¹; an abasic nucleotide; or absent;

(a preferred A1, especially with regard to anti-sense strands, is chosen from 5′-monophosphate ((HO)₂(O)P—O-5′), 5′-diphosphate ((HO)₂(O)P—O—P(HO)(O)—O-5′), 5′-triphosphate ((HO)₂(O)P—O—(HO)(O)P—O—P(HO)(O)—O-5′), 5′-guanosine cap (7-methylated or non-methylated) (7m-G-O-5′-(HO)(O)P—O—(HO)(O)P—O—P(HO)(O)—O-5′), 5′-adenosine cap (Appp), and any modified or unmodified nucleotide cap structure (N—O-5′-(HO)(O)P—O—(HO)(O)P—O—P(HO)(O)—O-5′), 5′-monothiophosphate (phosphorothioate; (HO)₂(S)P—O-5′), 5′-monodithiophosphate (phosphorodithioate; (HO)(HS)(S)P—O-5′), 5′-phosphorothiolate ((HO)₂(O)P—S-5′); any additional combination of oxgen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, (OH)₂(O)P-5′-CH₂—), 5′-alkyletherphosphonates (R=alkylether=methoxymethyl (MeOCH₂—), ethoxymethyl, etc., e.g. RP(OH)(O)—O-5′-)).

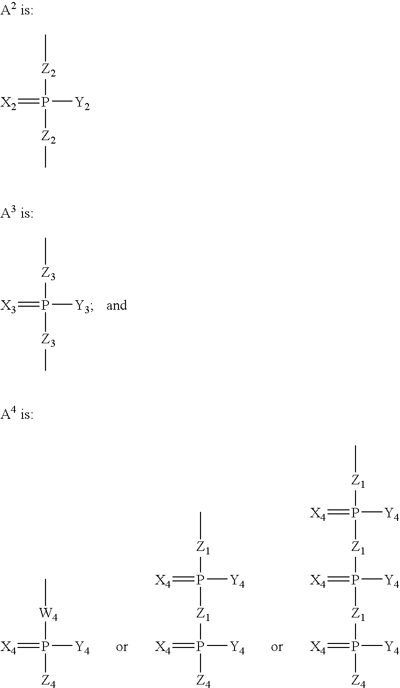

;H; Z⁴; an inverted nucleotide; an abasic nucleotide; or absent.

W¹ is OH, (CH₂)ₙR¹⁰, (CH₂)ₙNHR¹⁰, (CH₂)ₙ OR¹⁰, (CH₂)ₙ SR¹⁰; O(CH₂)ₙR¹⁰; O(CH₂)ₙOR¹⁰, O(CH₂)ₙNR¹⁰, O(CH₂)ₙSR¹⁰; O(CH₂)ₙSS(CH₂)ₙOR¹⁰, O(CH₂)ₙC(O)OR¹⁰, NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰ O(CH₂CH₂O)ₘCH₂CH₂OR¹⁰; O(CH₂CH₂O)ₘCH₂CH₂NHR¹⁰, NH(CH₂CH₂NH)ₘCH₂CH₂NHR¹⁰; Q-R¹⁰, O-Q-R¹⁰ N-Q-R¹⁰, S-Q-R¹⁰ or —O—, W⁴ is O, CH₂, NH, or S.

X¹, X², X³, and X⁴ are each, independently, O or S.

Y¹, Y², Y³, and Y⁴ are each, independently, OH, O⁻, OR⁸, S, Se, BH₃⁻, H, NHR⁹, N(R⁹)₂ alkyl, cycloalkyl, aralkyl, aryl, or heteroaryl, each of which may be optionally substituted.

Z¹, Z², and Z³ are each independently O, CH₂, NH, or S. Z⁴ is OH, (CH₂)ₙR¹⁰, (CH₂)ₙNHR¹⁰, (CH₂)ₙ OR¹⁰, (CH₂)ₙ SR¹⁰; O(CH₂)ₙR¹⁰; O(CH₂)ₙOR¹⁰, O(CH₂)ₙNR¹⁰, O(CH₂)ₙSR¹⁰, O(CH₂)ₙSS(CH₂)ₙOR¹⁰, O(CH₂)ₙC(O)OR¹⁰; NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰O(CH₂CH₂O)ₘCH₂CH₂OR¹⁰, O(CH₂CH₂O)ₘCH₂CH₂NHR¹⁰, NH(CH₂CH₂NH)ₘCH₂CH₂NHR¹⁰; Q-R¹⁰, O-Q-R¹⁰N-Q-R¹⁰, S-Q-R¹⁰.

x is 5-100, chosen to comply with a length for an RNA agent described herein.

R⁷ is H; or is together combined with R⁴, R⁵, or R⁶ to form an [—O—CH₂—] covalently bound bridge between the sugar 2′ and 4′ carbons.

R⁸ is alkyl, cycloalkyl, aryl, aralkyl, heterocyclyl, heteroaryl, amino acid, or sugar; R⁹ is NH₂, alkylamino, dialkylamino, heterocyclyl, arylamino, diaryl amino, heteroaryl amino, diheteroaryl amino, or amino acid; and R¹⁰ is H; fluorophore (pyrene, TAMRA, fluorescein, Cy3 or Cy5 dyes); sulfur, silicon, boron or ester protecting group; intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]₂, polyamino; alkyl, cycloalkyl, aryl, aralkyl, heteroaryl; radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles); or an RNA agent. m is 0-1,000,000, and n is 0-20. Q is a spacer selected from the group consisting of abasic sugar, amide, carboxy, oxyamine, oxyimine, thioether, disulfide, thiourea, sulfonamide, or morpholino, biotin or fluorescein reagents.

Preferred RNA agents in which the entire phosphate group has been replaced have the following structure (see Formula 3 below):

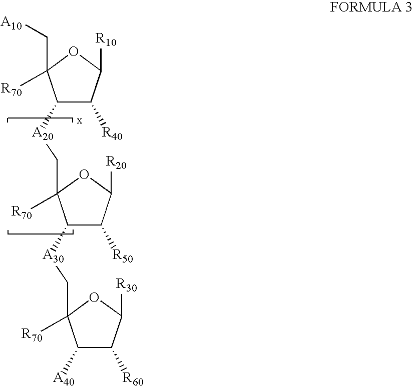

Referring to Formula 3, A¹⁰-A⁴⁰ is L-G-L; A¹⁰ and/or A⁴⁰ may be absent, in which L is a linker, wherein one or both L may be present or absent and is selected from the group consisting of CH₂(CH₂)_{g}; N(CH₂)_{g}; O(CH₂)_{g}; S(CH₂)_{g}. G is a functional group selected from the group consisting of siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

R¹⁰, R²⁰, and R³⁰ are each, independently, H, (i.e. abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6,N6-dimethyladenine, 2,6-diaminopurine, 5-amino-allyl-uracil, N3-methyluracil substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N-6-isopentenyladenine, N-methylguanines, or O-alkylated bases.

R⁴⁰, R⁵⁰, and R⁶⁰ are each, independently, OR⁸, O(CH₂CH₂O)ₘCH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂R⁹; NHC(O)R⁸; cyano; mercapto, SR⁷; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, and ureido groups; or R⁴⁰, R⁵⁰, or R⁶⁰ together combine with R⁷⁰ to form an [—O—CH₂—] covalently bound bridge between the sugar 2′ and 4′ carbons.

x is 5-100 or chosen to comply with a length for an RNA agent described herein.

R⁷⁰ is H; or is together combined with R⁴⁰, R⁵⁰, or R⁶⁰ to form an [—O—CH₂—] covalently bound bridge between the sugar 2′ and 4′ carbons.

R⁸ is alkyl, cycloalkyl, aryl, aralkyl, heterocyclyl, heteroaryl, amino acid, or sugar; and R⁹ is NH₂, alkylamino, dialkylamino, heterocyclyl, arylamino, diaryl amino, heteroaryl amino, diheteroaryl amino, or amino acid. m is 0-1,000,000, n is 0-20, and g is 0-2.

Preferred nucleoside surrogates have the following structure (see Formula 4 below):
SLR¹⁰⁰-(M-SLR²⁰⁰)ₓ-M-SLR³⁰⁰

### Formula 4

S is a nucleoside surrogate selected from the group consisting of mophilino, cyclobutyl, pyrrolidine and peptide nucleic acid. L is a linker and is selected from the group consisting of CH₂(CH₂)_{g}; N(CH₂)_{g}; O(CH₂)_{g}; S(CH₂)_{g}; —C(O)(CH₂)ₙor may be absent. M is an amide bond; sulfonamide; sulfinate; phosphate group; modified phosphate group as described herein; or may be absent.

R¹⁰⁰, R²⁰⁰, and R³⁰⁰ are each, independently, H (i.e., abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6,N6-dimethyladenine, 2,6-diaminopurine, 5-amino-allyl-uracil, N3-methyluracil substituted 1,2,4,-triazoles, 2-pyridinones, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N-6-isopentenyladenine, N-methylguanines, or O-alkylated bases.

x is 5-100, or chosen to comply with a length for an RNA agent described herein; and g is 0-2.

Nuclease Resistant Monomers

An RNA, e.g., an iRNA agent, can incorporate a nuclease resistant monomer (NRM). For example, the invention includes an iRNA agent described herein, e.g., an iRNA agent having a modification of the sense strand to inhibit off-site targeting, an iRNA agent having a non canonical pairing, an iRNA agent having an architecture or structure described herein, an iRNA associated with an amphipathic delivery agent described herein, an iRNA associated with a drug delivery module described herein, an iRNA agent administered as described herein, or an iRNA agent formulated as described herein, which also incorporates an NRM.

An iRNA agent can include monomers which have been modified so as to inhibit degradation, e.g., by nucleases, e.g., endonucleases or exonucleases, found in the body of a subject. These monomers are referred to herein as NRMs, or nuclease resistance promoting monomers or modifications. In many cases these modifications will modulate other properties of the iRNA agent as well, e.g., the ability to interact with a protein, e.g., a transport protein, e.g., serum albumin, or a member of the RISC (RNA-induced Silencing Complex), or the ability of the first and second sequences to form a duplex with one another or to form a duplex with another sequence, e.g., a target molecule.

While not wishing to be bound by theory, it is believed that modifications of the sugar, base, and/or phosphate backbone in an iRNA agent can enhance endonuclease and exonuclease resistance, and can enhance interactions with transporter proteins and one or more of the functional components of the RISC complex. Preferred modifications are those that increase exonuclease and endonuclease resistance and thus prolong the half-life of the iRNA agent prior to interaction with the RISC complex, but at the same time do not render the iRNA agent resistant to endonuclease activity in the RISC complex. Again, while not wishing to be bound by any theory, it is believed that placement of the modifications at or near the 3′ and/or 5′ end of antisense strands can result in iRNA agents that meet the preferred nuclease resistance criteria delineated above. Again, still while not wishing to be bound by any theory, it is believed that placement of the modifications at e.g., the middle of a sense strand can result in iRNA agents that are relatively less likely to undergo off-targeting.

Modifications described herein can be incorporated into any double-stranded RNA and RNA-like molecule described herein, e.g., an iRNA agent. An iRNA agent may include a duplex comprising a hybridized sense and antisense strand, in which the antisense strand and/or the sense strand may include one or more of the modifications described herein. The antisense strand may include modifications at the 3′ end and/or the 5′ end and/or at one or more positions that occur 1-6 (e.g., 1-5, 1-4, 1-3, 1-2) nucleotides from either end of the strand. The sense strand may include modifications at the 3′ end and/or the 5′ end and/or at any one of the intervening positions between the two ends of the strand. The iRNA agent may also include a duplex comprising two hybridized antisense strands. The first and/or the second antisense strand may include one or more of the modifications described herein. Thus, one and/or both antisense strands may include modifications at the 3′ end and/or the 5′ end and/or at one or more positions that occur 1-6 (e.g., 1-5, 1-4, 1-3, 1-2) nucleotides from either end of the strand. Particular configurations are discussed below.

Modifications that can be useful for producing iRNA agents that meet the preferred nuclease resistance criteria delineated above can include one or more of the following chemical and/or stereochemical modifications of the sugar, base, and/or phosphate backbone:
(i) chiral (Sₚ) thioates. Thus, preferred NRMs include nucleotide dimers with an enriched or pure for a particular chiral form of a modified phosphate group containing a heteroatom at the nonbridging position, e.g., Sp or Rp, at the position X, where this is the position normally occupied by the oxygen. The atom at X can also be S, Se, Nr₂, or Br₃. When X is S, enriched or chirally pure Sp linkage is preferred. Enriched means at least 70, 80, 90, 95, or 99% of the preferred form. Such NRMs are discussed in more detail below;
(ii) attachment of one or more cationic groups to the sugar, base, and/or the phosphorus atom of a phosphate or modified phosphate backbone moiety. Thus, preferred NRMs include monomers at the terminal position derivatized at a cationic group. As the 5′ end of an antisense sequence should have a terminal —OH or phosphate group this NRM is preferably not used at the 5′ end of an anti-sense sequence. The group should be attached at a position on the base which minimizes interference with H bond formation and hybridization, e.g., away form the face which interacts with the complementary base on the other strand, e.g, at the 5′ position of a pyrimidine or a 7-position of a purine. These are discussed in more detail below;
(iii) nonphosphate linkages at the termini. Thus, preferred NRMs include Non-phosphate linkages, e.g., a linkage of 4 atoms which confers greater resistance to cleavage than does a phosphate bond. Examples include 3′ CH2—NCH₃—O—CH₂-5′ and 3′ CH₂—NH—(O═)—CH₂-5′;
(iv) 3′-bridging thiophosphates and 5′-bridging thiophosphates. Thus, preferred NRM's can included these structures;
(v) L-RNA, 2′-5′ linkages, inverted linkages, a-nucleosides. Thus, other preferred NRM's include: L nucleosides and dimeric nucleotides derived from L-nucleosides; 2′-5′ phosphate, non-phosphate and modified phosphate linkages (e.g., thiophosphates, phosphoramidates and boronophosphates); dimers having inverted linkages, e.g., 3′-3′ or 5′-5′ linkages; monomers having an alpha linkage at the 1′ site on the sugar, e.g., the structures described herein having an alpha linkage;
(vi) conjugate groups. Thus, preferred NRM's can include, e.g., a targeting moiety or a conjugated ligand described herein conjugated with the monomer, e.g., through the sugar, base, or backbone;
(vi) abasic linkages. Thus, preferred NRM's can include an abasic monomer, e.g., an abasic monomer as described herein (e.g., a nucleobaseless monomer); an aromatic or heterocyclic or polyheterocyclic aromatic monomer as described herein.; and
(vii) 5′-phosphonates and 5′-phosphate prodrugs. Thus, preferred NRM's include monomers, preferably at the terminal position, e.g., the 5′ position, in which one or more atoms of the phosphate group is derivatized with a protecting group, which protecting group or groups, are removed as a result of the action of a component in the subject's body, e.g, a carboxyesterase or an enzyme present in the subject's body. E.g., a phosphate prodrug in which a carboxy esterase cleaves the protected molecule resulting in the production of a thioate anion which attacks a carbon adjacent to the O of a phosphate and resulting in the production of an unprotected phosphate.

One or more different NRM modifications can be introduced into an iRNA agent or into a sequence of an iRNA agent. An NRM modification can be used more than once in a sequence or in an iRNA agent. As some NRMs interfere with hybridization the total number incorporated, should be such that acceptable levels of iRNA agent duplex formation are maintained.

In some embodiments NRM modifications are introduced into the terminal cleavage site or in the cleavage region of a sequence (a sense strand or sequence) which does not target a desired sequence or gene in the subject. This can reduce off-target silencing.

Chiral Sₚ Thioates

A modification can include the alteration, e.g., replacement, of one or both of the non-linking (X and Y) phosphate oxygens and/or of one or more of the linking (W and Z) phosphate oxygens. Formula X below depicts a phosphate moiety linking two sugar/sugar surrogate-base moieties, SB₁ and SB₂.

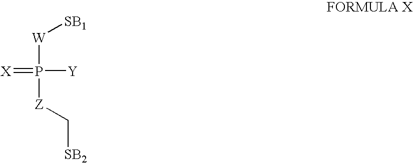

In certain embodiments, one of the non-linking phosphate oxygens in the phosphate backbone moiety (X and Y) can be replaced by any one of the following: S, Se, BR₃ (R is hydrogen, alkyl, aryl, etc.), C (i.e., an alkyl group, an aryl group, etc.), H, NR₂ (R is hydrogen alkyl, aryl, etc.), or OR (R is alkyl or aryl). The phosphorus atom in an unmodified phosphate group is achiral. However, replacement of one of the non-linking oxygens with one of the above atoms or groups of atoms renders the phosphorus atom chiral; in other words a phosphorus atom in a phosphate group modified in this way is a stereogenic center. The stereogenic phosphorus atom can possess either the “R” configuration (herein R_{P}) or the “S” configuration (herein Sₚ). Thus if 60% of a population of stereogenic phosphorus atoms have the Rₚ configuration, then the remaining 40% of the population of stereogenic phosphorus atoms have the Sₚ configuration.

In some embodiments, iRNA agents, having phosphate groups in which a phosphate non-linking oxygen has been replaced by another atom or group of atoms, may contain a population of stereogenic phosphorus atoms in which at least about 50% of these atoms (e.g., at least about 60% of these atoms, at least about 70% of these atoms, at least about 80% of these atoms, at least about 90% of these atoms, at least about 95% of these atoms, at least about 98% of these atoms, at least about 99% of these atoms) have the Sₚ configuration. Alternatively, iRNA agents having phosphate groups in which a phosphate non-linking oxygen has been replaced by another atom or group of atoms may contain a population of stereogenic phosphorus atoms in which at least about 50% of these atoms (e.g., at least about 60% of these atoms, at least about 70% of these atoms, at least about 80% of these atoms, at least about 90% of these atoms, at least about 95% of these atoms, at least about 98% of these atoms, at least about 99% of these atoms) have the Rₚ configuration. In other embodiments, the population of stereogenic phosphorus atoms may have the Sₚ configuration and may be substantially free of stereogenic phosphorus atoms having the Rₚ configuration. In still other embodiments, the population of stereogenic phosphorus atoms may have the Rₚ configuration and may be substantially free of stereogenic phosphorus atoms having the Sₚ configuration. As used herein, the phrase “substantially free of stereogenic phosphorus atoms having the Rₚ configuration” means that moieties containing stereogenic phosphorus atoms having the Rₚ configuration cannot be detected by conventional methods known in the art (chiral HPLC, ¹H NMR analysis using chiral shift reagents, etc.). As used herein, the phrase “substantially free of stereogenic phosphorus atoms having the Sₚ configuration” means that moieties containing stereogenic phosphorus atoms having the Sₚ configuration cannot be detected by conventional methods known in the art (chiral HPLC, ¹H NMR analysis using chiral shift reagents, etc.).

In a preferred embodiment, modified iRNA agents contain a phosphorothioate group, i.e., a phosphate groups in which a phosphate non-linking oxygen has been replaced by a sulfur atom. In an especially preferred embodiment, the population of phosphorothioate stereogenic phosphorus atoms may have the Sₚ configuration and be substantially free of stereogenic phosphorus atoms having the Rₚ configuration.

Phosphorothioates may be incorporated into iRNA agents using dimers e.g., formulas X-1 and X-2. The former can be used to introduce phosphorothioate 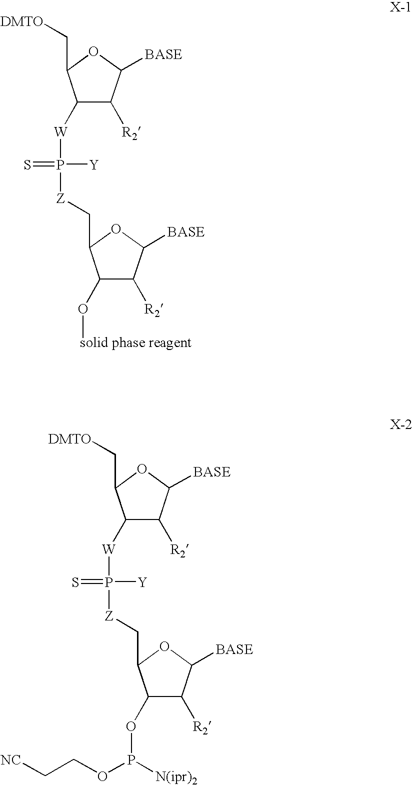
at the 3′ end of a strand, while the latter can be used to introduce this modification at the 5′ end or at a position that occurs e.g., 1, 2, 3, 4, 5, or 6 nucleotides from either end of the strand. In the above formulas, Y can be 2-cyanoethoxy, W and Z can be O, R₂′ can be, e.g., a substituent that can impart the C-3 endo configuration to the sugar (e.g., OH, F, OCH₃), DMT is dimethoxytrityl, and “BASE” can be a natural, unusual, or a universal base.

X-1 and X-2 can be prepared using chiral reagents or directing groups that can result in phosphorothioate-containing dimers having a population of stereogenic phosphorus atoms having essentially only the Rₚ configuration (i.e., being substantially free of the Sₚ configuration) or only the Sₚ configuration (i.e., being substantially free of the Rₚ configuration). Alternatively, dimers can be prepared having a population of stereogenic phosphorus atoms in which about 50% of the atoms have the Rₚ configuration and about 50% of the atoms have the Sₚ configuration. Dimers having stereogenic phosphorus atoms with the Rₚ configuration can be identified and separated from dimers having stereogenic phosphorus atoms with the Sₚ configuration using e.g., enzymatic degradation and/or conventional chromatography techniques.

Cationic Groups

Modifications can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); or NH(CH₂CH₂NH)ₙCH₂CH₂-AMINE (AMINE=NH2; alkylamino, dialkylamino, heterocyclyl, arylamino, diaryl amino, heteroaryl amino, or diheteroaryl amino).

Nonphosphate Linkages

Modifications can also include the incorporation of nonphosphate linkages at the 5′ and/or 3′ end of a strand. Examples of nonphosphate linkages which can replace the phosphate group include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methyl phosphonate and hydroxylamino groups.

### 3′-Bridging Thiophosphates and 5′-Bridging Thiophosphates; Locked-RNA, 2′-5′ Linkages, Inverted Linkages, α-Nucleosides; Conjugate Groups; Abasic Linkages; and 5′-Phosphonates and 5′-Phosphate Prodrugs

Referring to formula X above, modifications can include replacement of one of the bridging or linking phosphate oxygens in the phosphate backbone moiety (W and Z). Unlike the situation where only one of X or Y is altered, the phosphorus center in the phosphorodithioates is achiral which precludes the formation of iRNA agents containing a stereogenic phosphorus atom.

Modifications can also include linking two sugars via a phosphate or modified phosphate group through the 2′ position of a first sugar and the 5′ position of a second sugar. Also contemplated are inverted linkages in which both a first and second sugar are each linked through the respective 3′ positions. Modified RNA's can also include “abasic” sugars, which lack a nucleobase at C-1′. The sugar group can also contain one or more carbons that possess the opposite stereochemical configuration than that of the corresponding carbon in ribose. Thus, a modified iRNA agent can include nucleotides containing e.g., arabinose, as the sugar. In another subset of this modification, the natural, unusual, or universal base may have the α-configuration. Modifications can also include L-RNA.

Modifications can also include 5′-phosphonates, e.g., P(O)(O⁻)₂—X—C^{5′}-sugar (X═CH2, CF2, CHF and 5′-phosphate prodrugs, e.g., P(O)[OCH2CH2SC(O)R]₂CH₂C^{5′}-sugar. In the latter case, the prodrug groups may be decomposed via reaction first with carboxy esterases. The remaining ethyl thiolate group via intramolecular S_{N}2 displacement can depart as episulfide to afford the underivatized phosphate group.

Modification can also include the addition of conjugating groups described elsewhere herein, which are preferably attached to an iRNA agent through any amino group available for conjugation.

Nuclease resistant modifications include some which can be placed only at the terminus and others which can go at any position. Generally the modifications that can inhibit hybridization so it is preferably to use them only in terminal regions, and preferable to not use them at the cleavage site or in the cleavage region of an sequence which targets a subject sequence or gene. The can be used anywhere in a sense sequence, provided that sufficient hybridization between the two sequences of the iRNA agent is maintained. In some embodiments it is desirable to put the NRM at the cleavage site or in the cleavage region of a sequence which does not target a subject sequence or gene, as it can minimize off-target silencing.

In addition, an iRNA agent described herein can have an overhang which does not form a duplex structure with the other sequence of the iRNA agent—it is an overhang, but it does hybridize, either with itself, or with another nucleic acid, other than the other sequence of the iRNA agent.

In most cases, the nuclease-resistance promoting modifications will be distributed differently depending on whether the sequence will target a sequence in the subject (often referred to as an anti-sense sequence) or will not target a sequence in the subject (often referred to as a sense sequence). If a sequence is to target a sequence in the subject, modifications which interfere with or inhibit endonuclease cleavage should not be inserted in the region which is subject to RISC mediated cleavage, e.g., the cleavage site or the cleavage region (As described in Elbashir et al., 2001, Genes and Dev. 15: 188, hereby incorporated by reference). Cleavage of the target occurs about in the middle of a 20 or 21 nt guide RNA, or about 10 or 11 nucleotides upstream of the first nucleotide which is complementary to the guide sequence. As used herein cleavage site refers to the nucleotide on either side of the cleavage site, on the target or on the iRNA agent strand which hybridizes to it. Cleavage region means an nucleotide with 1, 2, or 3 nucleotides of the cleave site, in either direction.)

Such modifications can be introduced into the terminal regions, e.g., at the terminal position or with 2, 3, 4, or 5 positions of the terminus, of a sequence which targets or a sequence which does not target a sequence in the subject.

An iRNA agent can have a first and a second strand chosen from the following:
a first strand which does not target a sequence and which has an NRM modification at or within 1, 2, 3, 4, 5, or 6 positions from the 3′ end;
a first strand which does not target a sequence and which has an NRM modification at or within 1, 2, 3, 4, 5, or 6 positions from the 5′ end;
a first strand which does not target a sequence and which has an NRM modification at or within 1, 2, 3, 4, 5, or 6 positions from the 3′ end and which has a NRM modification at or within 1, 2, 3, 4, 5, or 6 positions from the 5′ end;
a first strand which does not target a sequence and which has an NRM modification at the cleavage site or in the cleavage region;
a first strand which does not target a sequence and which has an NRM modification at the cleavage site or in the cleavage region and one or more of an NRM modification at or within 1, 2, 3, 4, 5, or 6 positions from the 3′ end, a NRM modification at or within 1, 2, 3, 4, 5, or 6 positions from the 5′ end, or NRM modifications at or within 1, 2, 3, 4, 5, or 6 positions from both the 3′ and the 5′ end; and
a second strand which targets a sequence and which has an NRM modification at or within 1, 2, 3, 4, 5, or 6 positions from the 3′ end;
a second strand which targets a sequence and which has an NRM modification at or within 1, 2, 3, 4, 5, or 6 positions from the 5′ end (5′ end NRM modifications are preferentially not at the terminus but rather at a position 1, 2, 3, 4, 5, or 6 away from the 5′ terminus of an antisense strand);
a second strand which targets a sequence and which has an NRM modification at or within 1, 2, 3, 4, 5, or 6 positions from the 3′ end and which has a NRM modification at or within 1, 2, 3, 4, 5, or 6 positions from the 5′ end;
a second strand which targets a sequence and which preferably does not have an NRM modification at the cleavage site or in the cleavage region;
a second strand which targets a sequence and which does not have an NRM modification at the cleavage site or in the cleavage region and one or more of an NRM modification at or within 1, 2, 3, 4, 5, or 6 positions from the 3′ end, a NRM modification at or within 1, 2, 3, 4, 5, or 6 positions from the 5′ end, or NRM modifications at or within 1, 2, 3, 4, 5, or 6 positions from both the 3′ and the 5′ end(5′ end NRM modifications are preferentially not at the terminus but rather at a position 1, 2, 3, 4, 5, or 6 away from the 5′ terminus of an antisense strand).

An iRNA agent can also target two sequences and can have a first and second strand chosen from:
a first strand which targets a sequence and which has an NRM modification at or within 1, 2, 3, 4, 5, or 6 positions from the 3′ end;
a first strand which targets a sequence and which has an NRM modification at or within 1, 2, 3, 4, 5, or 6 positions from the 5′ end (5′ end NRM modifications are preferentially not at the terminus but rather at a position 1, 2, 3, 4, 5, or 6 away from the 5′ terminus of an antisense strand);
a first strand which targets a sequence and which has an NRM modification at or within 1, 2, 3, 4, 5, or 6 positions from the 3′ end and which has a NRM modification at or within 1, 2, 3, 4, 5, or 6 positions from the 5′ end;
a first strand which targets a sequence and which preferably does not have an NRM modification at the cleavage site or in the cleavage region;
a first strand which targets a sequence and which dose not have an NRM modification at the cleavage site or in the cleavage region and one or more of an NRM modification at or within 1, 2, 3, 4, 5, or 6 positions from the 3′ end, a NRM modification at or within 1, 2, 3, 4, 5, or 6 positions from the 5′ end, or NRM modifications at or within 1, 2, 3, 4, 5, or 6 positions from both the 3′ and the 5′ end (5′ end NRM modifications are preferentially not at the terminus but rather at a position 1, 2, 3, 4, 5, or 6 away from the 5′ terminus of an antisense strand) and a second strand which targets a sequence and which has an NRM modification at or within 1, 2, 3, 4, 5, or 6 positions from the 3′ end;
a second strand which targets a sequence and which has an NRM modification at or within 1, 2, 3, 4, 5, or 6 positions from the 5′ end (5′ end NRM modifications are preferentially not at the terminus but rather at a position 1, 2, 3, 4, 5, or 6 away from the 5′ terminus of an antisense strand);
a second strand which targets a sequence and which has an NRM modification at or within 1, 2, 3, 4, 5, or 6 positions from the 3′ end and which has a NRM modification at or within 1, 2, 3, 4, 5, or 6 positions from the 5′ end;
a second strand which targets a sequence and which preferably does not have an NRM modification at the cleavage site or in the cleavage region;
a second strand which targets a sequence and which dose not have an NRM modification at the cleavage site or in the cleavage region and one or more of an NRM modification at or within 1, 2, 3, 4, 5, or 6 positions from the 3′ end, a NRM modification at or within 1, 2, 3, 4, 5, or 6 positions from the 5′ end, or NRM modifications at or within 1, 2, 3, 4, 5, or 6 positions from both the 3′ and the 5′ end (5′ end NRM modifications are preferentially not at the terminus but rather at a position 1, 2, 3, 4, 5, or 6 away from the 5′ terminus of an antisense strand).
   Ribose Mimics

An RNA, e.g., an iRNA agent, can incorporate a ribose mimic. In addition, the invention includes iRNA agents having a ribose mimic and another element described herein. E.g., the invention includes an iRNA agent described herein, e.g., an iRNA agent having a modification on the sense strand to inhibit off-target silencing, an iRNA agent having a non canonical pairing, an iRNA agent having an architecture or structure described herein, an iRNA associated with an amphipathic delivery agent described herein, an iRNA associated with a drug delivery module described herein, an iRNA agent administered as described herein, or an iRNA agent formulated as described herein, which also incorporates a ribose mimic.

Thus, an aspect of the invention features an iRNA agent that includes a secondary hydroxyl group, which can increase efficacy and/or confer nuclease resistance to the agent. Nucleases, e.g., cellular nucleases, can hydrolyze nucleic acid phosphodiester bonds, resulting in partial or complete degradation of the nucleic acid. The secondary hydroxy group confers nuclease resistance to an iRNA agent by rendering the iRNA agent less prone to nuclease degradation relative to an iRNA which lacks the modification. While not wishing to be bound by theory, it is believed that the presence of a secondary hydroxyl group on the iRNA agent can act as a structural mimic of a 3′ ribose hydroxyl group, thereby causing it to be less susceptible to degradation.

The secondary hydroxyl group refers to an “OH” radical that is attached to a carbon atom substituted by two other carbons and a hydrogen. The secondary hydroxyl group that confers nuclease resistance as described above can be part of any acyclic carbon-containing group. The hydroxyl may also be part of any cyclic carbon-containing group, and preferably one or more of the following conditions is met (1) there is no ribose moiety between the hydroxyl group and the terminal phosphate group or (2) the hydroxyl group is not on a sugar moiety which is coupled to a base. The hydroxyl group is located at least two bonds (e.g., at least three bonds away, at least four bonds away, at least five bonds away, at least six bonds away, at least seven bonds away, at least eight bonds away, at least nine bonds away, at least ten bonds away, etc.) from the terminal phosphate group phosphorus of the iRNA agent. In preferred embodiments, there are five intervening bonds between the terminal phosphate group phosphorus and the secondary hydroxyl group.

Preferred iRNA agent delivery modules with five intervening bonds between the terminal phosphate group phosphorus and the secondary hydroxyl group have the following structure (see formula Y below):

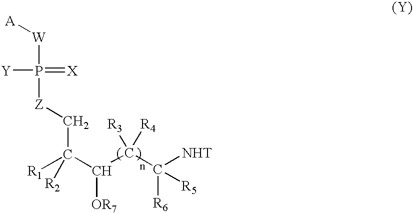

Referring to formula Y, A is an iRNA agent, including any iRNA agent described herein. The iRNA agent may be connected directly or indirectly (e.g., through a spacer or linker) to “W” of the phosphate group. These spacers or linkers can include e.g., —(CH₂)ₙ—, —(CH₂)ₙN—, —(CH₂)ₙO—, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents.

The iRNA agents can have a terminal phosphate group that is unmodified (e.g., W, X, Y, and Z are O) or modified. In a modified phosphate group, W and Z can be independently NH, O, or S; and X and Y can be independently S, Se, BH₃⁻, C₁-C₆ alkyl, C₆-C₁₀ aryl, H, O, O⁻, alkoxy or amino (including alkylamino, arylamino, etc.). Preferably, W, X and Z are O and Y is S.

R₁ and R₃ are each, independently, hydrogen; or C₁-C₁₀₀ alkyl, optionally substituted with hydroxyl, amino, halo, phosphate or sulfate and/or may be optionally inserted with N, O, S, alkenyl or alkynyl.

R₂ is hydrogen; C₁-C₁₀₀ alkyl, optionally substituted with hydroxyl, amino, halo, phosphate or sulfate and/or may be optionally inserted with N, O, S, alkenyl or alkynyl; or, when n is 1, R₂ may be taken together with R₄ or R₆ to form a ring of 5-12 atoms.

R₄ is hydrogen; C₁-C₁₀₀ alkyl, optionally substituted with hydroxyl, amino, halo, phosphate or sulfate and/or may be optionally inserted with N, O, S, alkenyl or alkynyl; or, when n is 1, R₄ may be taken together with R₂ or R₅ to form a ring of 5-12 atoms.

R₅ is hydrogen, C₁-C₁₀₀ alkyl optionally substituted with hydroxyl, amino, halo, phosphate or sulfate and/or may be optionally inserted with N, O, S, alkenyl or alkynyl; or, when n is 1, R₅ may be taken together with R₄ to form a ring of 5-12 atoms.

R₆ is hydrogen, C₁-C₁₀₀ alkyl, optionally substituted with hydroxyl, amino, halo, phosphate or sulfate and/or may be optionally inserted with N, O, S, alkenyl or alkynyl, or, when n is 1, R₆ may be taken together with R₂ to form a ring of 6-10 atoms;
R₇ is hydrogen, C₁-C₁₀₀ alkyl, or C(O)(CH₂)_{q}C(O)NHR₉; T is hydrogen or a functional group; n and q are each independently 1-100; R₈ is C₁-C₁₀ alkyl or C₆-C₁₀ aryl; and R₉ is hydrogen, C1-C10 alkyl, C6-C10 aryl or a solid support agent.

Preferred embodiments may include one of more of the following subsets of iRNA agent delivery modules.

In one subset of RNAi agent delivery modules, A can be connected directly or indirectly through a terminal 3′ or 5′ ribose sugar carbon of the RNA agent.

In another subset of RNAi agent delivery modules, X, W, and Z are O and Y is S.

In still yet another subset of RNAi agent delivery modules, n is 1, and R₂ and R₆ are taken together to form a ring containing six atoms and R₄ and R₅ are taken together to form a ring containing six atoms. Preferably, the ring system is a trans-decalin. For example, the RNAi agent delivery module of this subset can include a compound of Formula (Y-1):

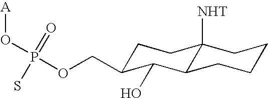

The functional group can be, for example, a targeting group (e.g., a steroid or a carbohydrate), a reporter group (e.g., a fluorophore), or a label (an isotopically labeled moiety). The targeting group can further include protein binding agents, endothelial cell targeting groups (e.g., RGD peptides and mimetics), cancer cell targeting groups (e.g., folate Vitamin B12, Biotin), bone cell targeting groups (e.g., bisphosphonates, polyglutamates, polyaspartates), multivalent mannose (for e.g., macrophage testing), lactose, galactose, N-acetyl-galactosamine, monoclonal antibodies, glycoproteins, lectins, melanotropin, or thyrotropin.

As can be appreciated by the skilled artisan, methods of synthesizing the compounds of the formulae herein will be evident to those of ordinary skill in the art. The synthesized compounds can be separated from a reaction mixture and further purified by a method such as column chromatography, high pressure liquid chromatography, or recrystallization. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Ribose Replacement Monomer Subunits

iRNA agents can be modified in a number of ways which can optimize one or more characteristics of the iRNA agent. An RNA agent, e.g., an iRNA agent can include a ribose replacement monomer subunit (RRMS), such as those described herein In addition, an iRNA agent can have an RRMS and another element described herein. E.g., the invention includes an iRNA agent described herein, e.g., an iRNA agent having a modification on the sense strand to inhibit off-target silencing, an iRNA agent having a non canonical pairing, an iRNA agent having an architecture or structure described herein, an iRNA associated with an amphipathic delivery agent described herein, an iRNA associated with a drug delivery module described herein, an iRNA agent administered as described herein, or an iRNA agent formulated as described herein, which also incorporates a RRMS.

The ribose sugar of one or more ribonucleotide subunits of an iRNA agent can be replaced with another moiety, e.g., a non-carbohydrate (preferably cyclic) carrier. A ribonucleotide subunit in which the ribose sugar of the subunit has been so replaced is referred to herein as an RRMS. A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The carriers further include (i) at least two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” as used herein refers to a constituent ring atom of the cyclic carrier, e.g., a carbon atom or a heteroatom (distinct from an atom which provides a backbone attachment point), that connects a selected moiety. The moiety can be, e.g., a ligand, e.g., a targeting or delivery moiety, or a moiety which alters a physical property, e.g., lipophilicity, of an iRNA agent. Optionally, the selected moiety is connected by an intervening tether to the cyclic carrier. Thus, it will include a functional group, e.g., an amino group, or generally, provide a bond, that is suitable for incorporation or tethering of another chemical entity, e.g., a ligand to the constituent ring.

Incorporation of one or more RRMSs described herein into an RNA agent, e.g., an iRNA agent, particularly when tethered to an appropriate entity, can confer one or more new properties to the RNA agent and/or alter, enhance or modulate one or more existing properties in the RNA molecule. E.g., it can alter one or more of lipophilicity or nuclease resistance. Incorporation of one or more RRMSs described herein into an iRNA agent can, particularly when the RRMS is tethered to an appropriate entity, modulate, e.g., increase, binding affinity of an iRNA agent to a target mRNA, change the geometry of the duplex form of the iRNA agent, alter distribution or target the iRNA agent to a particular part of the body, or modify the interaction with nucleic acid binding proteins (e.g., during RISC formation and strand separation).

Accordingly, in one aspect, the invention features, an iRNA agent preferably comprising a first strand and a second strand, wherein at least one subunit having a formula (R-1) is incorporated into at least one of said strands.

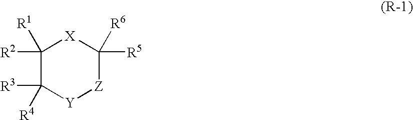

Referring to formula (R-1), X is N(CO)R⁷, NR⁷ or CH₂; Y is NR⁸, O, S, CR⁹R¹⁰, or absent; and Z is CR¹¹R¹² or absent.

Each of R¹, R², R³, R⁴, R⁹, and R¹⁰ is, independently, H, OR^{a}, OR^{b}, (CH₂)ₙOR^{a}, or (CH₂)ₙOR^{b}, provided that at least one of R¹, R², R³, R⁴, R₉, and R₁₀ is OR^{a} or OR^{b} and that at least one of R¹, R², R³, R⁴, R⁹, and R¹⁰ is (CH₂)ₙOR^{a}, or (CH₂)ₙOR^{b} (when the RRMS is terminal, one of R¹, R², R³, R⁴, R⁹, and R¹⁰ will include R^{a} and one will include R^{b}; when the RRMS is internal, two of R¹, R², R³, R⁴, R⁹, and R¹⁰ will each include an R^{b}); further provided that preferably OR^{a} may only be present with (CH₂)ₙOR^{b} and (CH₂)ₙOR^{a} may only be present with OR^{b}.

Each of R⁵, R⁶, R¹¹, and R¹² is, independently, H, C₁-C₆ alkyl optionally substituted with 1-3 R¹³, or C(O)NHR⁷; or R⁵ and R¹¹ together are C₃-C₈ cycloalkyl optionally substituted with R¹⁴.

R⁷ is C₁-C₂₀ alkyl substituted with NR^{c}R^{d}; R⁸ is C₁-C₆ alkyl; R¹³ is hydroxy, C₁-C₄ alkoxy, or halo; and R¹⁴ is NR^{c}R⁷.

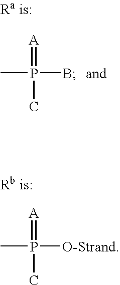

Each of A and C is, independently, O or S.

B is OH, O⁻, or

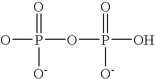

R^{c} is H or C1-C6 alkyl; R^{d} is H or a ligand; and n is 1-4.

In a preferred embodiment the ribose is replaced with a pyrroline scaffold, and X is N(CO)R⁷ or NR⁷, Y is CR⁹R¹⁰, and Z is absent.

In other preferred embodiments the ribose is replaced with a piperidine scaffold, and X is N(CO)R⁷ or NR⁷, Y is CR⁹R¹⁰, and Z is CR¹¹R¹².

In other preferred embodiments the ribose is replaced with a piperazine scaffold, and X is N(CO)R⁷ or NR⁷, Y is NR⁸, and Z is CR¹¹R¹².

In other preferred embodiments the ribose is replaced with a morpholino scaffold, and X is N(CO)R⁷ or NR⁷, Y is O, and Z is CR¹¹R¹².

In other preferred embodiments the ribose is replaced with a decalin scaffold, and X is CH₂; Y is CR⁹R¹⁰; and Z is CR¹¹R¹²; and R⁵ and R¹¹ together are C⁶ cycloalkyl.

In other preferred embodiments the ribose is replaced with a decalin/indane scaffold and, and X is CH₂; Y is CR⁹R¹⁰; and Z is CR¹¹R¹²; and R⁵ and R¹¹ together are C⁵ cycloalkyl.

In other preferred embodiments, the ribose is replaced with a hydroxyproline scaffold.

RRMSs described herein may be incorporated into any double-stranded RNA-like molecule described herein, e.g., an iRNA agent. An iRNA agent may include a duplex comprising a hybridized sense and antisense strand, in which the antisense strand and/or the sense strand may include one or more of the RRMSs described herein. An RRMS can be introduced at one or more points in one or both strands of a double-stranded iRNA agent. An RRMS can be placed at or near (within 1, 2, or 3 positions) of the 3′ or 5′ end of the sense strand or at near (within 2 or 3 positions of) the 3′ end of the antisense strand. In some embodiments it is preferred to not have an RRMS at or near (within 1, 2, or 3 positions of) the 5′ end of the antisense strand. An RRMS can be internal, and will preferably be positioned in regions not critical for antisense binding to the target.

In an embodiment, an iRNA agent may have an RRMS at (or within 1, 2, or 3 positions of) the 3′ end of the antisense strand. In an embodiment, an iRNA agent may have an RRMS at (or within 1, 2, or 3 positions of) the 3′ end of the antisense strand and at (or within 1, 2, or 3 positions of) the 3′ end of the sense strand. In an embodiment, an iRNA agent may have an RRMS at (or within 1, 2, or 3 positions of) the 3′ end of the antisense strand and an RRMS at the 5′ end of the sense strand, in which both ligands are located at the same end of the iRNA agent.

In certain embodiments, two ligands are tethered, preferably, one on each strand and are hydrophobic moieties. While not wishing to be bound by theory, it is believed that pairing of the hydrophobic ligands can stabilize the iRNA agent via intermolecular van der Waals interactions.

In an embodiment, an iRNA agent may have an RRMS at (or within 1, 2, or 3 positions of) the 3′ end of the antisense strand and an RRMS at the 5′ end of the sense strand, in which both RRMSs may share the same ligand (e.g., cholic acid) via connection of their individual tethers to separate positions on the ligand. A ligand shared between two proximal RRMSs is referred to herein as a “hairpin ligand.”

In other embodiments, an iRNA agent may have an RRMS at the 3′ end of the sense strand and an RRMS at an internal position of the sense strand. An iRNA agent may have an RRMS at an internal position of the sense strand; or may have an RRMS at an internal position of the antisense strand; or may have an RRMS at an internal position of the sense strand and an RRMS at an internal position of the antisense strand.

In preferred embodiments the iRNA agent includes a first and second sequence, which are preferably two separate molecules as opposed to two sequences located on the same strand, have sufficient complementarity to each other to hybridize (and thereby form a duplex region), e.g., under physiological conditions, e.g., under physiological conditions but not in contact with a helicase or other unwinding enzyme.

It is preferred that the first and second sequences be chosen such that the ds iRNA agent includes a single strand or unpaired region at one or both ends of the molecule. Thus, a ds iRNA agent contains first and second sequences, preferable paired to contain an overhang, e.g., one or two 5′ or 3′ overhangs but preferably a 3′ overhang of 2-3 nucleotides. Most embodiments will have a 3′ overhang. Preferred sRNA agents will have single-stranded overhangs, preferably 3′ overhangs, of 1 or preferably 2 or 3 nucleotides in length at each end. The overhangs can be the result of one strand being longer than the other, or the result of two strands of the same length being staggered. 5′ ends are preferably phosphorylated.

Tethered Entities

A wide variety of entities can be tethered to an iRNA agent, e.g., to the carrier of an RRMS. Examples are described below in the context of an RRMS but that is only preferred, entities can be coupled at other points to an iRNA agent.

Preferred moieties are ligands, which are coupled, preferably covalently, either directly or indirectly via an intervening tether, to the RRMS carrier. In preferred embodiments, the ligand is attached to the carrier via an intervening tether. As discussed above, the ligand or tethered ligand may be present on the RRMS monomer when the RRMS monomer is incorporated into the growing strand. In some embodiments, the ligand may be incorporated into a “precursor” RRMS after a “precursor” RRMS monomer has been incorporated into the growing strand. For example, an RRMS monomer having, e.g., an amino-terminated tether (i.e., having no associated ligand), e.g., TAP-(CH₂)ₙNH₂ may be incorporated into a growing sense or antisense strand. In a subsequent operation, i.e., after incorporation of the precursor monomer into the strand, a ligand having an electrophilic group, e.g., a pentafluorophenyl ester or aldehyde group, can subsequently be attached to the precursor RRMS by coupling the electrophilic group of the ligand with the terminal nucleophilic group of the precursor RRMS tether.

In preferred embodiments, a ligand alters the distribution, targeting or lifetime of an iRNA agent into which it is incorporated. In preferred embodiments a ligand provides an enhanced affinity for a selected target, e.g, molecule, cell or cell type, compartment, e.g., a cellular or organ compartment, tissue, organ or region of the body, as, e.g., compared to a species absent such a ligand. For example, in a preferred embodiment, a ligand will provide enhanced selectivity to a neural cell, such as in the brain. Preferred ligands will not take part in duplex airing in a duplexed nucleic acid.

Preferred ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a neural cell.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP. In one embodiment, a ligand can facilitate the movement of the iRNA agent across the blood-brain barrier.

Ligands can be proteins, e.g., glycoproteins, or peptides, e.g., molecules having a specific affinity for a co-ligand, or antibodies e.g., an antibody, that binds to a specified cell type such as a neural cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose.

The ligand can be a substance, e.g, a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the iRNA agent into the cell by activating an inflammatory response, for example. Exemplary ligands that would have such an effect include tumor necrosis factor alpha (TNFalpha), interleukin-1 beta, or gamma interferon.

In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-liver target tissue of the body. Preferably, the target tissue is the brain. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the liver and therefore less likely to be cleared from the body.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long (see Table 10, for example).

TABLE 10

Exemplary Cell Permeation Peptides

| | | |
|---|---|---|
| Cell | | |
| Permeation | | |
| Peptide | Amino acid Sequence | Reference |

| | | | |
|---|---|---|---|
| Penetratin | RQIKIWFQNRRMKWKK | (SEQ ID NO:24) | Derossi et al., J. Biol. |
| | | | Chem. 269:10444, |
| | | | 1994 |
| Tat fragment | GRKKRRQRRRPPQC | (SEQ ID NO:25) | Vives et al., J. Biol. |
| (48-60) | | | Chem., 272:16010, |
| | | | 1997 |
| Signal | GALFLGWLGAAGSTMGAWSQPKKKRKV | (SEQ ID NO:26) | Chaloin et al., |
| Sequence- | | | Biochem. Biophys. |
| based peptide | | | Res. Commun., |
| | | | 243:601, 1998 |
| PVEC | LLIILRRRIRKQAHAHSK | (SEQ ID NO:27) | Elmquist et al., Exp. |
| | | | Cell Res., 269:237, |
| | | | 2001 |
| Transportan | GWTLNSAGYLLKINLKALAALAKKIL | (SEQ ID NO:28) | Pooga et al., FASEB |
| | | | J., 12:67, 1998 |
| Amphiphilic | KLALKLALKALKAALKLA | (SEQ ID NO:29) | Oehlke et al., Mol. |
| model peptide | | | Ther., 2:339, 2000 |
| Arg₉ | RRRRRRRRR | (SEQ ID NO:30) | Mitchell et al., J. |
| | | | Pept. Res., 56:318, |
| | | | 2000 |
| Bacterial cell | KFFKFFKFFK | (SEQ ID NO:31) | |
| wall | | | |
| permeating | | | |
| LL-37 | LLGDFFRKSKEKIGKEFKRIVQRIKDFLRN | (SEQ ID NO:32) | |
| | LVPRTES | | |
| Cecropin P1 | SWLSKTAKKLENSAKKRISEGIAIAIQGGPR | (SEQ ID NO:33) | |
| α-defensin | ACYCRIPACIAGERRYGTCIYQGRLWAFCC | (SEQ ID NO:34) | |
| b-defensin | DHYNCVSSGGQCLYSACPIFTKIQGTCYR | (SEQ ID NO:35) | |
| | GKAKCCK | | |
| Bactenecin | RKCRIVVIRVCR | (SEQ ID NO:36) | |
| PR-39 | RRRPRPPYLPRPRPPPFFPPRLPPRIPPGFPP | (SEQ ID NO:37) | |
| | RFPPRFPGKR-NH2 | | |
| Indolicidin | ILPWKWPWWPWRR-NH2 | (SEQ ID NO:38) | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO:39). An RFGF analogue (e.g., amino acid sequence AALLPVLLAAP (SEQ ID NO:40) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO:41) and the Drosophila Antennapedia protein (RQIKIWFQNRRMKWKK (SEQ ID NO:42) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature 354:82-84, 1991). Preferably the peptide or peptidomimetic tethered to an iRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

A “cell permeation peptide” is capable of permeating a cell, e.g., α-mammalian cell, such as a human cell. A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

In one embodiment, a targeting peptide tethered to an RRMS can be an amphipathic α-helical peptide. Exemplary amphipathic α-helical peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins. A number of factors will preferably be considered to maintain the integrity of helix stability. For example, a maximum number of helix stabilization residues will be utilized (e.g., leu, ala, or lys), and a minimum number helix destabilization residues will be utilized (e.g., proline, or cyclic monomeric units. The capping residue will be considered (for example Gly is an exemplary N-capping residue and/or C-terminal amidation can be used to provide an extra H-bond to stabilize the helix. Formation of salt bridges between residues with opposite charges, separated by i±3, or i±4 positions can provide stability. For example, cationic residues such as lysine, arginine, homo-arginine, ornithine or histidine can form salt bridges with the anionic residues glutamate or aspartate.

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides.

Methods for Making iRNA Agents

iRNA agents can include modified or non-naturally occurring bases, e.g., bases described herein. In addition, iRNA agents can have a modified or non-naturally occurring base and another element described herein. E.g., the invention includes an iRNA agent described herein, e.g., an iRNA agent having a modification on the sense strand to inhibit off-target silencing, an iRNA agent having a non canonical pairing, an iRNA agent having an architecture or structure described herein, an iRNA associated with an amphipathic delivery agent described herein, an iRNA associated with a drug delivery module described herein, an iRNA agent administered as described herein, or an iRNA agent formulated as described herein, which also incorporates a modified or non-naturally occurring base.

The synthesis and purification of oligonucleotide peptide conjugates can be performed by established methods. See, for example, Trufert et al., Tetrahedron, 52:3005, 1996; and Manoharan, “Oligonucleotide Conjugates in Antisense Technology,” in Antisense Drug Technology, ed. S. T. Crooke, Marcel Dekker, Inc., 2001.

In one embodiment of the invention, a peptidomimetic can be modified to create a constrained peptide that adopts a distinct and specific preferred conformation, which can increase the potency and selectivity of the peptide. For example, the constrained peptide can be an azapeptide (Gante, Synthesis 405-413, 1989). An azapeptide is synthesized by replacing the α-carbon of an amino acid with a nitrogen atom without changing the structure of the amino acid side chain. For example, the azapeptide can be synthesized by using hydrazine in traditional peptide synthesis coupling methods, such as by reacting hydrazine with a “carbonyl donor,” e.g., phenylchloroformate.

In one embodiment of the invention, a peptide or peptidomimetic (e.g., a peptide or peptidomimetic tethered to an RRMS) can be an N-methyl peptide. N-methyl peptides are composed of N-methyl amino acids, which provide an additional methyl group in the peptide backbone, thereby potentially providing additional means of resistance to proteolytic cleavage. N-methyl peptides can by synthesized by methods known in the art (see, for example, Lindgren et al., Trends Pharmacol. Sci. 21:99, 2000; Cell Penetrating Peptides: Processes and Applications, Langel, ed., CRC Press, Boca Raton, Fla., 2002; Fische et al., Bioconjugate. Chem. 12: 825, 2001; Wander et al., J. Am. Chem. Soc., 124:13382, 2002). For example, an Ant or Tat peptide can be an N-methyl peptide.

In one embodiment of the invention, a peptide or peptidomimetic (e.g., a peptide or peptidomimetic tethered to an RRMS) can be a β-peptide. β-peptides form stable secondary structures such as helices, pleated sheets, turns and hairpins in solutions. Their cyclic derivatives can fold into nanotubes in the solid state. β-peptides are resistant to degradation by proteolytic enzymes. β-peptides can be synthesized by methods known in the art. For example, an Ant or Tat peptide can be a β-peptide.

In one embodiment of the invention, a peptide or peptidomimetic (e.g., a peptide or peptidomimetic tethered to an RRMS) can be a oligocarbamate. Oligocarbamate peptides are internalized into a cell by a transport pathway facilitated by carbamate transporters. For example, an Ant or Tat peptide can be an oligocarbamate.

In one embodiment of the invention, a peptide or peptidomimetic (e.g., a peptide or peptidomimetic tethered to an RRMS) can be an oligourea conjugate (or an oligothiourea conjugate), in which the amide bond of a peptidomimetic is replaced with a urea moiety. Replacement of the amide bond provides increased resistance to degradation by proteolytic enzymes, e.g., proteolytic enzymes in the gastrointestinal tract. In one embodiment, an oligourea conjugate is tethered to an iRNA agent for use in oral delivery. The backbone in each repeating unit of an oligourea peptidomimetic can be extended by one carbon atom in comparison with the natural amino acid. The single carbon atom extension can increase peptide stability and lipophilicity, for example. An oligourea peptide can therefore be advantageous when an iRNA agent is directed for passage through a bacterial cell wall, or when an iRNA agent must traverse the blood-brain barrier, such as for the treatment of a neurological disorder. In one embodiment, a hydrogen bonding unit is conjugated to the oligourea peptide, such as to create an increased affinity with a receptor. For example, an Ant or Tat peptide can be an oligourea conjugate (or an oligothiourea conjugate).

The dsRNA peptide conjugates of the invention can be affiliated with, e.g., tethered to, RRMSs occurring at various positions on an iRNA agent. For example, a peptide can be terminally conjugated, on either the sense or the antisense strand, or a peptide can be bisconjugated (one peptide tethered to each end, one conjugated to the sense strand, and one conjugated to the antisense strand). In another option, the peptide can be internally conjugated, such as in the loop of a short hairpin iRNA agent. In yet another option, the peptide can be affiliated with a complex, such as a peptide-carrier complex.

A peptide-carrier complex consists of at least a carrier molecule, which can encapsulate one or more iRNA agents (such as for delivery to a biological system and/or a cell), and a peptide moiety tethered to the outside of the carrier molecule, such as for targeting the carrier complex to a particular tissue or cell type. A carrier complex can carry additional targeting molecules on the exterior of the complex, or fusogenic agents to aid in cell delivery. The one or more iRNA agents encapsulated within the carrier can be conjugated to lipophilic molecules, which can aid in the delivery of the agents to the interior of the carrier.

A carrier molecule or structure can be, for example, a micelle, a liposome (e.g., a cationic liposome), a nanoparticle, a microsphere, or a biodegradable polymer. A peptide moiety can be tethered to the carrier molecule by a variety of linkages, such as a disulfide linkage, an acid labile linkage, a peptide-based linkage, an oxyamino linkage or a hydrazine linkage. For example, a peptide-based linkage can be a GFLG peptide. Certain linkages will have particular advantages, and the advantages (or disadvantages) can be considered depending on the tissue target or intended use. For example, peptide based linkages are stable in the blood stream but are susceptible to enzymatic cleavage in the lysosomes.

Definitions

The term “halo” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive)carbon atoms in it. The term “haloalkyl” refers to an alkyl in which one or more hydrogen atoms are replaced by halo, and includes alkyl moieties in which all hydrogens have been replaced by halo (e.g., perfluoroalkyl). Alkyl and haloalkyl groups may be optionally inserted with O, N, or S. The terms “aralkyl” refers to an alkyl moiety in which an alkyl hydrogen atom is replaced by an aryl group. Aralkyl includes groups in which more than one hydrogen atom has been replaced by an aryl group. Examples of “aralkyl” include benzyl, 9-fluorenyl, benzhydryl, and trityl groups.

The term “alkenyl” refers to a straight or branched hydrocarbon chain containing 2-8 carbon atoms and characterized in having one or more double bonds. Examples of a typical alkenyl include, but not limited to, allyl, propenyl, 2-butenyl, 3-hexenyl and 3-octenyl groups. The term “alkynyl” refers to a straight or branched hydrocarbon chain containing 2-8 carbon atoms and characterized in having one or more triple bonds. Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The term “alkoxy” refers to an —O-alkyl radical. The term “aminoalkyl” refers to an alkyl substituted with an amino. The term “mercapto” refers to an —SH radical. The term “thioalkoxy” refers to an —S-alkyl radical.

The term “alkylene” refers to a divalent alkyl (i.e., —R—), e.g., —CH₂—, —CH₂CH₂—, and —CH₂CH₂CH₂—. The term “alkylenedioxo” refers to a divalent species of the structure —O—R—O—, in which R represents an alkylene.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons, wherein any ring atom capable of substitution can be substituted by a substituent. The cycloalkyl groups herein described may also contain fused rings. Fused rings are rings that share a common carbon-carbon bond. Examples of cycloalkyl moieties include, but are not limited to, cyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution can be substituted by a substituent. The heterocyclyl groups herein described may also contain fused rings. Fused rings are rings that share a common carbon-carbon bond. Examples of heterocyclyl include, but are not limited to tetrahydrofuranyl, tetrahydropyranyl, piperidinyl, morpholino, pyrrolinyl and pyrrolidinyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution can be substituted by a substituent.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino(alkyl, aryl, aralkyl), S(O)nalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide(mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The terms “adeninyl, cytosinyl, guaninyl, thyminyl, and uracilyl” and the like refer to radicals of adenine, cytosine, guanine, thyrnine, and uracil.

As used herein, an “unusual” nucleobase can include any one of the following:

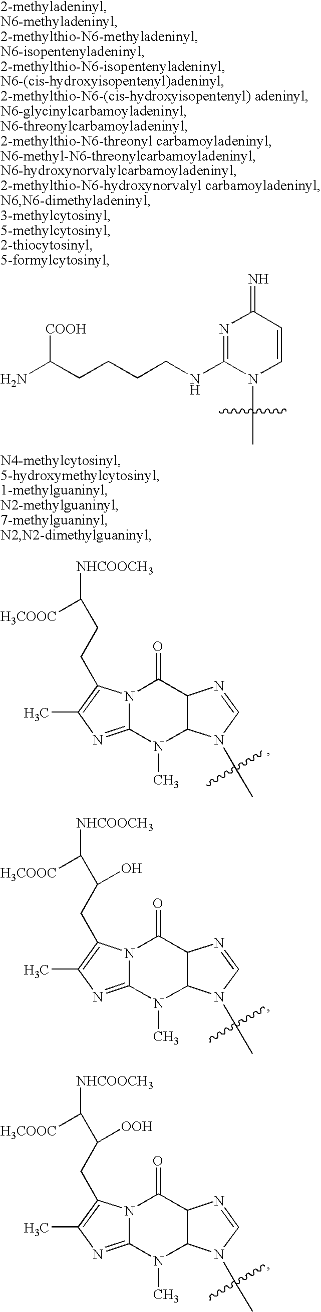

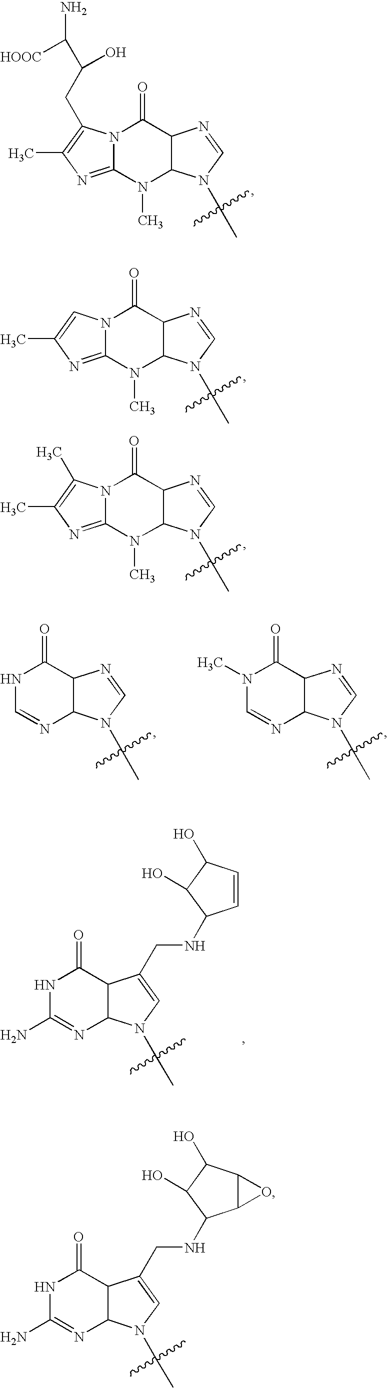

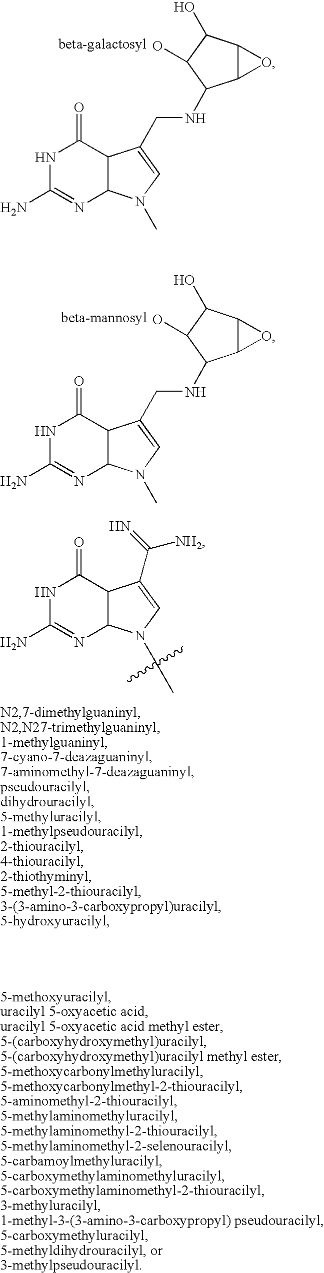

Other than Canonical Watson-Crick Duplex Structures

An RNA, e.g., an iRNA agent can include monomers that can form other than a canonical Watson-Crick pairing with another monomer, e.g., a monomer on another strand. The use of “other than canonical Watson-Crick pairing” between monomers of a duplex can be used to control, often to promote, melting of all or part of a duplex. The iRNA agent can include a monomer at a selected or constrained position that results in a first level of stability in the iRNA agent duplex (e.g., between the two separate molecules of a double stranded iRNA agent) and a second level of stability in a duplex between a sequence of an iRNA agent and another sequence molecule, e.g., a target or off-target sequence in a subject. In some cases the second duplex has a relatively greater level of stability, e.g., in a duplex between an anti-sense sequence of an iRNA agent and a target mRNA. In this case one or more of the monomers, the position of the monomers in the iRNA agent, and the target sequence (sometimes referred to herein as the selection or constraint parameters), are selected such that the iRNA agent duplex has a comparatively lower free energy of association (which while not wishing to be bound by mechanism or theory, is believed to contribute to efficacy by promoting disassociation of the duplex iRNA agent in the context of the RISC) while the duplex formed between an antisense targeting sequence and its target sequence, has a relatively higher free energy of association (which while not wishing to be bound by mechanism or theory, is believed to contribute to efficacy by promoting association of the antisense sequence and the target RNA).

In other cases the second duplex has a relatively lower level of stability, e.g., in a duplex between a sense sequence of an iRNA agent and an off-target mRNA. In this case one or more of the monomers, the position of the monomers in the iRNA agent, and an off-target sequence, are selected such that the iRNA agent duplex is has a comparatively higher free energy of association while the duplex formed between a sense targeting sequence and its off-target sequence, has a relatively lower free energy of association (which while not wishing to be bound by mechanism or theory, is believed to reduce the level of off-target silencing by promoting disassociation of the duplex formed by the sense strand and the off-target sequence).

Thus, inherent in the structure of the iRNA agent is the property of having a first stability for the intra-iRNA agent duplex and a second stability for a duplex formed between a sequence from the iRNA agent and another RNA, e.g., a target mRNA. As discussed above, this can be accomplished by judicious selection of one or more of the monomers at a selected or constrained position, the selection of the position in the duplex to place the selected or constrained position, and selection of the sequence of a target sequence (e.g., the particular region of a target gene which is to be targeted). The iRNA agent sequences which satisfy these requirements are sometimes referred to herein as constrained sequences. Exercise of the constraint or selection parameters can be, e.g., by inspection or by computer assisted methods. Exercise of the parameters can result in selection of a target sequence and of particular monomers to give a desired result in terms of the stability, or relative stability, of a duplex.

Thus, in another aspect, the invention features an iRNA agent which includes: a first sequence which targets a first target region and a second sequence which targets a second target region. The first and second sequences have sufficient complementarity to each other to hybridize, e.g., under physiological conditions, e.g., under physiological conditions but not in contact with a helicase or other unwinding enzyme. In a duplex region of the iRNA agent, at a selected or constrained position, the first target region has a first monomer, and the second target region has a second monomer. The first and second monomers occupy complementary or corresponding positions. One, and preferably both monomers are selected such that the stability of the pairing of the monomers contribute to a duplex between the first and second sequence will differ form the stability of the pairing between the first or second sequence with a target sequence.

Usually, the monomers will be selected (selection of the target sequence may be required as well) such that they form a pairing in the iRNA agent duplex which has a lower free energy of dissociation, and a lower Tm, than will be possessed by the paring of the monomer with its complementary monomer in a duplex between the iRNA agent sequence and a target RNA duplex.

The constraint placed upon the monomers can be applied at a selected site or at more than one selected site. By way of example, the constraint can be applied at more than 1, but less than 3, 4, 5, 6, or 7 sites in an iRNA agent duplex.

A constrained or selected site can be present at a number of positions in the iRNA agent duplex. E.g., a constrained or selected site can be present within 3, 4, 5, or 6 positions from either end, 3′ or 5′ of a duplexed sequence. A constrained or selected site can be present in the middle of the duplex region, e.g., it can be more than 3, 4, 5, or 6, positions from the end of a duplexed region.

In some embodiment the duplex region of the iRNA agent will have mismatches, in addition to the selected or constrained site or sites. Preferably it will have no more than 1, 2, 3, 4, or 5 bases, which do not form canonical Watson-Crick pairs or which do not hybridize. Overhangs are discussed in detail elsewhere herein but are preferably about 2 nucleotides in length. The overhangs can be complementary to the gene sequences being targeted or can be other sequence. TT is a preferred overhang sequence. The first and second iRNA agent sequences can also be joined, e.g., by additional bases to form a hairpin, or by other non-base linkers.

The monomers can be selected such that: first and second monomers are naturally occurring ribonucleotides, or modified ribonucleotides having naturally occurring bases, and when occupying complemetary sites either do not pair and have no substantial level of H-bonding, or form a non canonical Watson-Crick pairing and form a non-canonical pattern of H bonding, which usually have a lower free energy of dissociation than seen in a canonical Watson-Crick pairing, or otherwise pair to give a free energy of association which is less than that of a preselected value or is less, e.g., than that of a canonical pairing. When one (or both) of the iRNA agent sequences duplexes with a target, the first (or second) monomer forms a canonical Watson-Crick pairing with the base in the complemetary position on the target, or forms a non-canonical Watson-Crick pairing having a higher free energy of dissociation and a higher Tm than seen in the pairing in the iRNA agent. The classical Watson-Crick parings are as follows: A-T, G-C, and A-U. Non-canonical Watson-Crick pairings are known in the art and can include, U-U, G-G, G-Aₜᵣₐₙₛ, G-A_{cis}, and GU.

The monomer in one or both of the sequences is selected such that, it does not pair, or forms a pair with its corresponding monomer in the other sequence which minimizes stability (e.g., the H bonding formed between the monomer at the selected site in the one sequence and its monomer at the corresponding site in the other sequence are less stable than the H bonds formed by the monomer one (or both) of the sequences with the respective target sequence. The monomer of one or both strands is also chosen to promote stability in one or both of the duplexes made by a strand and its target sequence. E.g., one or more of the monomers and the target sequences are selected such that at the selected or constrained position, there is are no H bonds formed, or a non canonical pairing is formed in the iRNA agent duplex, or they otherwise pair to give a free energy of association which is less than that of a preselected value or is less, e.g., than that of a canonical pairing, but when one (or both) sequences form a duplex with the respective target, the pairing at the selected or constrained site is a canonical Watson-Crick paring.

The inclusion of such a monomer will have one or more of the following effects: it will destabilize the iRNA agent duplex, it will destabilize interactions between the sense sequence and unintended target sequences, sometimes referred to as off-target sequences, and duplex interactions between the a sequence and the intended target will not be destabilized.

A non-naturally occurring or modified monomer or monomers can be chosen such that when a non-naturally occurring or modified monomer occupies a position at the selected or constrained position in an iRNA agent they exhibit a first free energy of dissociation and when one (or both) of them pairs with a naturally occurring monomer, the pair exhibits a second free energy of dissociation, which is usually higher than that of the pairing of the first and second monomers. E.g., when the first and second monomers occupy complementary positions they either do not pair and have no substantial level of H-bonding, or form a weaker bond than one of them would form with a naturally occurring monomer, and reduce the stability of that duplex, but when the duplex dissociates at least one of the strands will form a duplex with a target in which the selected monomer will promote stability, e.g., the monomer will form a more stable pair with a naturally occurring monomer in the target sequence than the pairing it formed in the iRNA agent.

An example of such a pairing is 2-amino A and either of a 2-thio pyrimidine analog of U or T.

When placed in complementary positions of the iRNA agent these monomers will pair very poorly and will minimize stability. However, a duplex is formed between 2 amino A and the U of a naturally occurring target, or a duplex is between 2-thio U and the A of a naturally occurring target or 2-thio T and the A of a naturally occurring target will have a relatively higher free energy of dissociation and be more stable.

The term “other than canonical Watson-Crick pairing” as used herein, refers to a pairing between a first monomer in a first sequence and a second monomer at the corresponding position in a second sequence of a duplex in which one or more of the following is true: (1) there is essentially no pairing between the two, e.g., there is no significant level of H bonding between the monomers or binding between the monomers does not contribute in any significant way to the stability of the duplex; (2) the monomers are a non-canonical paring of monomers having a naturally occurring bases, i.e., they are other than A-T, A-U, or G-C, and they form monomer-monomer H bonds, although generally the H bonding pattern formed is less strong than the bonds formed by a canonical pairing; or (3) at least one of the monomers includes a non-naturally occurring bases and the H bonds formed between the monomers is, preferably formed is less strong than the bonds formed by a canonical pairing, namely one or more of A-T, A-U, G-C.

The term “off-target” as used herein, refers to as a sequence other than the sequence to be silenced. Universal Bases: “wild-cards”; shape-based complementarity

Bi-stranded, multisite replication of a base pair between difluorotoluene and adenine: confirmation by ‘inverse’ sequencing. Liu, D.; Moran, S.; Kool, E. T. Chem. Biol., 1997, 4, 919-926)

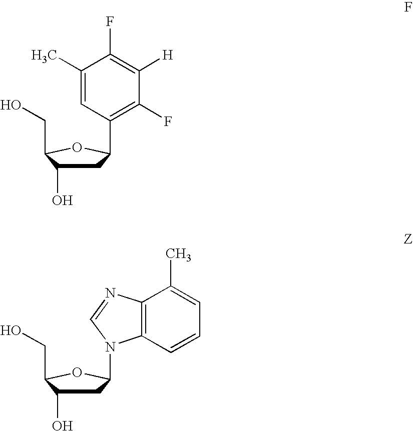

(Importance of terminal base pair hydrogen-bonding in 3′-end proofreading by the Klenow fragment of DNA polymerase I. Morales, J. C.; Kool, E. T. Biochemistry, 2000, 39, 2626-2632)

(Selective and stable DNA base pairing without hydrogen bonds. Matray, T, J.; Kool, E. T. J. Am. Chem. Soc., 1998, 120, 6191-6192)

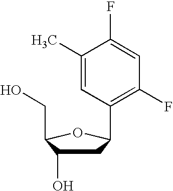

(Difluorotoluene, a nonpolar isostere for thymine, codes specifically and efficiently for adenine in DNA replication. Moran, S. Ren, R. X.-F.; Rumney IV, S.; Kool, E. T. J. Am. Chem. Soc., 1997, 119, 2056-2057)

(Structure and base pairing properties of a replicable nonpolar isostere for deoxyadenosine. Guckian, K. M.; Morales, J. C.; Kool, E. T. J. Org. Chem., 1998, 63, 9652-9656)

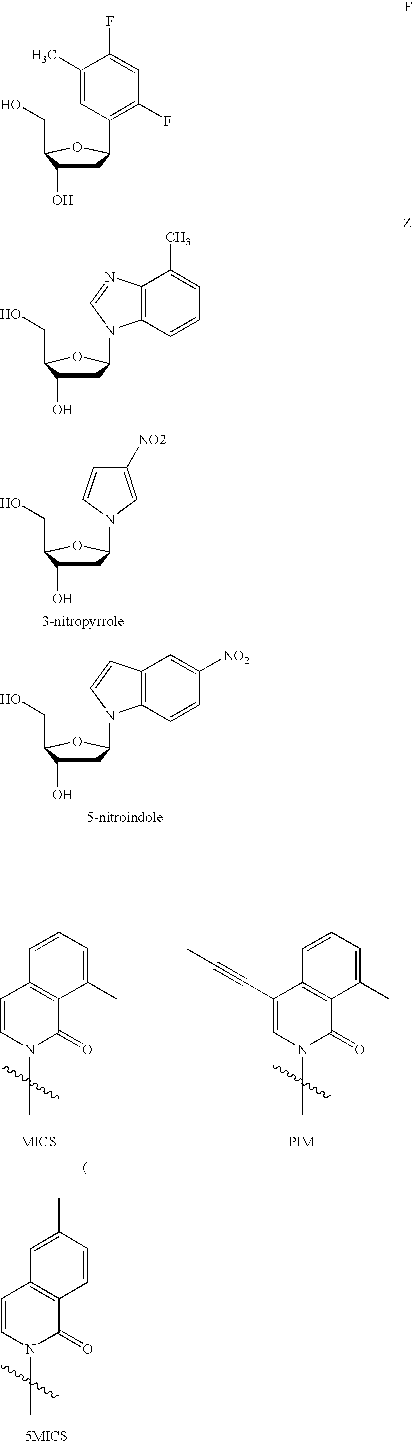

(Universal bases for hybridization, replication and chain termination. Berger, M.; Wu. Y.; Ogawa, A. K.; McMinn, D. L.; Schultz, P.G.; Romesberg, F. E. Nucleic Acids Res., 2000, 28, 2911-2914) 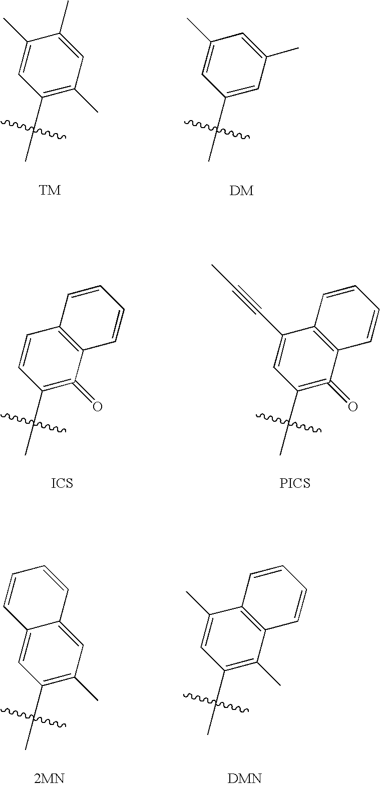 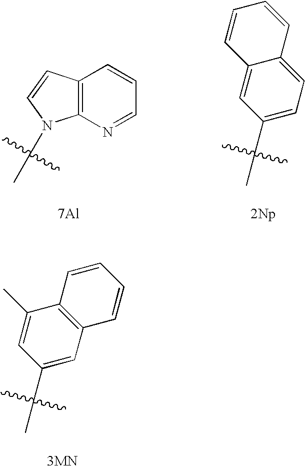
(1. Efforts toward the expansion of the genetic alphabet: Information storage and replication with unnatural hydrophobic base pairs. Ogawa, A. K.; Wu, Y.; McMinn, D. L.; Liu, J.; Schultz, P. G.; Romesberg, F. E. J. Am. Chem. Soc., 2000, 122, 3274-3287. 2. Rational design of an unnatural base pair with increased kinetic selectivity. Ogawa, A. K.; Wu. Y.; Berger, M.; Schultz, P. G.; Romesberg, F. E. J. Am. Chem. Soc., 2000, 122, 8803-8804)

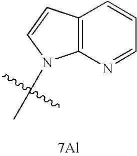

(Efforts toward expansion of the genetic alphabet: replication of DNA with three base pairs. Tae, E. L.; Wu, Y.; Xia, G.; Schultz, P. G.; Romesberg, F. E. J. Am. Chem. Soc., 2001, 123, 7439-7440)

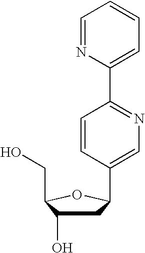

(1. Efforts toward expansion of the genetic alphabet: Optimization of interbase hydrophobic interactions. Wu, Y.; Ogawa, A. K.; Berger, M.; McMinn, D. L.; Schultz, P. G.; Romesberg, F. E. J. Am. Chem. Soc., 2000, 122, 7621-7632. 2. Efforts toward expansion of genetic alphabet: DNA polymerase recognition of a highly stable, self-pairing hydrophobic base. McMinn, D. L.; Ogawa. A. K.; Wu, Y.; Liu, J.; Schultz, P. G.; Romesberg, F. E. J. Am. Chem. Soc., 1999, 121, 11585-11586)

(A stable DNA duplex containing a non-hydrogen-bonding and non-shape complementary base couple: Interstrand stacking as the stability determining factor. Brotschi, C.; Haberli, A.; Leumann, C, J. Angew. Chem. Int. Ed., 2001, 40, 3012-3014)

(2,2′-Bipyridine Ligandoside: A novel building block for modifying DNA with intra-duplex metal complexes. Weizman, H.; Tor, Y. J. Am. Chem. Soc., 2001, 123, 3375-3376)

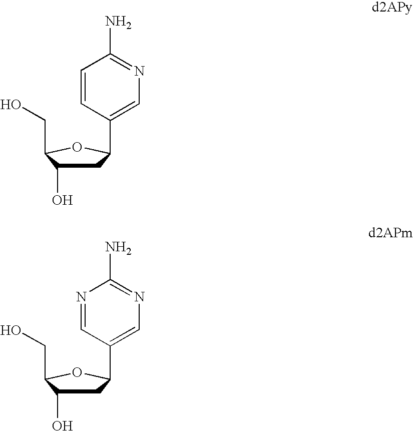

(Minor groove hydration is critical to the stability of DNA duplexes. Lan, T.; McLaughlin, L. W. J. Am. Chem. Soc., 2000, 122, 6512-13)

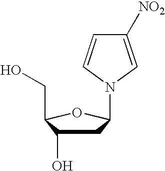

(Effect of the Universal base 3-nitropyrrole on the selectivity of neighboring natural bases. Oliver, J. S.; Parker, K. A.; Suggs, J. W. Organic Lett., 2001, 3, 1977-1980. 2. Effect of the 1-(2′-deoxy-β-D-ribofuranosyl)-3-nitropyrrol residue on the stability of DNA duplexes and triplexes. Amosova, O.; George J.; Fresco, J. R. Nucleic Acids Res., 1997, 25, 1930-1934. 3. Synthesis, structure and deoxyribonucleic acid sequencing with a universal nucleosides: 1-(2′-deoxy-β-D-ribofuranosyl)-3-nitropyrrole. Bergstrom, D. E.; Zhang, P.; Toma, P. H.; Andrews, P. C.; Nichols, R. J. Am. Chem. Soc., 1995, 117, 1201-1209)

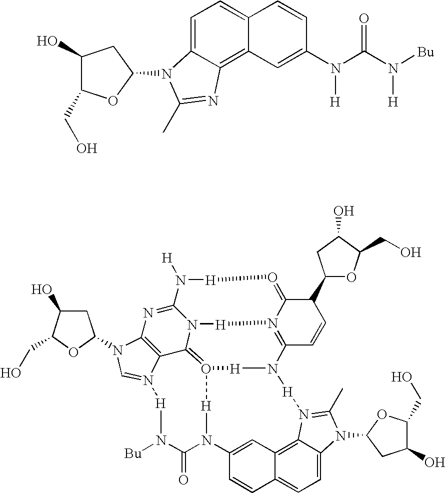

(Model studies directed toward a general triplex DNA recognition scheme: a novel DNA base that binds a CG base-pair in an organic solvent. Zimmerman, S. C.; Schmitt, P. J. Am. Chem. Soc., 1995, 117, 10769-10770)

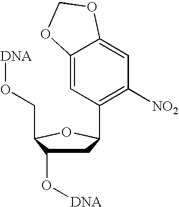

(A universal, photocleavable DNA base: nitropiperonyl 2′-deoxyriboside. J. Org. Chem., 2001, 66, 2067-2071)

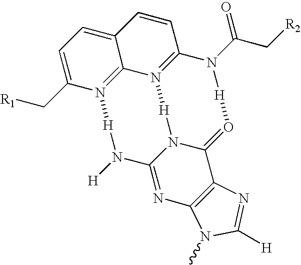

(Recognition of a single guanine bulge by 2-acylamino-1,8-naphthyridine. Nakatani, K.; Sando, S.; Saito, I. J. Am. Chem. Soc., 2000, 122, 2172-2177. b. Specific binding of 2-amino-1,8-naphthyridine into single guanine bulge as evidenced by photooxidation of GC doublet, Nakatani, K.; Sando, S.; Yoshida, K.; Saito, I. Bioorg. Med. Chem. Lett., 2001, 11, 335-337)

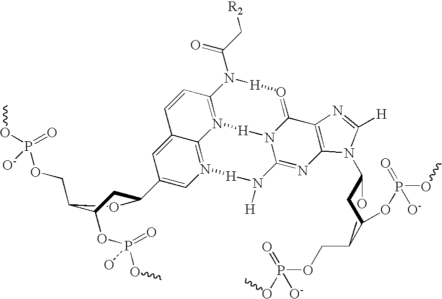

Other universal bases can have the following formulas: 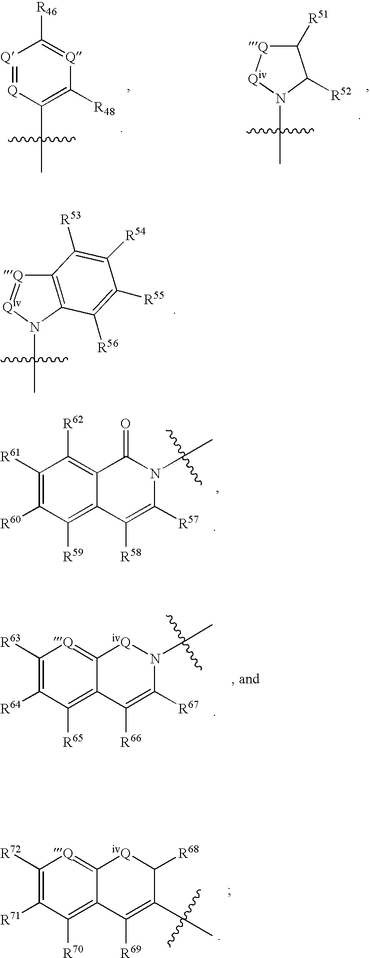
wherein:
Q is N or CR⁴⁴;
Q′ is N or CR⁴⁵;
Q″ is N or CR⁴⁷;
Q′″ is N or CR⁴⁹;
Q^{iv} is N or CR⁵⁰;
R⁴⁴ is hydrogen, halo, hydroxy, nitro, protected hydroxy, NH₂, NHR^{b}, or NR^{b}R^{c}, C₁-C₆ alkyl, C₆-C₁₀ aryl, C₆-C₁₀ heteroaryl, C₃-C₈ heterocyclyl, or when taken together with R⁴⁵ forms —OCH₂O—;
R⁴⁵ is hydrogen, halo, hydroxy, nitro, protected hydroxy, NH₂, NHR^{b}, or NR^{b}R^{c}, C₁-C₆ alkyl, C₆-C₁₀ aryl, C₆-C₁₀ heteroaryl, C₃-C₈ heterocyclyl, or when taken together with R⁴⁴ or R⁴⁶ forms —OCH₂O—;
R⁴⁶ is hydrogen, halo, hydroxy, nitro, protected hydroxy, NH₂, NHR^{b}, or NR^{b}R^{c}, C₁-C₆ alkyl, C₆-C₁₀ aryl, C₆-C₁₀ heteroaryl, C₃-C₈ heterocyclyl, or when taken together with R⁴⁵ or R⁴⁷ forms —OCH₂O—;
R⁴⁷ is hydrogen, halo, hydroxy, nitro, protected hydroxy, NH₂, NHR^{b}, or NR^{b}R^{c}, C₁-C₆ alkyl, C₆-C₁₀ aryl, C₆-C₁₀ heteroaryl, C₃-C₈ heterocyclyl, or when taken together with R⁴⁶ or R⁴⁸ forms —OCH₂O—;
R⁴⁸ is hydrogen, halo, hydroxy, nitro, protected hydroxy, NH₂, NHR^{b}, or NR^{b}R^{c}, C₁-C₆ alkyl, C₆-C₁₀ aryl, C₆-C₁₀ heteroaryl, C₃-C₈ heterocyclyl, or when taken together with R⁴⁷ forms —OCH₂O—;
R⁴⁹R⁵⁰, R⁵¹, R⁵², R⁵³, R⁵⁴, R⁵⁷,R⁵⁸, R⁵⁹, R⁶⁰, R⁶¹, R⁶²,R⁶³, R⁶⁴, R⁶⁵, R⁶⁶, R⁶⁷, R⁶⁸,R⁶⁹ , R⁷⁰, R⁷¹, and R⁷² are each independently selected from hydrogen, halo, hydroxy, nitro, protected hydroxy, NH₂, NHR^{b}, or NR^{b}R^{c}, C₁-C₆ alkyl, C₂-C₆ alkynyl, C₆-C₁₀ aryl, C₆-C₁₀ heteroaryl, C₃-C₈ heterocyclyl, NC(O)R¹⁷, or NC(O)R^{o};
R⁵⁵ is hydrogen, halo, hydroxy, nitro, protected hydroxy, NH₂, NHR^{b}, or NR^{b}R^{c}, C₁-C₆ alkyl, C₂-C₆ alkynyl, C₆-C₁₀ aryl, C₆-C₁₀ heteroaryl, C₃-C₈ heterocyclyl, NC(O)R¹⁷, or NC(O)R^{o}, or when taken together with R⁵⁶ forms a fused aromatic ring which may be optionally substituted;
R⁵⁶ is hydrogen, halo, hydroxy, nitro, protected hydroxy, NH₂, NHR^{b}, or NR^{b}R^{c}, C₁-C₆ alkyl, C₂-C₆ alkynyl, C₆-C₁₀ aryl, C₆-C₁₀ heteroaryl, C₃-C₈ heterocyclyl, NC(O)R¹⁷, or NC(O)R^{o}, or when taken together with R⁵⁵ forms a fused aromatic ring which may be optionally substituted;
R¹⁷ is halo, NH₂, NHR^{b}, or NR^{b}R^{c};
R^{b} is C₁-C₆ alkyl or a nitrogen protecting group;
R^{c} is C₁-C₆ alkyl; and

R^{o} is alkyl optionally substituted with halo, hydroxy, nitro, protected hydroxy, NH₂, NHR^{b}, or NR^{b}R^{c}, C₁-C₆ alkyl, C₂-C₆ alkynyl, C₆-C₁₀ aryl, C₆-C₁₀ heteroaryl, C₃-C₈ heterocyclyl, NC(O)R¹⁷, or NC(O)R^{o}.

Examples of universal bases include:

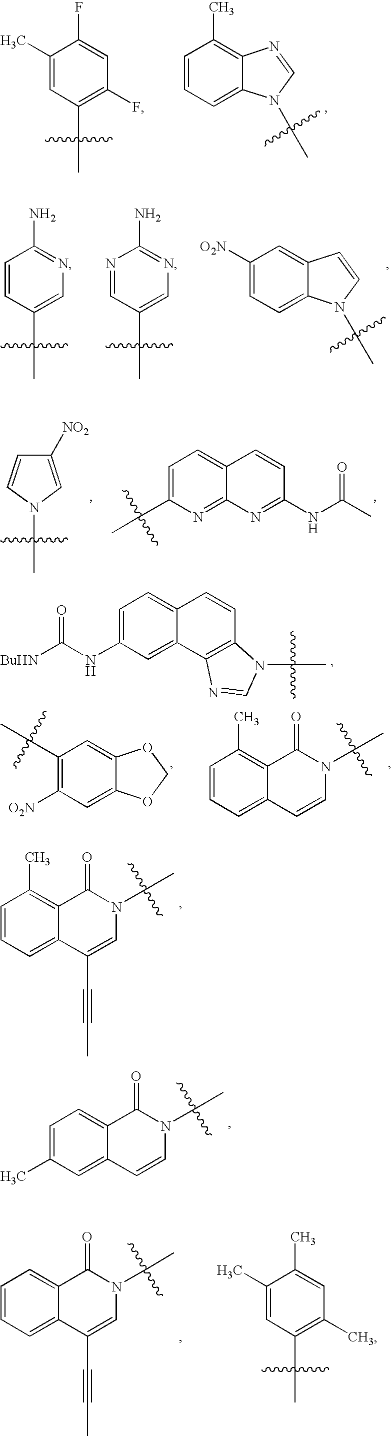

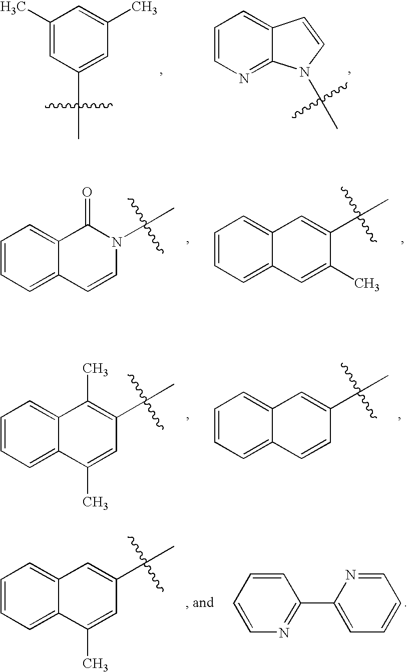

Asymmetrical Modifications

An RNA, e.g., an iRNA agent, can have an asymmetrical modification and another element described herein. E.g., the invention includes an iRNA agent described herein, e.g., an iRNA agent having a modification on the sense strand to inhibit off-target silencing, an iRNA agent having a non canonical pairing, an iRNA agent having an architecture or structure described herein, an iRNA associated with an amphipathic delivery agent described herein, an iRNA associated with a drug delivery module described herein, an iRNA agent administered as described herein, or an iRNA agent formulated as described herein, which also incorporates an asymmetrical modification.

An asymmetrically modified iRNA agent is one in which a strand has a modification which is not present on the other strand. An asymmetrical modification is a modification found on one strand but not on the other strand. Any modification, e.g., any modification described herein, can be present as an asymmetrical modification. An asymmetrical modification can confer any of the desired properties associated with a modification, e.g., those properties discussed herein. E.g., an asymmetrical modification can: confer resistance to degradation, an alteration in half life; target the iRNA agent to a particular target, e.g., to a particular tissue; modulate, e.g., increase or decrease, the affinity of a strand for its complement or target sequence; or hinder or promote modification of a terminal moiety, e.g., modification by a kinase or other enzymes involved in the RISC mechanism pathway. The designation of a modification as having one property does not mean that it has no other property, e.g., a modification referred to as one which promotes stabilization might also enhance targeting.

While not wishing to be bound by theory or any particular mechanistic model, it is believed that asymmetrical modification allows an iRNA agent to be optimized in view of the different or “asymmetrical” functions of the sense and antisense strands. For example, both strands can be modified to increase nuclease resistance, however, since some changes can inhibit RISC activity, these changes can be chosen for the sense stand. In addition, since some modifications, e.g., targeting moieties, can add large bulky groups that, e.g., can interfere with the cleavage activity of the RISC complex, such modifications are preferably placed on the sense strand. Thus, targeting moieties, especially bulky ones (e.g. cholesterol), are preferentially added to the sense strand. In one embodiment, an asymmetrical modification in which a phosphate of the backbone is substituted with S, e.g., a phosphorothioate modification, is present in the antisense strand, and a 2′ modification, e.g., 2′ OMe is present in the sense strand. A targeting moiety can be present at either (or both) the 5′ or 3′ end of the sense strand of the iRNA agent. In a preferred example, a P of the backbone is replaced with S in the antisense strand, 2′OMe is present in the sense strand, and a targeting moiety is added to either the 5′ or 3′ end of the sense strand of the iRNA agent.

In a preferred embodiment an asymmetrically modified iRNA agent has a modification on the sense strand which modification is not found on the antisense strand and the antisense strand has a modification which is not found on the sense strand.

Each strand can include one or more asymmetrical modifications. By way of example: one strand can include a first asymmetrical modification which confers a first property on the iRNA agent and the other strand can have a second asymmetrical modification which confers a second property on the iRNA. E.g., one strand, e.g., the sense strand can have a modification which targets the iRNA agent to a tissue, and the other strand, e.g., the antisense strand, has a modification which promotes hybridization with the target gene sequence.

In some embodiments both strands can be modified to optimize the same property, e.g., to increase resistance to nucleolytic degradation, but different modifications are chosen for the sense and the antisense strands, e.g., because the modifications affect other properties as well. E.g., since some changes can affect RISC activity these modifications are chosen for the sense strand.

In one embodiment, one strand has an asymmetrical 2′ modification, e.g., a 2′ OMe modification, and the other strand has an asymmetrical modification of the phosphate backbone, e.g., a phosphorothioate modification. So, in one embodiment the antisense strand has an asymmetrical 2′ OMe modification and the sense strand has an asymmetrical phosphorothioate modification (or vice versa). In a particularly preferred embodiment, the RNAi agent will have asymmetrical 2′-O alkyl, preferably, 2′-OMe modifications on the sense strand and asymmetrical backbone P modification, preferably a phosphorothioate modification in the antisense strand. There can be one or multiple 2′-OMe modifications, e.g., at least 2, 3, 4, 5, or 6, of the subunits of the sense strand can be so modified. There can be one or multiple phosphorothioate modifications, e.g., at least 2, 3, 4, 5, or 6, of the subunits of the antisense strand can be so modified. It is preferable to have an iRNA agent wherein there are multiple 2′-OMe modifications on the sense strand and multiple phophorothioate modifications on the antisense strand. All of the subunits on one or both strands can be so modified. A particularly preferred embodiment of multiple asymmetric modifications on both strands has a duplex region about 20-21, and preferably 19, subunits in length and one or two 3′ overhangs of about 2 subunits in length.

Asymmetrical modifications are useful for promoting resistance to degradation by nucleases, e.g., endonucleases. iRNA agents can include one or more asymmetrical modifications which promote resistance to degradation. In preferred embodiments the modification on the antisense strand is one which will not interfere with silencing of the target, e.g., one which will not interfere with cleavage of the target. Most if not all sites on a strand are vulnerable, to some degree, to degradation by endonucleases. One can determine sites which are relatively vulnerable and insert asymmetrical modifications which inhibit degradation. It is often desirable to provide asymmetrical modification of a UA site in an iRNA agent, and in some cases it is desirable to provide the UA sequence on both strands with asymmetrical modification. Examples of modifications which inhibit endonucleolytic degradation can be found herein. Particularly favored modifications include: 2′ modification, e.g., provision of a 2′ OMe moiety on the U, especially on a sense strand; modification of the backbone, e.g., with the replacement of an O with an S, in the phosphate backbone, e.g., the provision of a phosphorothioate modification, on the U or the A or both, especially on an antisense strand; replacement of the U with a C5 amino linker; replacement of the A with a G (sequence changes are preferred to be located on the sense strand and not the antisense strand); and modification of the at the 2′, 6′, 7′, or 8′ position. Preferred embodiments are those in which one or more of these modifications are present on the sense but not the antisense strand, or embodiments where the antisense strand has fewer of such modifications.

Asymmetrical modification can be used to inhibit degradation by exonucleases. Asymmetrical modifications can include those in which only one strand is modified as well as those in which both are modified. In preferred embodiments the modification on the antisense strand is one which will not interfere with silencing of the target, e.g., one which will not interfere with cleavage of the target. Some embodiments will have an asymmetrical modification on the sense strand, e.g., in a 3′ overhang, e.g., at the 3′ terminus, and on the antisense strand, e.g., in a 3′ overhang, e.g., at the 3′ terminus. If the modifications introduce moieties of different size it is preferable that the larger be on the sense strand. If the modifications introduce moieties of different charge it is preferable that the one with greater charge be on the sense strand.

Examples of modifications which inhibit exonucleolytic degradation can be found herein. Particularly favored modifications include: 2′ modification, e.g., provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); modification of the backbone, e.g., with the replacement of a P with an S, e.g., the provision of a phosphorothioate modification, or the use of a methylated P in a 3′ overhang, e.g., at the 3′ terminus; combination of a 2′ modification, e.g., provision of a 2′ 0 Me moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrolidine in a 3′ overhang, e.g., at the 3′ terminus; modification with naproxene, ibuprofen, or other moieties which inhibit degradation at the 3′ terminus. Preferred embodiments are those in which one or more of these modifications are present on the sense but not the antisense strand, or embodiments where the antisense strand has fewer of such modifications.

Modifications, e.g., those described herein, which affect targeting can be provided as asymmetrical modifications. Targeting modifications which can inhibit silencing, e.g., by inhibiting cleavage of a target, can be provided as asymmetrical modifications of the sense strand. A biodistribution altering moiety, e.g., cholesterol, can be provided in one or more, e.g., two, asymmetrical modifications of the sense strand. Targeting modifications which introduce moieties having a relatively large molecular weight, e.g., a molecular weight of more than 400, 500, or 1000 daltons, or which introduce a charged moiety (e.g., having more than one positive charge or one negative charge) can be placed on the sense strand.

Modifications, e.g., those described herein, which modulate, e.g., increase or decrease, the affinity of a strand for its compliment or target, can be provided as asymmetrical modifications. These include: 5 methyl U; 5 methyl C; pseudouridine, Locked nucleic acids include: 2 thio U and 2-amino-A. In some embodiments one or more of these is provided on the antisense strand.

iRNA agents have a defined structure, with a sense strand and an antisense strand, and in many cases short single strand overhangs, e.g., of 2 or 3 nucleotides are present at one or both 3′ ends. Asymmetrical modification can be used to optimize the activity of such a structure, e.g., by being placed selectively within the iRNA. E.g., the end region of the iRNA agent defined by the 5′ end of the sense strand and the 3′ end of the antisense strand is important for function. This region can include the terminal 2, 3, or 4 paired nucleotides and any 3′ overhang. In preferred embodiments asymmetrical modifications which result in one or more of the following are used: modifications of the 5′ end of the sense strand which inhibit kinase activation of the sense strand, including, e.g., attachments of conjugates which target the molecule or the use modifications which protect against 5′ exonucleolytic degradation; or modifications of either strand, but preferably the sense strand, which enhance binding between the sense and antisense strand and thereby promote a “tight” structure at this end of the molecule.

The end region of the iRNA agent defined by the 3′ end of the sense strand and the 5′ end of the antisense strand is also important for function. This region can include the terminal 2, 3, or 4 paired nucleotides and any 3′ overhang. Preferred embodiments include asymmetrical modifications of either strand, but preferably the sense strand, which decrease binding between the sense and antisense strand and thereby promote an “open” structure at this end of the molecule. Such modifications include placing conjugates which target the molecule or modifications which promote nuclease resistance on the sense strand in this region. Modification of the antisense strand which inhibit kinase activation are avoided in preferred embodiments.

Exemplary modifications for asymmetrical placement in the sense strand include the following:
(a) backbone modifications, e.g., modification of a backbone P, including replacement of P with S, or P substituted with alkyl or allyl, e.g., Me, and dithioates (S—P═S); these modifications can be used to promote nuclease resistance;
(b) 2′-O alkyl, e.g., 2′-OMe, 3′-O alkyl, e.g., 3′-OMe (at terminal and/or internal positions); these modifications can be used to promote nuclease resistance or to enhance binding of the sense to the antisense strand, the 3′ modifications can be used at the 5′ end of the sense strand to avoid sense strand activation by RISC;
(c) 2′-5′ linkages (with 2′-H, 2′-OH and 2′-OMe and with P═O or P═S) these modifications can be used to promote nuclease resistance or to inhibit binding of the sense to the antisense strand, or can be used at the 5′ end of the sense strand to avoid sense strand activation by RISC;
(d) L sugars (e.g., L ribose, L-arabinose with 2′-H, 2′-OH and 2′-OMe); these modifications can be used to promote nuclease resistance or to inhibit binding of the sense to the antisense strand, or can be used at the 5′ end of the sense strand to avoid sense strand activation by RISC;
(e) modified sugars (e.g., locked nucleic acids (LNA's), hexose nucleic acids (HNA's) and cyclohexene nucleic acids (CeNA's)); these modifications can be used to promote nuclease resistance or to inhibit binding of the sense to the antisense strand, or can be used at the 5′ end of the sense strand to avoid sense strand activation by RISC;
(f) nucleobase modifications (e.g., C-5 modified pyrimidines, N-2 modified purines, N-7 modified purines, N-6 modified purines), these modifications can be used to promote nuclease resistance or to enhance binding of the sense to the antisense strand;
(g) cationic groups and Zwitterionic groups (preferably at a terminus), these modifications can be used to promote nuclease resistance;
(h) conjugate groups (preferably at terminal positions), e.g., naproxen, biotin, cholesterol, ibuprofen, folic acid, peptides, and carbohydrates; these modifications can be used to promote nuclease resistance or to target the molecule, or can be used at the 5′ end of the sense strand to avoid sense strand activation by RISC.

Exemplary modifications for asymmetrical placement in the antisense strand include the following:
(a) backbone modifications, e.g., modification of a backbone P, including replacement of P with S, or P substituted with alkyl or allyl, e.g., Me, and dithioates (S—P═S);
(b) 2′-O alkyl, e.g., 2′-OMe, (at terminal positions);
(c) 2′-5′ linkages (with 2′-H, 2′-OH and 2′-OMe) e.g., terminal at the 3′ end); e.g., with P═O or P═S preferably at the 3′-end, these modifications are preferably excluded from the 5′ end region as they may interfere with RISC enzyme activity such as kinase activity;
(d) L sugars (e.g, L ribose, L-arabinose with 2′-H, 2′-OH and 2′-OMe); e.g., terminal at the 3′ end; e.g., with P═O or P═S preferably at the 3′-end, these modifications are preferably excluded from the 5′ end region as they may interfere with kinase activity;
(e) modified sugars (e.g., LNA's, HNA's and CeNA's); these modifications are preferably excluded from the 5′ end region as they may contribute to unwanted enhancements of paring between the sense and antisense strands, it is often preferred to have a “loose” structure in the 5′ region, additionally, they may interfere with kinase activity;
(f) nucleobase modifications (e.g., C-5 modified pyrimidines, N-2 modified purines, N-7 modified purines, N-6 modified purines);
(g) cationic groups and Zwitterionic groups (preferably at a terminus);
cationic groups and Zwitterionic groups at 2′-position of sugar; 3′-position of the sugar; as nucleobase modifications (e.g., C-5 modified pyrimidines, N-2 modified purines, N-7 modified purines, N-6 modified purines);
conjugate groups (preferably at terminal positions), e.g., naproxen, biotin, cholesterol, ibuprofen, folic acid, peptides, and carbohydrates, but bulky groups or generally groups which inhibit RISC activity should are less preferred.

The 5′-OH of the antisense strand should be kept free to promote activity. In some preferred embodiments modifications that promote nuclease resistance should be included at the 3′ end, particularly in the 3′ overhang.

In another aspect, the invention features a method of optimizing, e.g., stabilizing, an iRNA agent. The method includes selecting a sequence having activity, introducing one or more asymmetric modifications into the sequence, wherein the introduction of the asymmetric modification optimizes a property of the iRNA agent but does not result in a decrease in activity.

The decrease in activity can be less than a preselected level of decrease. In preferred embodiments decrease in activity means a decrease of less than 5, 10, 20, 40, or 50% activity, as compared with an otherwise similar iRNA lacking the introduced modification. Activity can, e.g., be measured in vivo, or in vitro, with a result in either being sufficient to demonstrate the required maintenance of activity.

The optimized property can be any property described herein and in particular the properties discussed in the section on asymmetrical modifications provided herein. The modification can be any asymmetrical modification, e.g., an asymmetric modification described in the section on asymmetrical modifications described herein. Particularly preferred asymmetric modifications are 2′-O alkyl modifications, e.g., 2′-OMe modifications, particularly in the sense sequence, and modifications of a backbone O, particularly phosphorothioate modifications, in the antisense sequence.

In a preferred embodiment, a sense sequence is selected and provided with an asymmetrical modification, while in other embodiments an antisense sequence is selected and provided with an asymmetrical modification. In some embodiments both sense and antisense sequences are selected and each provided with one or more asymmetrical modifications.

Multiple asymmetric modifications can be introduced into either or both of the sense and antisense sequence. A sequence can have at least 2, 4, 6, 8, or more modifications and all or substantially all of the monomers of a sequence can be modified.

Differential Modification of Terminal Duplex Stability

In one aspect, the invention features an iRNA agent which can have differential modification of terminal duplex stability (DMTDS).

In addition, the invention includes iRNA agents having DMTDS and another element described herein. E.g., the invention includes an iRNA agent described herein, e.g., an iRNA agent having a modification on the sense strand to inhibit off-target silencing, an iRNA agent having a non canonical pairing, an iRNA agent having an architecture or structure described herein, an iRNA associated with an amphipathic delivery agent described herein, an iRNA associated with a drug delivery module described herein, an iRNA agent administered as described herein, or an iRNA agent formulated as described herein, which also incorporates DMTDS.

iRNA agents can be optimized by increasing the propensity of the duplex to disassociate or melt (decreasing the free energy of duplex association), in the region of the 5′ end of the antisense strand duplex. This can be accomplished, e.g., by the inclusion of subunits, which increase the propensity of the duplex to disassociate or melt in the region of the 5′ end of the antisense strand. This can also be accomplished by the attachment of a ligand that increases the propensity of the duplex to disassociate of melt in the region of the 5′ end. While not wishing to be bound by theory, the effect may be due to promoting the effect of an enzyme such as a helicase, for example, promoting the effect of the enzyme in the proximity of the 5′ end of the antisense strand.

The inventors have also discovered that iRNA agents can be optimized by decreasing the propensity of the duplex to disassociate or melt (increasing the free energy of duplex association), in the region of the 3′ end of the antisense strand duplex. This can be accomplished, e.g., by the inclusion of subunits which decrease the propensity of the duplex to disassociate or melt in the region of the 3′ end of the antisense strand. It can also be accomplished by the attachment of ligand that decreases the propensity of the duplex to disassociate or melt in the region of the 5′ end.

Modifications which increase the tendency of the 5′ end of the duplex to dissociate can be used alone or in combination with other modifications described herein, e.g., with modifications which decrease the tendency of the 3′ end of the duplex to dissociate. Likewise, modifications which decrease the tendency of the 3′ end of the duplex to dissociate can be used alone or in combination with other modifications described herein, e.g., with modifications which increase the tendency of the 5′ end of the duplex to dissociate.

Decreasing the Stability of the AS 5′ End of the Duplex

Subunit pairs can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation:

| | | |
|---|---|---|
| A:U | is preferred over | G:C; |
| G:U | is preferred over | G:C; |
| I:C | is preferred over | G:C (I = inosine); |

mismatches, e.g., non-canonical or other than canonical pairings (as described elsewhere herein) are preferred over canonical (A:T, A:U, G:C) pairings;
pairings which include a universal base are preferred over canonical pairings.

A typical ds iRNA agent can be diagrammed as follows:

| | | | | | | | | | | | |
|---|---|---|---|---|---|---|---|---|---|---|---|
| S | | 5′ | | | R₁ | N₁ | N₂ | | N₃ | N₄ | N₅ |
| [N] | | N₋₅ | N₋₄ | N₋₃ | N₋₂ | N₋₁ | R₂ | 3′ | | | |
| AS | 3′ | | | | R₃ | N₁ | N₂ | | N₃ | N₄ | N₅ |
| [N] | | N₋₅ | N₋₄ | N₋₃ | N₋₂ | N₋₁ | R₄ | 5′ | | | |
| S:AS | | | | P₁ | | P₂ | | | P₃ | | P₄ |
| P₅ | | | [N] | | P₋₅ | P₋₄ | P₋₃ | | P₋₂ | P₋₁ | |
| 5′ | | | | | | | | | | | |

S indicates the sense strand; AS indicates antisense strand; R₁ indicates an optional (and nonpreferred) 5′ sense strand overhang; R₂ indicates an optional (though preferred) 3, sense overhang; R₃ indicates an optional (though preferred) 3′ antisense sense overhang; R₄ indicates an optional (and nonpreferred) 5′ antisense overhang; N indicates subunits; [N] indicates that additional subunit pairs may be present; and Pₓ, indicates a paring of sense Nₓ and antisense Nₓ. Overhangs are not shown in the P diagram. In some embodiments a 3′ AS overhang corresponds to region Z, the duplex region corresponds to region X, and the 3′ S strand overhang corresponds to region Y, as described elsewhere herein. (The diagram is not meant to imply maximum or minimum lengths, on which guidance is provided elsewhere herein.)

It is preferred that pairings which decrease the propensity to form a duplex are used at 1 or more of the positions in the duplex at the 5′ end of the AS strand. The terminal pair (the most 5′ pair in terms of the AS strand) is designated as P₁, and the subsequent pairing positions (going in the 3′ direction in terms of the AS strand) in the duplex are designated, P₋₂, P₋₃, P₋₄, P₋₅, and so on. The preferred region in which to modify or modulate duplex formation is at P₋₅ through P₋₁, more preferably P₋₄ through P₋₁, more preferably P₋₃ through P₋₁. Modification at P₋₁, is particularly preferred, alone or with modification(s) other position(s), e.g., any of the positions just identified. It is preferred that at least 1, and more preferably 2, 3, 4, or 5 of the pairs of one of the recited regions be chosen independently from the group of:
A:U
G:U
I:C
mismatched pairs, e.g., non-canonical or other than canonical pairings or pairings which include a universal base.

In preferred embodiments the change in subunit needed to achieve a pairing which promotes dissociation will be made in the sense strand, though in some embodiments the change will be made in the antisense strand.

In a preferred embodiment the at least 2, or 3, of the pairs in P₋₁, through P₋₄, are pairs which promote dissociation.

In a preferred embodiment the at least 2, or 3, of the pairs in P₋₁, through P₋₄, are A:U.

In a preferred embodiment the at least 2, or 3, of the pairs in P₋₁, through P₋₄, are G:U.

In a preferred embodiment the at least 2, or 3, of the pairs in P₋₁, through P₋₄, are I:C.

In a preferred embodiment the at least 2, or 3, of the pairs in P₋₁, through P₋₄, are mismatched pairs, e.g., non-canonical or other than canonical pairings pairings.

In a preferred embodiment the at least 2, or 3, of the pairs in P₋₁, through P₋₄, are pairings which include a universal base.

Increasing the Stability of the AS 3′ End of the Duplex

Subunit pairs can be ranked on the basis of their propensity to promote stability and inhibit dissociation or melting (e.g., on the free energy of association or dissociation of a particular pairing, the simplest approach is to examine the pairs on an individual pair basis, though next neighbor or similar analysis can also be used). In terms of promoting duplex stability:

| | | |
|---|---|---|
| G:C | is preferred over | A:U |

Watson-Crick matches (A:T, A:U, G:C) are preferred over non-canonical or other than canonical pairings
analogs that increase stability are preferred over Watson-Crick matches (A:T, A:U, G:C)
2-amino-A:U is preferred over A:U 2-thio U or 5 Me-thio-U:A are preferred over U:A
G-clamp (an analog of C having 4 hydrogen bonds):G is preferred over C:G
guanadinium-G-clamp:G is preferred over C:G
pseudo uridine:A is preferred over U:A
sugar modifications, e.g., 2′ modifications, e.g., 2° F., ENA, or LNA, which enhance binding are preferred over non-modified moieties and can be present on one or both strands to enhance stability of the duplex. It is preferred that pairings which increase the propensity to form a duplex are used at 1 or more of the positions in the duplex at the 3′ end of the AS strand. The terminal pair (the most 3′ pair in terms of the AS strand) is designated as P₁, and the subsequent pairing positions (going in the 5′ direction in terms of the AS strand) in the duplex are designated, P₂, P₃, P₄, P₅, and so on. The preferred region in which to modify to modulate duplex formation is at P₅ through P₁, more preferably P₄ through P₁, more preferably P₃ through P₁. Modification at P₁, is particularly preferred, alone or with modification(s) at other position(s), e.g., any of the positions just identified. It is preferred that at least 1, and more preferably 2, 3, 4, or 5 of the pairs of the recited regions be chosen independently from the group of:
G:C
a pair having an analog that increases stability over Watson-Crick matches (A:T, A:U, G:C)
2-amino-A:U 2-thio U or 5 Me-thio-U:A
G-clamp (an analog of C having 4 hydrogen bonds):G
guanadinium-G-clamp:G
pseudo uridine:A
a pair in which one or both subunits has a sugar modification, e.g., a 2′ modification, e.g., 2° F., ENA, or LNA, which enhance binding.

In a preferred embodiment the at least 2, or 3, of the pairs in P₋, through P₋, are pairs which promote duplex stability.

In a preferred embodiment the at least 2, or 3, of the pairs in P₁, through P₄, are G:C.

In a preferred embodiment the at least 2, or 3, of the pairs in P₁, through P₄, are a pair having an analog that increases stability over Watson-Crick matches.

In a preferred embodiment the at least 2, or 3, of the pairs in P₁, through P₄, are 2-amino-A:U.

In a preferred embodiment the at least 2, or 3, of the pairs in P₁, through P₄, are 2-thio U or 5 Me-thio-U:A.

In a preferred embodiment the at least 2, or 3, of the pairs in P₁, through P₄, are G-clamp:G.

In a preferred embodiment the at least 2, or 3, of the pairs in P₁, through P₄, are guanidinium-G-clamp:G.

In a preferred embodiment the at least 2, or 3, of the pairs in P₁, through P₄, are pseudo uridine:A.

In a preferred embodiment the at least 2, or 3, of the pairs in P₁, through P₄, are a pair in which one or both subunits has a sugar modification, e.g., a 2′ modification, e.g., 2° F., ENA, or LNA, which enhances binding.

G-clamps and guanidinium G-clamps are discussed in the following references: Holmes and Gait, “The Synthesis of 2′-O-Methyl G-Clamp Containing Oligonucleotides and Their Inhibition of the HIV-1 Tat-TAR Interaction,” Nucleosides, Nucleotides & Nucleic Acids, 22:1259-1262, 2003; Holmes et al., “Steric inhibition of human immunodeficiency virus type-1 Tat-dependent trans-activation in vitro and in cells by oligonucleotides containing 2′-O-methyl G-clamp ribonucleoside analogues,” Nucleic Acids Research, 31:2759-2768, 2003; Wilds, et al., “Structural basis for recognition of guanosine by a synthetic tricyclic cytosine analogue: Guanidinium G-clamp,” Helvetica Chimica Acta, 86:966-978, 2003; Rajeev, et al., “High-Affinity Peptide Nucleic Acid Oligomers Containing Tricyclic Cytosine Analogues,” Organic Letters, 4:4395-4398, 2002; Ausin, et al., “Synthesis of Amino- and Guanidino-G-Clamp PNA Monomers,” Organic Letters, 4:4073-4075, 2002; Maier et al., “Nuclease resistance of oligonucleotides containing the tricyclic cytosine analogues phenoxazine and 9-(2-aminoethoxy)-phenoxazine (“G-clamp”) and origins of their nuclease resistance properties,” Biochemistry, 41:1323-7, 2002; Flanagan, et al., “A cytosine analog that confers enhanced potency to antisense oligonucleotides,” Proceedings Of The National Academy Of Sciences Of The United States Of America, 96:3513-8, 1999.

Simultaneously Decreasing the Stability of the AS 5′End of the Duplex and Increasing the Stability of the AS 3′ End of the Duplex

As is discussed above, an iRNA agent can be modified to both decrease the stability of the AS 5′ end of the duplex and increase the stability of the AS 3′ end of the duplex. This can be effected by combining one or more of the stability decreasing modifications in the AS 5′ end of the duplex with one or more of the stability increasing modifications in the AS 3′ end of the duplex. Accordingly a preferred embodiment includes modification in P₋₅ through P₋₁, more preferably P₋₄ through P₋₁ and more preferably P₋₃ through P₋₁. Modification at P₋₁, is particularly preferred, alone or with other position, e.g., the positions just identified. It is preferred that at least 1, and more preferably 2, 3, 4, or 5 of the pairs of one of the recited regions of the AS 5′ end of the duplex region be chosen independently from the group of:
A:U
G:U
I:C
mismatched pairs, e.g., non-canonical or other than canonical pairings which include a universal base; and
a modification in P₅ through P₁, more preferably P₄ through P₁ and more preferably P₃ through P₁. Modification at P₁, is particularly preferred, alone or with other position, e.g., the positions just identified. It is preferred that at least 1, and more preferably 2, 3, 4, or 5 of the pairs of one of the recited regions of the AS 3′ end of the duplex region be chosen independently from the group of:
G:C
a pair having an analog that increases stability over Watson-Crick matches (A:T, A:U, G:C)
2-amino-A:U 2-thio U or 5 Me-thio-U:A
G-clamp (an analog of C having 4 hydrogen bonds):G
guanadinium-G-clamp:G
pseudo uridine:A
a pair in which one or both subunits has a sugar modification, e.g., a 2′ modification, e.g., 2′F, ENA, or LNA, which enhance binding.

The invention also includes methods of selecting and making iRNA agents having DMTDS. E.g., when screening a target sequence for candidate sequences for use as iRNA agents one can select sequences having a DMTDS property described herein or one which can be modified, preferably with as few changes as possible, especially to the
AS strand, to provide a desired level of DMTDS.

The invention also includes, providing a candidate iRNA agent sequence, and modifying at least one P in P₋₅ through P₋₁ and/or at least one P in P₅ through P₁ to provide a DMTDS iRNA agent.

DMTDS iRNA agents can be used in any method described herein, e.g., to silence a target RNA, in any formulation described herein, and generally in and/or with the methods and compositions described elsewhere herein. DMTDS iRNA agents can incorporate other modifications described herein, e.g., the attachment of targeting agents or the inclusion of modifications which enhance stability, e.g., the inclusion of nuclease resistant monomers or the inclusion of single strand overhangs (e.g., 3′ AS overhangs and/or 3′ S strand overhangs) which self associate to form intrastrand duplex structure.

Preferably these iRNA agents will have an architecture described herein.

### Other Embodiments

An RNA, e.g., an iRNA agent, can be produced in a cell in vivo, e.g., from exogenous DNA templates that are delivered into the cell. For example, the DNA templates can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. The DNA templates, for example, can include two transcription units, one that produces a transcript that includes the top strand of an iRNA agent and one that produces a transcript that includes the bottom strand of an iRNA agent. When the templates are transcribed, the iRNA agent is produced, and processed into sRNA agent fragments that mediate gene silencing.

Physiological Effects

The iRNA agents described herein can be designed such that determining therapeutic toxicity is made easier by the complementarity of the iRNA agent with both a human and a non-human animal sequence. By these methods, an iRNA agent can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the iRNA agent could be complementary to sequences within homologous genes, e.g., oncogenes or tumor suppressor genes, of the non-human mammal and the human. By determining the toxicity of the iRNA agent in the non-human mammal, one can extrapolate the toxicity of the iRNA agent in a human. For a more strenuous toxicity test, the iRNA agent can be complementary to a human and more than one, e.g., two or three or more, non-human animals.

The methods described herein can be used to correlate any physiological effect of an iRNA agent on a human, e.g., any unwanted effect, such as a toxic effect, or any positive, or desired effect.

Delivery Module

An RNA, e.g., an iRNA agent described herein, can be used with a drug delivery conjugate or module, such as those described herein. In addition, an iRNA agent described herein, e.g., an iRNA agent having a modification on the sense strand to inhibit off-target silencing, an iRNA agent having a non canonical pairing, an iRNA agent having a chemical modification described herein, e.g., a modification which enhances resistance to degradation, an iRNA agent having an architecture or structure described herein, an iRNA agent administered as described herein, or an iRNA agent formulated as described herein, combined with, associated with, and delivered by such a drug delivery conjugate or module.

The iRNA agents can be complexed to a delivery agent that features a modular complex. The complex can include a carrier agent linked to one or more of (preferably two or more, more preferably all three of): (a) a condensing agent (e.g., an agent capable of attracting, e.g., binding, a nucleic acid, e.g., through ionic or electrostatic interactions); (b) a fusogenic agent (e.g., an agent capable of fusing and/or being transported through a cell membrane, e.g., an endosome membrane); and (c) a targeting group, e.g., a cell or tissue targeting agent, e.g., a lectin, glycoprotein, lipid or protein, e.g., an antibody, that binds to a specified cell type such as a neural cell in the brain.

An iRNA agent, e.g., iRNA agent or sRNA agent described herein, can be linked, e.g., coupled or bound, to the modular complex. The iRNA agent can interact with the condensing agent of the complex, and the complex can be used to deliver an iRNA agent to a cell, e.g., in vitro or in vivo. For example, the complex can be used to deliver an iRNA agent to a subject in need thereof, e.g., to deliver an iRNA agent to a subject having a disorder, e.g., a disorder described herein, such as a neurodegenerative disease or disorder.

The fusogenic agent and the condensing agent can be different agents or the one and the same agent. For example, a polyamino chain, e.g., polyethyleneimine (PEI), can be the fusogenic and/or the condensing agent.

The delivery agent can be a modular complex. For example, the complex can include a carrier agent linked to one or more of (preferably two or more, more preferably all three of):
(a) a condensing agent (e.g., an agent capable of attracting, e.g., binding, a nucleic acid, e.g., through ionic interaction),
(b) a fusogenic agent (e.g., an agent capable of fusing and/or being transported through a cell membrane, e.g., an endosome membrane), and
(c) a targeting group, e.g., a cell or tissue targeting agent, e.g., a lectin, glycoprotein, lipid or protein, e.g., an antibody, that binds to a specified cell type such as a neural cell (e.g., a neural cell in the brain). A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyamino acids, multivalent galactose, transferrin, bisphosphonate, polyglutamate, polyaspartate, a lipid, cholesterol, a steroid, bile acid, folate, vitamin B12, biotin, Neproxin, or an RGD peptide or RGD peptide mimetic.

Carrier agents. The carrier agent of a modular complex described herein can be a substrate for attachment of one or more of: a condensing agent, a fusogenic agent, and a targeting group. The carrier agent would preferably lack an endogenous enzymatic activity. The agent would preferably be a biological molecule, preferably a macromolecule. Polymeric biological carriers are preferred. It would also be preferred that the carrier molecule be biodegradable.

The carrier agent can be a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or lipid. The carrier molecule can also be a recombinant or synthetic molecule, such as a synthetic polymer, e.g., a synthetic polyamino acid. Examples of polyamino acids include polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Other useful carrier molecules can be identified by routine methods.

A carrier agent can be characterized by one or more of: (a) is at least 1 Da in size; (b) has at least 5 charged groups, preferably between 5 and 5000 charged groups; (c) is present in the complex at a ratio of at least 1:1 carrier agent to fusogenic agent; (d) is present in the complex at a ratio of at least 1:1 carrier agent to condensing agent; (e) is present in the complex at a ratio of at least 1:1 carrier agent to targeting agent.

Fusogenic agents. A fusogenic agent of a modular complex described herein can be an agent that is responsive to, e.g., changes charge depending on, the pH environment. Upon encountering the pH of an endosome, it can cause a physical change, e.g., a change in osmotic properties which disrupts or increases the permeability of the endosome membrane. Preferably, the fusogenic agent changes charge, e.g., becomes protonated, at pH lower than physiological range. For example, the fusogenic agent can become protonated at pH 4.5-6.5. The fusogenic agent can serve to release the iRNA agent into the cytoplasm of a cell after the complex is taken up, e.g., via endocytosis, by the cell, thereby increasing the cellular concentration of the iRNA agent in the cell.

In one embodiment, the fusogenic agent can have a moiety, e.g., an amino group, which, when exposed to a specified pH range, will undergo a change, e.g., in charge, e.g., protonation. The change in charge of the fusogenic agent can trigger a change, e.g., an osmotic change, in a vesicle, e.g., an endocytic vesicle, e.g., an endosome. For example, the fusogenic agent, upon being exposed to the pH environment of an endosome, will cause a solubility or osmotic change substantial enough to increase the porosity of (preferably, to rupture) the endosomal membrane.

The fusogenic agent can be a polymer, preferably a polyamino chain, e.g., polyethyleneimine (PEI). The PEI can be linear, branched, synthetic or natural. The PEI can be, e.g., alkyl substituted PEI, or lipid substituted PEI.

In other embodiments, the fusogenic agent can be polyhistidine, polyimidazole, polypyridine, polypropyleneimine, mellitin, or a polyacetal substance, e.g., a cationic polyacetal. In some embodiment, the fusogenic agent can have an alpha helical structure. The fusogenic agent can be a membrane disruptive agent, e.g., mellittin.

A fusogenic agent can have one or more of the following characteristics: (a) is at least 1 Da in size; (b) has at least 10 charged groups, preferably between 10 and 5000 charged groups, more preferably between 50 and 1000 charged groups; (c) is present in the complex at a ratio of at least 1:1 fusogenic agent to carrier agent; (d) is present in the complex at a ratio of at least 1:1 fusogenic agent to condensing agent; (e) is present in the complex at a ratio of at least 1:1 fusogenic agent to targeting agent.

Other suitable fusogenic agents can be tested and identified by a skilled artisan. The ability of a compound to respond to, e.g., change charge depending on, the pH environment can be tested by routine methods, e.g., in a cellular assay. For example, a test compound is combined or contacted with a cell, and the cell is allowed to take up the test compound, e.g., by endocytosis. An endosome preparation can then be made from the contacted cells and the endosome preparation compared to an endosome preparation from control cells. A change, e.g., a decrease, in the endosome fraction from the contacted cell vs. the control cell indicates that the test compound can function as a fusogenic agent. Alternatively, the contacted cell and control cell can be evaluated, e.g., by microscopy, e.g., by light or electron microscopy, to determine a difference in endosome population in the cells. The test compound can be labeled. In another type of assay, a modular complex described herein is constructed using one or more test or putative fusogenic agents. The modular complex can be constructed using a labeled nucleic acid instead of the iRNA. A two-step assay can be performed, wherein a first assay evaluates the ability of a test compound alone to respond to, e.g., change charge depending on, the pH environment; and a second assay evaluates the ability of a modular complex that includes the test compound to respond to, e.g., change charge depending on, the pH environment.

Condensing agent. The condensing agent of a modular complex described herein can interact with (e.g., attracts, holds, or binds to) an iRNA agent and act to (a) condense, e.g., reduce the size or charge of the iRNA agent and/or (b) protect the iRNA agent, e.g., protect the iRNA agent against degradation. The condensing agent can include a moiety, e.g., a charged moiety, that can interact with a nucleic acid, e.g., an iRNA agent, e.g., by ionic interactions. The condensing agent would preferably be a charged polymer, e.g., a polycationic chain. The condensing agent can be a polylysine (PLL), spermine, spermidine, polyamine, pseudopeptide-polyamine, peptidomimetic polyamine, dendrimer polyamine, arginine, amidine, protamine, cationic lipid, cationic porphyrin, quarternary salt of a polyamine, or an alpha helical peptide.

A condensing agent can have the following characteristics: (a) at least 1 Da in size; (b) has at least 2 charged groups, preferably between 2 and 100 charged groups; (c) is present in the complex at a ratio of at least 1:1 condensing agent to carrier agent; (d) is present in the complex at a ratio of at least 1:1 condensing agent to fusogenic agent; (e) is present in the complex at a ratio of at least 1:1 condensing agent to targeting agent.

Other suitable condensing agents can be tested and identified by a skilled artisan, e.g., by evaluating the ability of a test agent to interact with a nucleic acid, e.g., an iRNA agent. The ability of a test agent to interact with a nucleic acid, e.g., an iRNA agent, e.g., to condense or protect the iRNA agent, can be evaluated by routine techniques. In one assay, a test agent is contacted with a nucleic acid, and the size and/or charge of the contacted nucleic acid is evaluated by a technique suitable to detect changes in molecular mass and/or charge. Such techniques include non-denaturing gel electrophoresis, immunological methods, e.g., immunoprecipitation, gel filtration, ionic interaction chromatography, and the like. A test agent is identified as a condensing agent if it changes the mass and/or charge (preferably both) of the contacted nucleic acid, compared to a control. A two-step assay can also be performed, wherein a first assay evaluates the ability of a test compound alone to interact with, e.g., bind to, e.g., condense the charge and/or mass of, a nucleic cid; and a second assay evaluates the ability of a modular complex that includes the test compound to interact with, e.g., bind to, e.g., condense the charge and/or mass of, a nucleic acid.

Amphipathic Delivery Agents

An RNA, e.g., an iRNA agent, described herein can be used with an amphipathic delivery conjugate or module, such as those described herein. In addition, an iRNA agent described herein, e.g., an iRNA agent having a modification on the sense strand to inhibit off-target silencing, an iRNA agent having a noncanonical pairing, an iRNA agent having a chemical modification described herein, e.g., a modification which enhances resistance to degradation, an iRNA agent having an architecture or structure described herein, an iRNA agent administered as described herein, or an iRNA agent formulated as described herein, combined with, associated with, and delivered by such an amphipathic delivery conjugate.

An amphipathic molecule is a molecule having a hydrophobic and a hydrophilic region. Such molecules can interact with (e.g., penetrate or disrupt) lipids, e.g., a lipid bilayer of a cell. As such, they can serve as delivery agent for an associated (e.g., bound) iRNA (e.g., an iRNA or sRNA described herein). A preferred amphipathic molecule to be used in the compositions described herein (e.g., the amphipathic iRNA constructs described herein) is a polymer. The polymer may have a secondary structure, e.g., a repeating secondary structure.

One example of an amphipathic polymer is an amphipathic polypeptide, e.g., a polypeptide having a secondary structure such that the polypeptide has a hydrophilic and a hybrophobic face. The design of amphipathic peptide structures (e.g., alpha-helical polypeptides) is routine to one of skill in the art. For example, the following references provide guidance: Grell et al. (2001) J Pept Sci 7(3):146-51; Chen et al. (2002) J Pept Res 59(1):18-33; Iwata et al. (1994) J Biol Chem 269(7):4928-33; Comut et al. (1994) FEBS Lett 349(1):29-33; Negrete et al. (1998) Protein Sci 7(6):1368-79.

Another example of an amphipathic polymer is a polymer made up of two or more amphipathic subunits, e.g., two or more subunits containing cyclic moieties (e.g., a cyclic moiety having one or more hydrophilic groups and one or more hydrophobic groups). For example, the subunit may contain a steroid, e.g., cholic acid; or a aromatic moiety. Such moieties preferably can exhibit atropisomerism, such that they can form opposing hydrophobic and hydrophilic faces when in a polymer structure.

The ability of a putative amphipathic molecule to interact with a lipid membrane, e.g., a cell membrane, can be tested by routine methods, e.g., in a cell free or cellular assay. For example, a test compound is combined or contacted with a synthetic lipid bilayer, a cellular membrane fraction, or a cell, and the test compound is evaluated for its ability to interact with, penetrate, or disrupt the lipid bilayer, cell membrane or cell. The test compound can be labeled in order to detect the interaction with the lipid bilayer, cell membrane, or cell. In another type of assay, the test compound is linked to a reporter molecule or an iRNA agent (e.g., an iRNA or sRNA described herein), and the ability of the reporter molecule or iRNA agent to penetrate the lipid bilayer, cell membrane or cell is evaluated. A two-step assay can also be performed, wherein a first assay evaluates the ability of a test compound alone to interact with a lipid bilayer, cell membrane or cell; and a second assay evaluates the ability of a construct (e.g., a construct described herein) that includes the test compound and a reporter or iRNA agent to interact with a lipid bilayer, cell membrane or cell.

An amphipathic polymer useful in the compositions described herein has at least 2, preferably at least 5, more preferably at least 10, 25, 50, 100, 200, 500, 1000, 2000, 50000 or more subunits (e.g., amino acids or cyclic subunits). A single amphipathic polymer can be linked to one or more, e.g., 2, 3, 5, 10 or more iRNA agents (e.g., iRNA or sRNA agents described herein). In some embodiments, an amphipathic polymer can contain both amino acid and cyclic subunits, e.g., aromatic subunits.

The invention features a composition that includes an iRNA agent (e.g., an iRNA or sRNA described herein) in association with an amphipathic molecule. Such compositions may be referred to herein as “amphipathic iRNA constructs.” Such compositions and constructs are useful in the delivery or targeting of iRNA agents, e.g., delivery or targeting of iRNA agents to a cell. While not wanting to be bound by theory, such compositions and constructs can increase the porosity of, e.g., can penetrate or disrupt, a lipid (e.g., a lipid bilayer of a cell), e.g., to allow entry of the iRNA agent into a cell.

In one aspect, the invention relates to a composition comprising an iRNA agent (e.g., an iRNA or sRNA agent described herein) linked to an amphipathic molecule. The iRNA agent and the amphipathic molecule may be held in continuous contact with one another by either covalent or noncovalent linkages.

The amphipathic molecule of the composition or construct is preferably other than a phospholipid, e.g., other than a micelle, membrane or membrane fragment.

The amphipathic molecule of the composition or construct is preferably a polymer. The polymer may include two or more amphipathic subunits. One or more hydrophilic groups and one or more hydrophobic groups may be present on the polymer. The polymer may have a repeating secondary structure as well as a first face and a second face. The distribution of the hydrophilic groups and the hydrophobic groups along the repeating secondary structure can be such that one face of the polymer is a hydrophilic face and the other face of the polymer is a hydrophobic face.

The amphipathic molecule can be a polypeptide, e.g., a polypeptide comprising an α-helical conformation as its secondary structure.

In one embodiment, the amphipathic polymer includes one or more subunits containing one or more cyclic moiety (e.g., a cyclic moiety having one or more hydrophilic groups and/or one or more hydrophobic groups). In one embodiment, the polymer is a polymer of cyclic moieties such that the moieties have alternating hydrophobic and hydrophilic groups. For example, the subunit may contain a steroid, e.g., cholic acid. In another example, the subunit may contain an aromatic moiety. The aromatic moiety may be one that can exhibit atropisomerism, e.g., a 2,2′-biS(substituted)-1-1′-binaphthyl or a 2,2′-biS(substituted) biphenyl. A subunit may include an aromatic moiety of Formula (M):

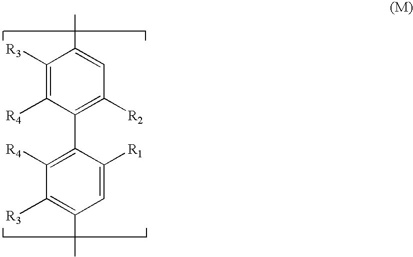

The invention features a composition that includes an iRNA agent (e.g., an iRNA or sRNA described herein) in association with an amphipathic molecule. Such compositions may be referred to herein as “amphipathic iRNA constructs.” Such compositions and constructs are useful in the delivery or targeting of iRNA agents, e.g., delivery or targeting of iRNA agents to a cell. While not wanting to be bound by theory, such compositions and constructs can increase the porosity of, e.g., can penetrate or disrupt, a lipid (e.g., a lipid bilayer of a cell), e.g., to allow entry of the iRNA agent into a cell.

Referring to Formula M, R₁ is C₁-C₁₀₀ alkyl optionally substituted with aryl, alkenyl, alkynyl, alkoxy or halo and/or optionally inserted with O, S, alkenyl or alkynyl; C₁-C₁₀₀ perfluoroalkyl; or OR₅.

R₂ is hydroxy; nitro; sulfate; phosphate; phosphate ester; sulfonic acid; OR₆; or C₁-C₁₀₀ alkyl optionally substituted with hydroxy, halo, nitro, aryl or alkyl sulfinyl, aryl or alkyl sulfonyl, sulfate, sulfonic acid, phosphate, phosphate ester, substituted or unsubstituted aryl, carboxyl, carboxylate, amino carbonyl, or alkoxycarbonyl, and/or optionally inserted with O, NH, S, S(O), SO₂, alkenyl, or alkynyl.

R₃ is hydrogen, or when taken together with R₄ forms a fused phenyl ring.

R₄ is hydrogen, or when taken together with R₃ forms a fused phenyl ring.

R₅ is C₁-C₁₀₀ alkyl optionally substituted with aryl, alkenyl, alkynyl, alkoxy or halo and/or optionally inserted with O, S, alkenyl or alkynyl; or C₁-C₁₀₀ perfluoroalkyl; and R₆ is C₁-C₁₀₀ alkyl optionally substituted with hydroxy, halo, nitro, aryl or alkyl sulfinyl, aryl or alkyl sulfonyl, sulfate, sulfonic acid, phosphate, phosphate ester, substituted or unsubstituted aryl, carboxyl, carboxylate, amino carbonyl, or alkoxycarbonyl, and/or optionally inserted with O, NH, S, S(O), SO₂, alkenyl, or alkynyl.

Increasing Cellular Uptake of dsRNAs

A method of the invention that can include the administration of an iRNA agent and a drug that affects the uptake of the iRNA agent into the cell. The drug can be administered before, after, or at the same time that the iRNA agent is administered. The drug can be covalently linked to the iRNA agent. The drug can have a transient effect on the cell.

The drug can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

iRNA Conjugates

An iRNA agent can be coupled, e.g., covalently coupled, to a second agent. For example, an iRNA agent used to treat a particular disorder can be coupled to a second therapeutic agent, e.g., an agent other than the iRNA agent. The second therapeutic agent can be one which is directed to the treatment of the same disorder.

iRNA Production

An iRNA can be produced, e.g., in bulk, by a variety of methods. Exemplary methods include: organic synthesis and RNA cleavage, e.g., in vitro cleavage.

Organic Synthesis. An iRNA can be made by separately synthesizing each respective strand of a double-stranded RNA molecule. The component strands can then be annealed.

A large bioreactor, e.g., the OligoPilot II from Pharmacia Biotec AB (Uppsala Sweden), can be used to produce a large amount of a particular RNA strand for a given iRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the iRNA. Typically, the two complementary strands are produced separately and then annealed, e.g., after release from the solid support and deprotection.

Organic synthesis can be used to produce a discrete iRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsRNA Cleavage. iRNAs can also be made by cleaving a larger ds iRNA. The cleavage can be mediated in vitro or in vivo. For example, to produce iRNAs by cleavage in vitro, the following method can be used:

In vitro transcription. dsRNA is produced by transcribing a nucleic acid (DNA) segment in both directions. For example, the HiScribe™ RNAi transcription kit (New England Biolabs) provides a vector and a method for producing a dsRNA for a nucleic acid segment that is cloned into the vector at a position flanked on either side by a T7 promoter. Separate templates are generated for T7 transcription of the two complementary strands for the dsRNA. The templates are transcribed in vitro by addition of T7 RNA polymerase and dsRNA is produced. Similar methods using PCR and/or other RNA polymerases (e.g., T3 or SP6 polymerase) can also be used. In one embodiment, RNA generated by this method is carefully purified to remove endotoxins that may contaminate preparations of the recombinant enzymes.

In vitro cleavage. dsRNA is cleaved in vitro into iRNAs, for example, using a Dicer or comparable RNAse III-based activity. For example, the dsRNA can be incubated in an in vitro extract from Drosophila or using purified components, e.g. a purified RNAse or RISC complex (RNA-induced silencing complex). See, e.g., Ketting et al. Genes Dev 2001 Oct. 15; 15(20):2654-9. and Hammond Science 2001 Aug. 10; 293(5532): 1146-50.

dsRNA cleavage generally produces a plurality of iRNA species, each being a particular 21 to 23 nt fragment of a source dsRNA molecule. For example, iRNAs that include sequences complementary to overlapping regions and adjacent regions of a source dsRNA molecule may be present.

Regardless of the method of synthesis, the iRNA preparation can be prepared in a solution (e.g., an aqueous and/or organic solution) that is appropriate for formulation. For example, the iRNA preparation can be precipitated and redissolved in pure double-distilled water, and lyophilized. The dried iRNA can then be resuspended in a solution appropriate for the intended formulation process.

Synthesis of modified and nucleotide surrogate iRNA agents is discussed below.

Formulation

The iRNA agents described herein can be formulated for administration to a subject.

For ease of exposition, the formulations, compositions, and methods in this section are discussed largely with regard to unmodified iRNA agents. It should be understood, however, that these formulations, compositions, and methods can be practiced with other iRNA agents, e.g., modified iRNA agents, and such practice is within the invention.

A formulated iRNA composition can assume a variety of states. In some examples, the composition is at least partially crystalline, uniformly crystalline, and/or anhydrous (e.g., less than 80, 50, 30, 20, or 10% water). In another example, the iRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the iRNA composition is formulated in a manner that is compatible with the intended method of administration.

In particular embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

A iRNA preparation can be formulated in combination with another agent, e.g., another therapeutic agent or an agent that stabilizes a iRNA, e.g., a protein that complexes with iRNA to form an iRNP. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the iRNA preparation includes another iRNA agent, e.g., a second iRNA that can mediated RNAi with respect to a second gene, or with respect to the same gene. Still other preparation can include at least three, five, ten, twenty, fifty, or a hundred or more different iRNA species. Such iRNAs can mediated RNAi with respect to a similar number of different genes.

In one embodiment, the iRNA preparation includes at least a second therapeutic agent (e.g., an agent other than an RNA or a DNA).

Targeting

For ease of exposition the formulations, compositions and methods in this section are discussed largely with regard to unmodified iRNAs. It should be understood, however, that these formulations, compositions and methods can be practiced with other iRNA agents, e.g., modified iRNA agents, and such practice is within the invention.

In some embodiments, an iRNA agent, e.g., a double-stranded iRNA agent, or sRNA agent, (e.g., a precursor, e.g., a larger iRNA agent which can be processed into a sRNA agent, or a DNA which encodes an iRNA agent, e.g., a double-stranded iRNA agent, or sRNA agent, or precursor thereof) is targeted to a particular cell. For example, a liposome or particle or other structure that includes a iRNA can also include a targeting moiety that recognizes a specific molecule on a target cell. The targeting moiety can be a molecule with a specific affinity for a target cell. Targeting moieties can include antibodies directed against a protein found on the surface of a target cell, or the ligand or a receptor-binding portion of a ligand for a molecule found on the surface of a target cell.

An antigen, can be used to target an iRNA to a neural cell in the brain.

In one embodiment, the targeting moiety is attached to a liposome. For example, U.S. Pat. No. 6,245,427 describes a method for targeting a liposome using a protein or peptide. In another example, a cationic lipid component of the liposome is derivatized with a targeting moiety. For example, WO 96/37194 describes converting N-glutaryldioleoylphosphatidyl ethanolamine to a N-hydroxysuccinimide activated ester. The product was then coupled to an RGD peptide.

Treatment Methods and Routes of Delivery

The pharmaceutical compositions of the present invention may be administered in a number of ways depending upon whether local or systemic treatment is desired and upon the area to be treated. Administration may be topical (including ophthalmic, intranasal, transdermal), oral or parenteral. Parenteral administration includes intravenous drip, subcutaneous, intraperitoneal or intramuscular injection, or intrathecal or intraventricular administration. The route of delivery can be dependent on the disorder of the patient.

An iRNA agent can be modified such that it is capable of traversing the blood brain barrier. For example, the iRNA agent can be conjugated to a molecule that enables the agent to traverse the barrier. Such modified iRNA agents can be administered by any desired method, such as by intraventricular or intramuscular injection, or by pulmonary delivery, for example.

An iRNA agent can be administered ocularly, such as to treat retinal disorder, e.g., a retinopathy. For example, the pharmaceutical compositions can be applied to the surface of the eye or nearby tissue, e.g., the inside of the eyelid. They can be applied topically, e.g., by spraying, in drops, as an eyewash, or an ointment. Ointments or droppable liquids may be delivered by ocular delivery systems known in the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. The pharmaceutical composition can also be administered to the interior of the eye, and can be introduced by a needle or other delivery device which can introduce it to a selected area or structure. The composition containing the iRNA agent can also be applied via an ocular patch.

Administration can be provided by the subject or by another person, e.g., a another caregiver. A caregiver can be any entity involved with providing care to the human: for example, a hospital, hospice, doctor's office, outpatient clinic; a healthcare worker such as a doctor, nurse, or other practitioner; or a spouse or guardian, such as a parent. The medication can be provided in measured doses or in a dispenser which delivers a metered dose.

The subject can be monitored for reactions to the treatment, such as edema or hemorrhaging. For example, the patient can be monitored by MRI, such as daily or weekly following injection, and at periodic time intervals following injection.

The subject can also be monitored for an improvement or stabilization of disease symptoms.

In general, an iRNA agent can be administered by any suitable method. As used herein, topical delivery can refer to the direct application of an iRNA agent to any surface of the body, including the eye, a mucous membrane, surfaces of a body cavity, or to any internal surface. Formulations for topical administration may include transdermal patches, ointments, lotions, creams, gels, drops, sprays, and liquids. Conventional pharmaceutical carriers, aqueous, powder or oily bases, thickeners and the like may be necessary or desirable. Topical administration can also be used as a means to selectively deliver the iRNA agent to the epidermis or dermis of a subject, or to specific strata thereof, or to an underlying tissue.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

An iRNA agent can be administered to a subject by pulmonary delivery. Pulmonary delivery compositions can be delivered by inhalation by the patient of a dispersion so that the composition, preferably iRNA, within the dispersion can reach the lung where it can be readily absorbed through the alveolar region directly into blood circulation. Pulmonary delivery can be effective both for systemic delivery and for localized delivery to treat diseases of the lungs. In one embodiment, an anti-SNCA iRNA agent administered by pulmonary delivery has been modified such that it is capable of traversing the blood brain barrier.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self contained. Dry powder dispersion devices, for example, deliver drugs that may be readily formulated as dry powders. An iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A preferred group of carbohydrates includes lactose, threhalose, raffinose maltodextrins, and mannitol. Suitable polypeptides include aspartame. Amino acids include alanine and glycine, with glycine being preferred.

Suitable pH adjusters or buffers include organic salts prepared from organic acids and bases, such as sodium citrate, sodium ascorbate, and the like; sodium citrate is preferred.

An iRNA agent can be administered by an oral and nasal delivery. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily. In one embodiment, an iRNA agent administered by oral or nasal delivery has been modified to be capable of traversing the blood-brain barrier.

In one embodiment, unit doses or measured doses of a composition that include iRNA are dispensed by an implanted device. The device can include a sensor that monitors a parameter within a subject. For example, the device can include a pump, such as an osmotic pump and, optionally, associated electronics.

An iRNA agent can be packaged in a viral natural capsid or in a chemically or enzymatically produced artificial capsid or structure derived therefrom.

Dosage. An iRNA agent can be administered at a unit dose less than about 1.4 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×1016 copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA agent per kg of bodyweight. The unit dose, for example, can be administered by injection (e.g., intravenous or intramuscular, intrathecally, or directly into the brain), an inhaled dose, or a topical application. Particularly preferred dosages are less than 2, 1, or 0.1 mg/kg of body weight.

Delivery of an iRNA agent directly to an organ (e.g., directly to the brain) can be at a dosage on the order of about 0.00001 mg to about 3 mg per organ, or preferably about 0.0001-0.001 mg per organ, about 0.03-3.0 mg per organ, about 0.1-3.0 mg per eye or about 0.3-3.0 mg per organ.

In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In one embodiment, the effective dose is administered with other traditional therapeutic modalities.

In one embodiment, a subject is administered an initial dose, and one or more maintenance doses of an iRNA agent, e.g., a double-stranded iRNA agent, or sRNA agent, (e.g., a precursor, e.g., a larger iRNA agent which can be processed into an sRNA agent, or a DNA which encodes an iRNA agent, e.g., a double-stranded iRNA agent, or sRNA agent, or precursor thereof). The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In one embodiment, the iRNA agent pharmaceutical composition includes a plurality of iRNA agent species. In another embodiment, the iRNA agent species has sequences that are non-overlapping and non-adjacent to another species with respect to a naturally occurring target sequence. In another embodiment, the plurality of iRNA agent species is specific for different naturally occurring target genes. In another embodiment, the iRNA agent is allele specific.

Following successful treatment, it may be desirable to have the patient undergo maintenance therapy to prevent the recurrence of the disease state, wherein the compound of the invention is administered in maintenance doses, ranging from 0.01 μg to 100 g per kg of body weight (see U.S. Pat. No. 6,107,094).

The concentration of the iRNA agent composition is an amount sufficient to be effective in treating or preventing a disorder or to regulate a physiological condition in humans. The concentration or amount of iRNA agent administered will depend on the parameters determined for the agent and the method of administration, e.g. nasal, buccal, or pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an iRNA agent, e.g., a double-stranded iRNA agent, or sRNA agent (e.g., a precursor, e.g., a larger iRNA agent which can be processed into a sRNA agent, or a DNA which encodes an iRNA agent, e.g., a double-stranded iRNA agent, or sRNA agent, or precursor thereof) can include a single treatment or, preferably, can include a series of treatments. It will also be appreciated that the effective dosage of an iRNA agent such as an sRNA agent used for treatment may increase or decrease over the course of a particular treatment. Changes in dosage may result and become apparent from the results of diagnostic assays as described herein. For example, the subject can be monitored after administering an iRNA agent composition. Based on information from the monitoring, an additional amount of the iRNA agent composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human gene, e.g., a gene that produces a target RNA. The transgenic animal can be deficient for the corresponding endogenous RNA. In another embodiment, the composition for testing includes an iRNA agent that is complementary, at least in an internal region, to a sequence that is conserved between the target RNA in the animal model and the target RNA in a human.

The invention is further illustrated by the following examples, which should not be construed as further limiting.

### EXAMPLES

### Example 1

### Cholesterol Conjugated to the 3′ Ends of dsRNA Inhibits Silencing Effects and Improves Cellular Uptake

Candidate dsRNAs were tested in an in vitro activity assay. HeLa cells stably expressing firefly luciferase (target) and Renilla luciferase (control) were plated into 96-well plates. SiRNAs targeting firefly luciferase were transfected into cells. Luciferase protein levels were measured to determine silencing of firefly luciferase expression as compared to the control Renilla luciferase expression.

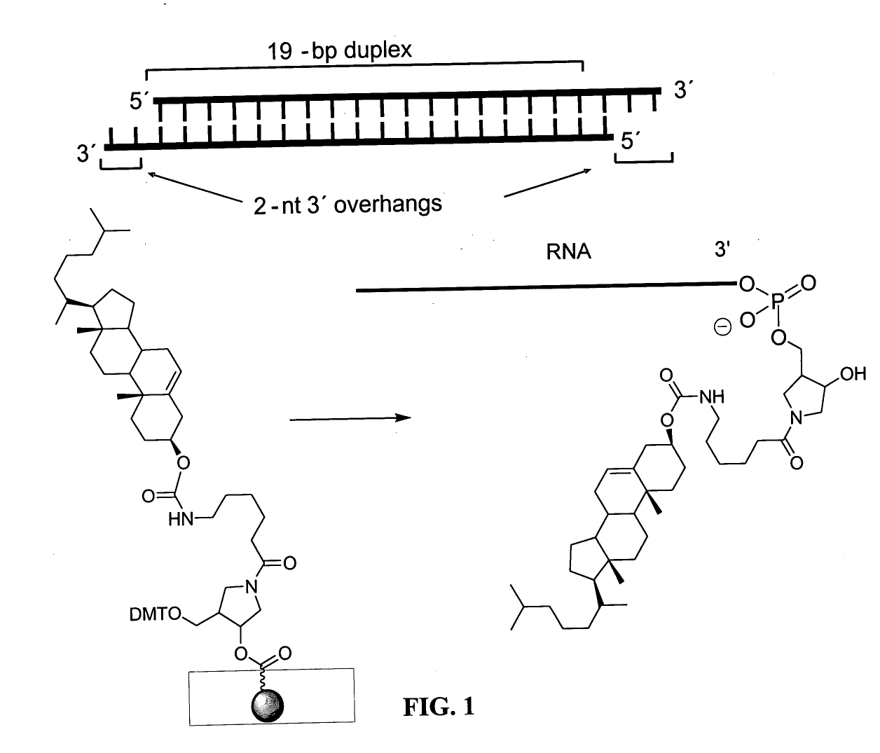

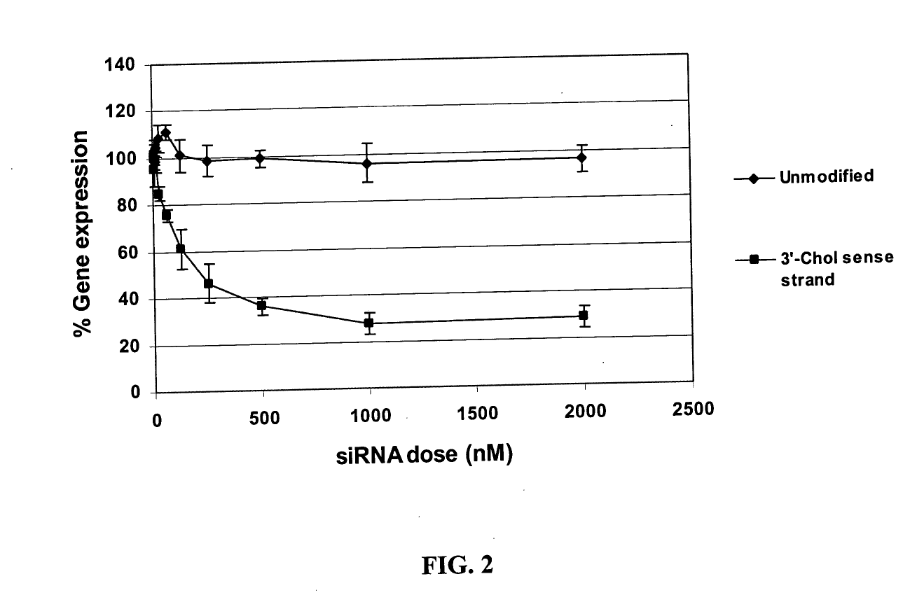

Cholesterol was conjugated to the 3′ end of a double stranded RNA (dsRNA) via a pyrrolidine linker (see FIG. 1). Unmodified dsRNA was applied to HeLa-Luc cells in the absence of transfection reagent, and no silencing of firefly luciferase was observed. However, when a dsRNA containing a cholesterol moiety conjugated to the 3′ terminus of the sense strand (“3′-Chol. Sense strand) was added to the reporter cell line (also in the absence of transfection reagent), gene expression of firefly luciferase fell to under 30% (FIG. 2). This result indicates that cholesterol conjugated to the 3′ end of the sense strand has minimal interference with the ability of the dsRNA to silence the target RNA. This result also indicates that cholesterol can increase uptake of the dsRNA into cells.

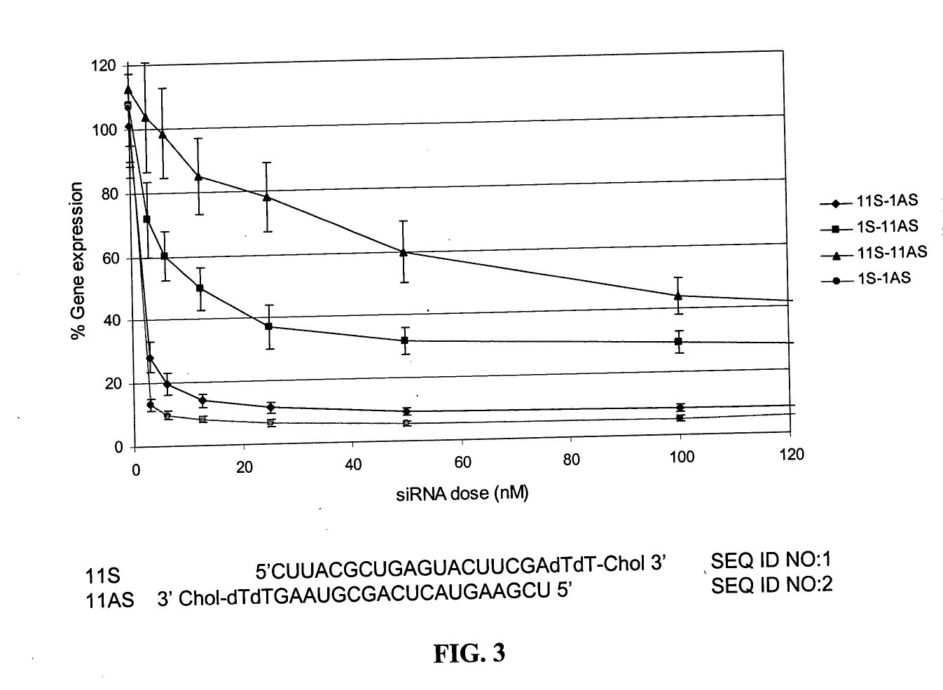

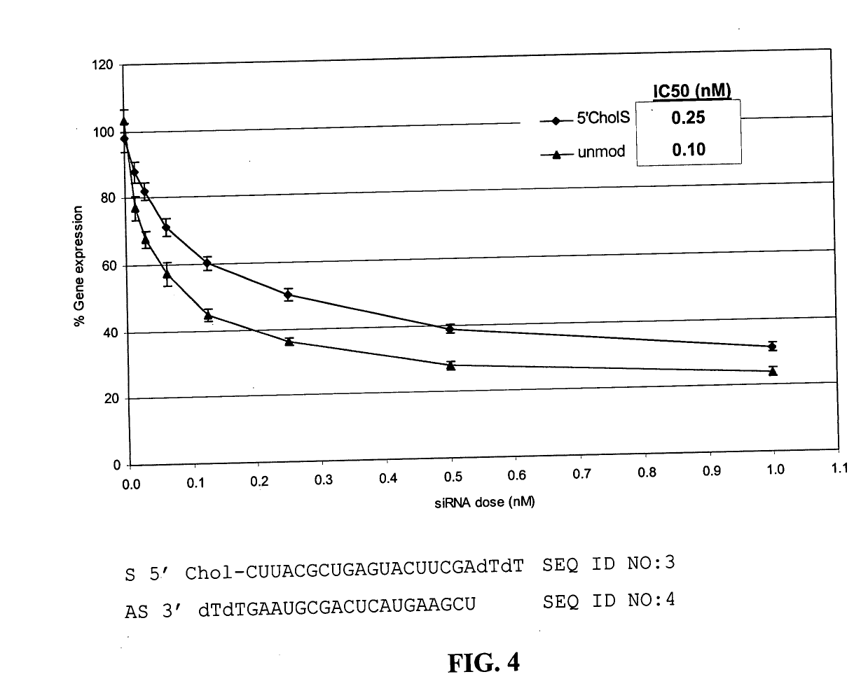

The effect of cholesterol conjugation on the sense strand and the antisense strand of a dsRNA were compared (FIG. 3). An unmodified dsRNA (called 1S-1AS (“S” indicates sense strand; “AS” indicates antisense strand; “1” indicates unmodified strand)) inhibited firefly luciferase gene expression. A dsRNA carrying a cholesterol moiety on the 3′ end of the sense strand (11S-1AS (“11” indicates cholesterol-conjugated strand)) silenced gene expression as effectively as the unmodified dsRNA. A dsRNA carrying a cholesterol moiety on the 3′ end of the antisense strand (1S-11AS) had a weaker silencing effect, and a dsRNA carrying a cholesterol moiety on the 3′ end of both strands (11S-11AS) had the weakest silencing effect. Conjugation of the cholesterol on the 5′ end of the sense strand weakened the silencing effect only slightly (FIG. 4).

Silencing of the firefly luciferase target gene was sequence dependent. A 3-sense strand cholesterol conjugated dsRNA that carried a scrambled sequence of the anti-luciferase dsRNA did not have silencing activity.

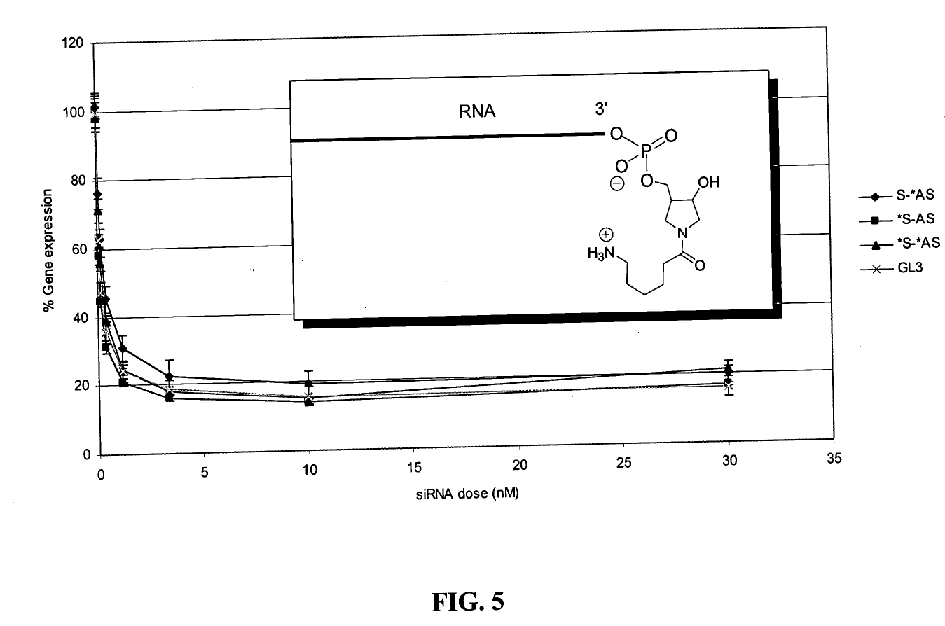

To test whether the linker between the cholesterol moiety and the RNA strand was effecting silencing, dsRNAs carrying the linker without cholesterol were tested in the HeLa cell firefly luciferase assay (FIG. 5). In this experiment, silencing was equally effective whether the dsRNA was unmodified, or if both the sense and antisense strands carried the linker, or if either the sense or the antisense strand carried the linker. Therefore, the inhibition of silencing observed for the cholesterol-conjugated dsRNAs is due to the cholesterol moiety, and not to the linker.

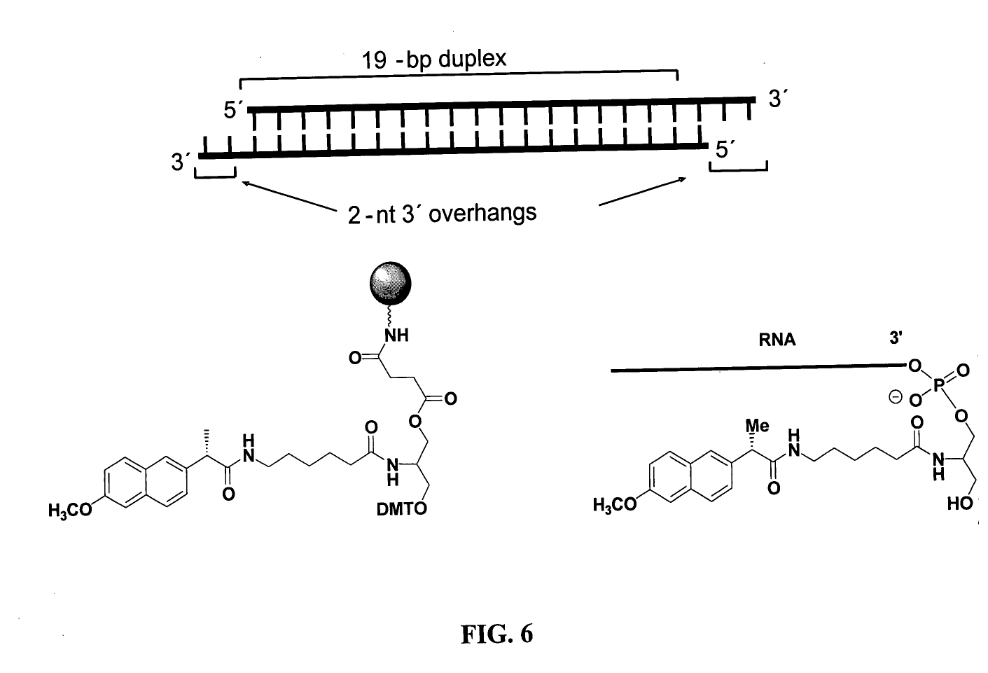

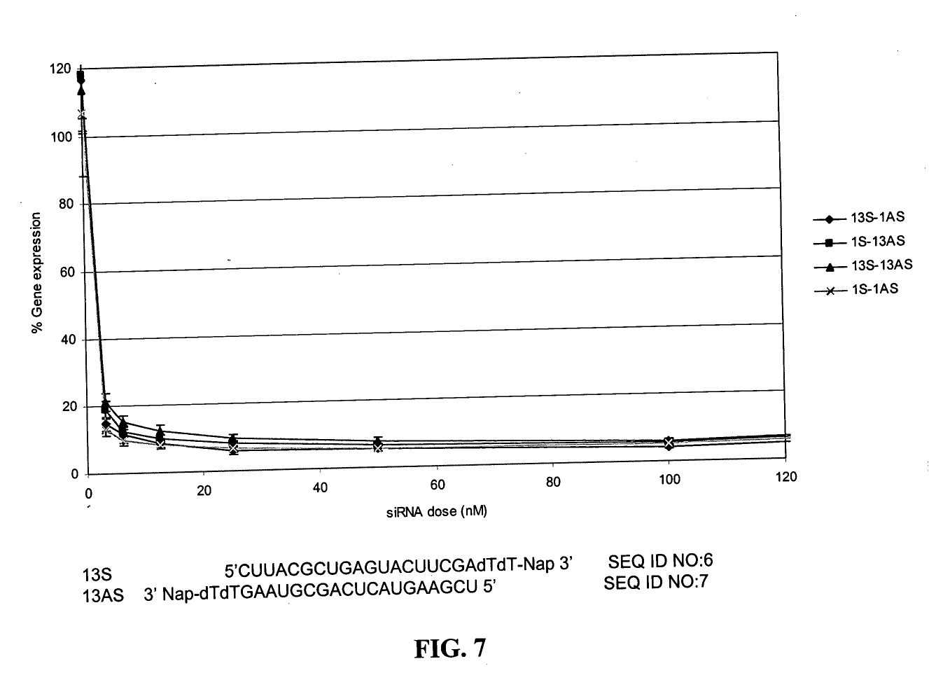

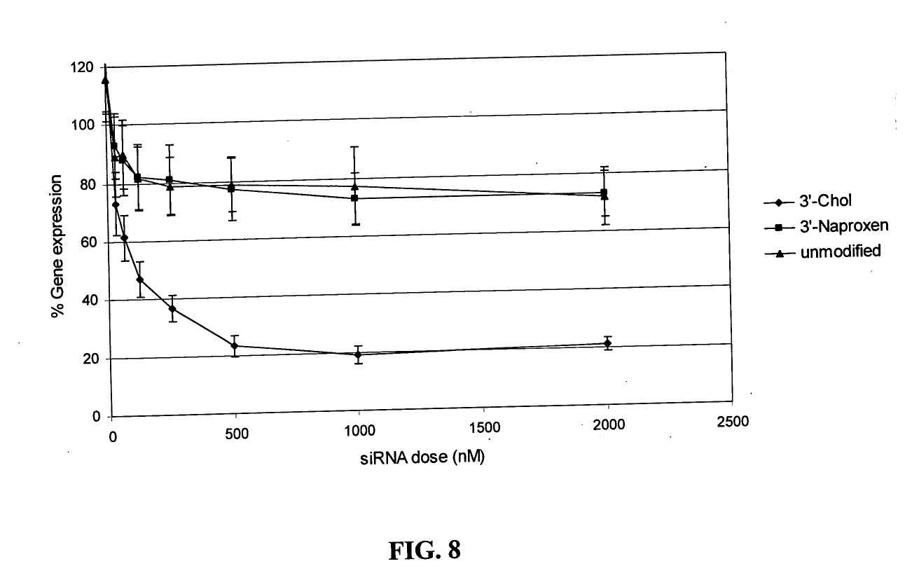

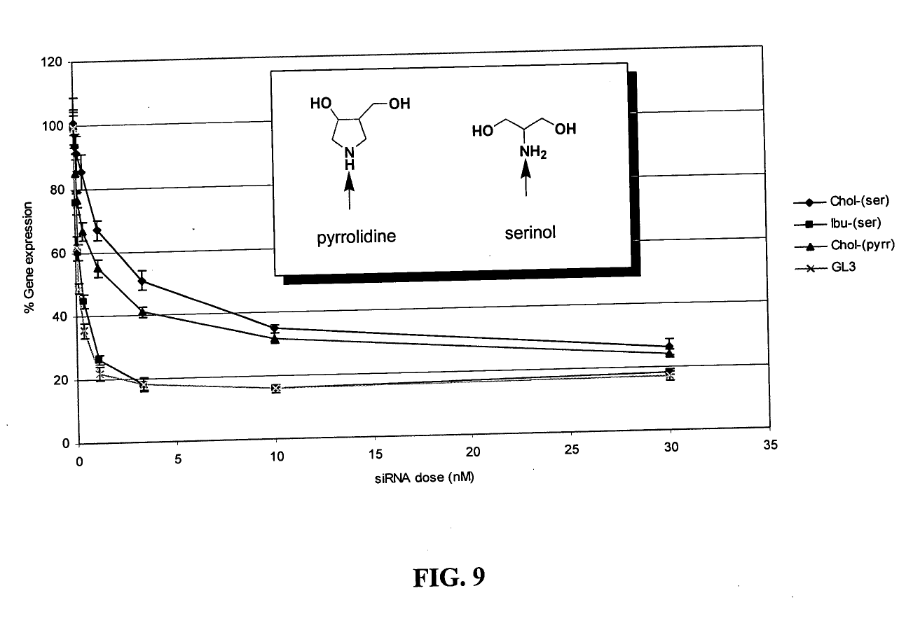

To determine whether the inhibition of silencing was specific for cholesterol, other moieties were conjugated to the 3′ ends of a dsRNA and tested for an effect on gene silencing Naproxen was tested, for example (FIG. 6). Unlike the effect of cholesterol, the dsRNAs were found to be equally effective at facilitating gene silencing in the HeLa cell firefly luciferase assay when the dsRNA was unmodified, or if both the sense and antisense strands carried naproxen, or if either the sense or the antisense strand carried naproxen (FIG. 7). Conjugation of ibuprofen also ineffective at inhibiting the silencing effect of target gene expression (see FIG. 9). Furthermore, also unlike cholesterol, 3′ conjugated naproxen (on the sense strand) did not improve cellular uptake of the dsRNA in the absence of transfection reagent (FIG. 8). Folic acid conjugate also failed to inhibit silencing when conjugated to the 3′ end of the antisense strand, and did not improve cellular uptake when tested in the absence of transfection reagent.

Different linkers can be used to conjugate a cholesterol to the 3′ end of a sense strand and cause an inhibition of gene silencing. For example, pyrrolidine and serinol linkers were each used to conjugate cholesterol to a dsRNA, and each of these molecules inhibited gene silencing to a similar extent (FIG. 9). Ibuprofen conjugated to the dsRNA via a serinol linker did not inhibit target gene expression. Transfections testing the different linkers were performed in the absence of transfection reagent.

### Example 2

### Modifications at the 5′ Ends of dsRNA Inhibit Silencing Effects

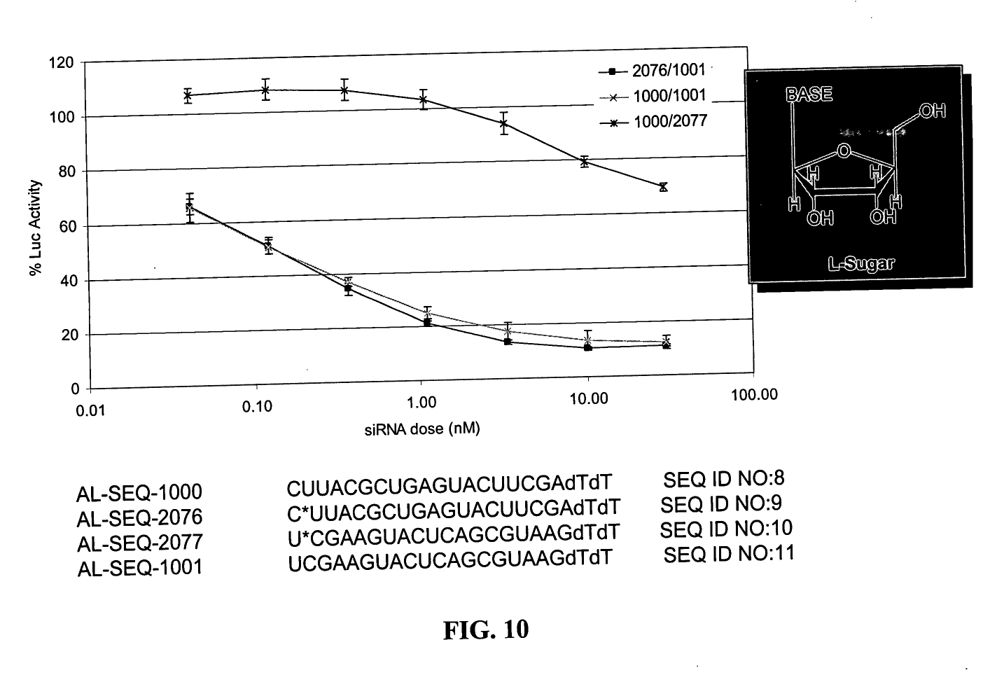

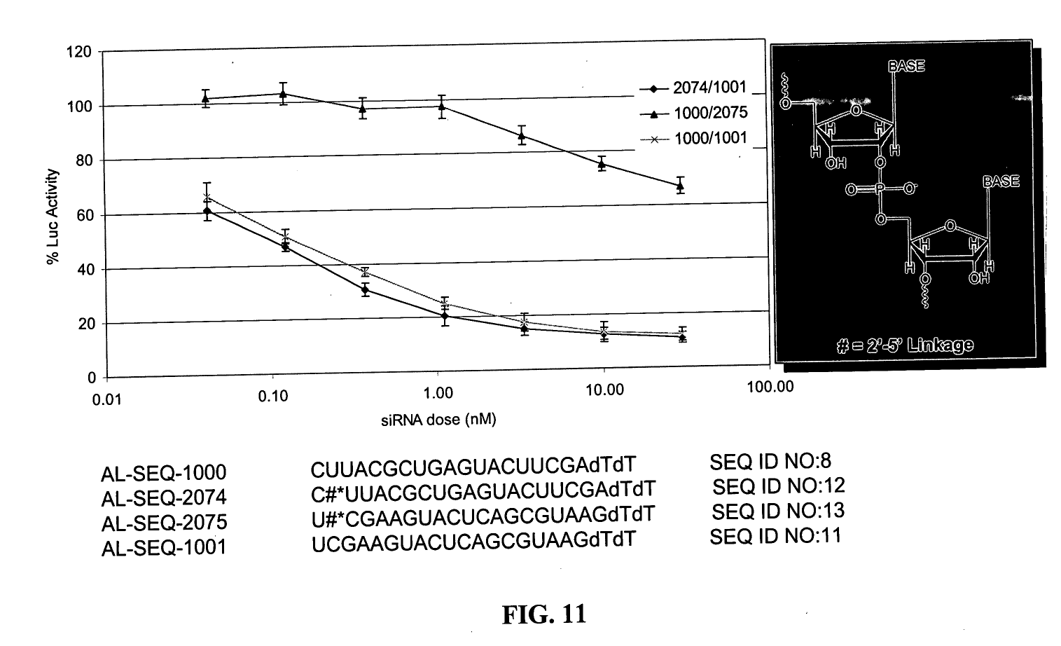

Modifications at the 5′ terminus of the antisense strand of dsRNAs inhibited silencing activity of the dsRNA. Using the HeLa cell luciferase in vitro assay, it was shown that substitution of the 5′ antisense terminal nucleotide with an L-sugar nucleotide, inhibited silencing of firefly luciferase gene expression (see 1000/2077 of FIG. 10). The same substitution on the 5′ terminus of the sense strand (2076/1001 of FIG. 10) did not affect silencing. A 2′-5′ linkage at the 5′ terminus showed a similar effect (FIG. 11).

### Example 3

### Modifications in the Internal Region of a dsRNA Inhibit Silencing Effects

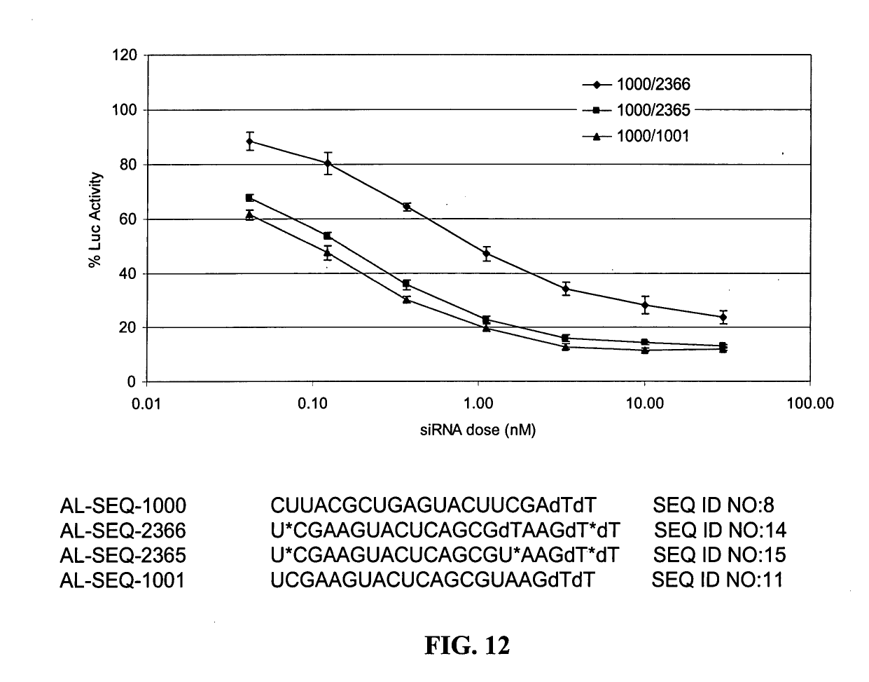

Modifications in an internal region of the antisense strand of a dsRNA inhibited silencing activity. Using the HeLa cell luciferase in vitro assay, it was shown that substitution of a uridine in the internal region of the antisense strand with a deoxythymidine weakened the silencing effect of the dsRNA (see 1000/2366 of FIG. 12). Placement of a phosphorothioate linkage group at the same uridine in the antisense strand did not affect silencing (1000/2365 of FIG. 12). DNA modifications in the sense strand of dsRNAs had zero to minimal effect on silencing. Table 11 summarizes dsRNAs having sense strand modifications and their effects on gene silencing (expressed as IC50). Modifications to antisense strands of dsRNAs and their effects on silencing (expressed as IC50) are shown in Table 12.

TABLE 11

Effect on silencing of dsRNAs
containing DNA modified sense strands.

| | | |
|---|---|---|
| AL- | | IC50 |
| SEQ- | Sequence 5′-3′^{a} | (nM) |

| | | |
|---|---|---|
| 2251 | CUU ACG CU*dG* dA*dG*dT* ACU UCG AdTdT | 0.03 |
| 2237 | CUU ACG CUG A* dG*dT* ACU UCG AdTdT | 0.04 |
| 2247 | CUU ACG CUG AGU ACU* dT*dC*dG* AdTdT | 0.04 |
| 2248 | CUU ACG CUG AGU ACU UCG* dA*dT*dT | 0.04 |
| 2250 | CUU A*dC*dG* dC*dT*G AGU ACU UCG AdTdT | 0.04 |
| 2254 | CUU ACG CUG AGU ACU UC*dG* dA*dT*dT | 0.04 |
| 2233 | CU*dT* dA*CG CUG AGU ACU UCG AdTdT | 0.07 |
| 2246 | CUU ACG CUG AGU* dA*dC*dT* UCG AdTdT | 0.07 |
| 1000 | CUU ACG CUG AGU ACU UCG AdTdT | 0.09 |
| 2232 | dC*dT*U ACG CUG AGU ACU TCG AdTdT | 0.11 |
| 2249 | dC*dT*dT* dA*CG CUG AGU ACU UCG AdTdT | 0.33 |

^{a}“d” indicates deoxynucleotide substitution; “k” indicates phosphorothioate linkage.

TABLE 12
Effect on silencing of dsRNAs
containing DNA modified antisense strands.

| | | |
|---|---|---|
| | | IC50 |
| AL-SEQ- | Sequence 5′-3′^{a} | (nM) |

| | | |
|---|---|---|
| 1000 | CUU ACG CUG AGU ACU UCG AdTdT | 0.09 |
| 2258 | dT*dC*G AAG UAC UCA GCG UAA GdTdT | 0.29 |
| 2259 | UC*dG* dA*AG UAC UCA GCG UAA GdTdT | 0.71 |
| 2264 | UCG AAG UAC UCA GCG UA*dA* dG*dTdT | 0.75 |
| 2272 | UCG AAG UAC UCA GCG UA*dA* dG*dT*dT | 0.81 |
| 2275 | UCG AAG UAC UCA GCG UA*dA* dG*dT*dT | 1.00 |
| 2273 | dT*dC*dG* dA*AG UAC UCA GCG UAA GdTdT | 1.06 |
| 2267 | UCG* dA*dA*G UAC UCA GCG* dT*AA GdTdT | 7.40 |
| 2262 | UCG AAG UAC UCA GCG dT*dA*A GdTdT | 7.81 |

^{a}“d” indicates deoxynucleotide substitution; “*” indicates phosphorothioate linkage.

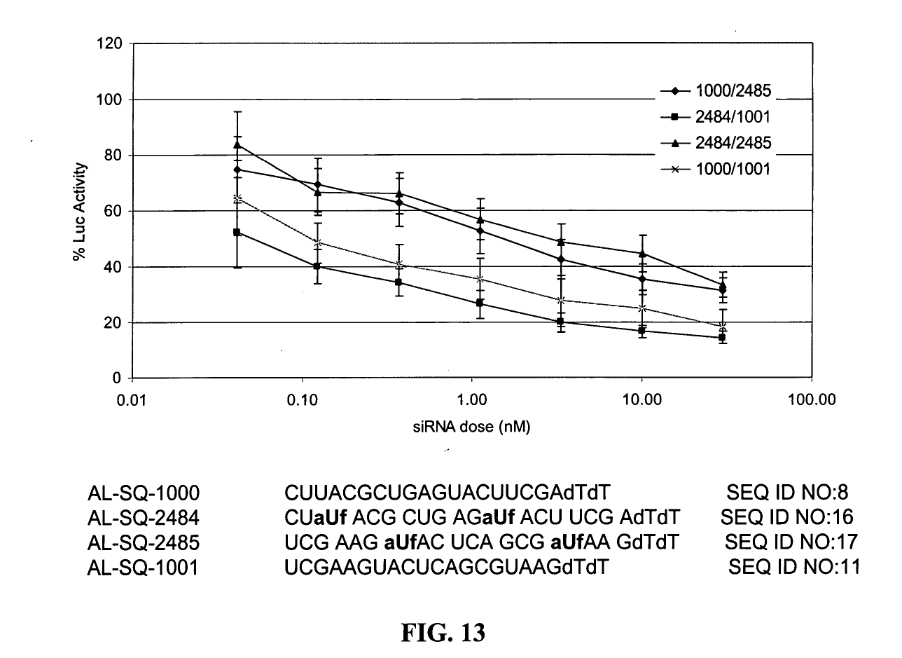

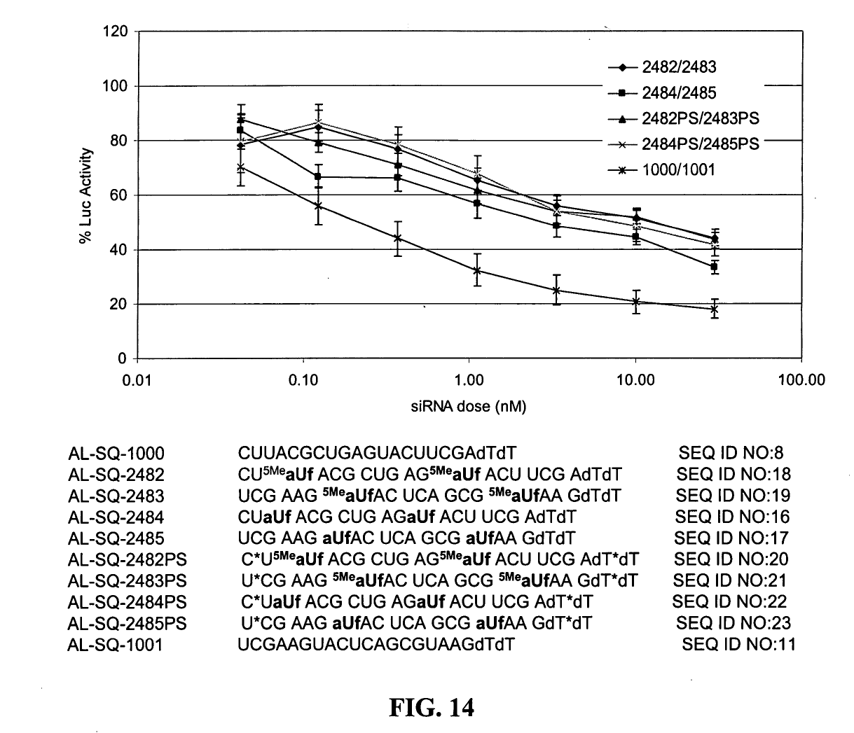

The effect of replacing a uridine in an internal region with an 2′-arabino-fluorodeoxyuridine was also tested for an effect on dsRNA silencing in the HeLa cell luciferase assay. Silencing was weakly inhibited by these modifications in the antisense strand (FIG. 13). These modifications had no effect on silencing when present only in the sense strand. A decrease in silencing was also observed when 2′-arabino-fluorodeoxyuridine was methylated or when 2′-arabino-fluorodeoxyuridine was used in combination with phosphorothioate linkages (FIG. 14).

### Other Embodiments

A number of embodiments of the invention have been described. Nevertheless, it will be understood that various modifications may be made without departing from the spirit and scope of the invention. Accordingly, other embodiments are within the scope of the following claims.

1. A method of preventing off-target gene silencing in a cell comprising contacting a duplex RNA with the cell, wherein the duplex RNA comprises a modification on the sense strand, and
(a) the sense strand of said duplex RNA has a region of at least 70% complementarity to at least 10 nucleotides of a preselected gene; or
(b) the modified or unmodified sense strand has been tested for the ability to silence the off-target gene.

2. The method of claim 1, wherein the off-target gene is expressed in said cell.

3. The method of claim 1, wherein the off-target gene is expressed in a different cell type.

4. The method of claim 1, wherein the modification is on the 5′ terminus of the sense strand.

5. The method of claim 4, wherein the modification prevents hydrolysis of a 5′ terminal phosphate group.

6. The method of claim 4, wherein the modification comprises an L-sugar at the 5′ terminus of the sense strand.

7. The method of claim 4, wherein the modification comprises an alpha-nucleotide at the 5′ terminus of the sense strand.

8. The method of claim 2, wherein the modification comprises a 2′-5′ linkage.

9. The method of claim 1, wherein the modification is on the 3′ terminus of the sense strand.

10. The method of claim 9, wherein the modification is a steroidal compound conjugated to the 3′ terminal nucleotide of the sense strand.

11. The method of claim 10, wherein the steroidal compound is cholesterol.

12. The method of claim 10, wherein the steroidal compound is conjugated to the 3′ terminal nucleotide by a cationic linker.

13. The method of claim 1, wherein the modification is on a nucleotide that is not a terminal nucleotide.

14. The method of claim 13, wherein the modification causes the sense strand to have a DNA-like conformation.

15. The method of claim 13, wherein the modification is replacement of a ribonucleotide with a deoxyribonucleotide.

16. The method of claim 13, wherein the modification is replacement of a uridine with 2′-arabino-fluorodeoxyuridine.

17. The method of claim 16, wherein the 2′-arabino-fluorodeoxyuridine is methylated.

18. A method of evaluating a modification of a sense strand of a duplex RNA for the ability to inhibit silencing of an off-target gene, the method comprising:
(a) modifying the sense strand of the duplex RNA, wherein the sense strand of said duplex RNA has a region of at least 70% complementarity for at least 10 nucleotides of the off-target gene,
(b) contacting the modified sense strand to a cell expressing the off-target gene;
(c) comparing expression of the off-target gene to expression of the off-target gene following contact with an unmodified sense strand of the duplex RNA.
